(19) United States
(12) Patent Application Publication
(10) Pub. No.: US 2006/0047171 A1
(43) Pub. Date: Mar. 2, 2006

(54) Catalytic hydrogenation of nitriles to produce capsaicinoid derivatives and amine compounds, and methods for purifying and obtaining the polymorphs thereof

(76) Inventors: Harold Meckler, Delmar, NY (US); Karl F. Popp, Schodack Landing, NY (US); Bingidimi I. Mobele, Altamont, NY (US); Paul K. Isbester, Castleton, NY (US); Bruce J. Elder, Wynantskill, NY (US); Paul F. Vogt, Arlington Heights, IL (US); Benjamin J. Littler, Mount Prospect, IL (US); Stephen A. Eastham, Rexford, NY (US); David P. Reed, Clifton Park, NY (US); Luckner G. Ulysse, Albany, NY (US); Michael D. Uttley, Delmar, NY (US)

Correspondence Address: NATH & ASSOCIATES, 112 South West Street, Alexandria, VA, 22314, US

(21) Appl. No.: 10/927,493

(22) Filed: Aug. 27, 2004

Related U.S. Application Data
(60) Provisional application No. 60/530,985, filed on Dec. 22, 2003.

(51) Int. Cl. A61K 31/16; C07C 233/24

(52) U.S. Cl. 564/219; 514/630

(57) ABSTRACT

Processes for preparing an amine compound by catalytically hydrogenating a precursor nitrile compound. In a particular aspect, the present hydrogenation process occurs in a dipolar organic solvent in the presence of a palladium/carbon catalyst and a strong anhydrous protic acid. In a further aspect, the preferred embodiment relates to a process for deprotecting a compound to produce an amine compound. In yet a further aspect, the preferred embodiment relates to amine products produced by the present processes. These amine products may be used for a variety of purposes.

The application claims priority to and the benefit of U.S. Provisional Patent Application Ser. No. 60/530,985, filed on Dec. 22, 2003, the entire contents of which are hereby incorporated by reference, under 35 U.S.C. § 119(e).

### FIELD OF THE INVENTION

The present subject matter relates to novel polymorphs of 2-[4-(2-aminoethoxy)-3-methoxyphenyl]-N-[3-(3,4-dimethylphenyl)propyl]acetamide (DA-5018) and related amines and processes for obtaining these polymorphs. In a preferred embodiment, the present subject matter also relates to novel generalized processes for the catalytic reduction of a nitrile to an amine, and more specifically to novel processes for preparing capsaicinoid derivatives, e.g. DA-5018, which have powerful anti-inflammatory and analgesic activities, as well as pharmaceutical compositions, formulations, dosage forms and methods of treatment thereof.

### BACKGROUND OF THE INVENTION

Natural capsaicin (trans-8-methyl-N-vanillyl-6-nonenamide) is a compound derived from the genus of Capsicum pepper plants and is a useful analgesic for the treatment of pain and inflammation. Synthetic capsaicin is similarly known, see e.g. LaHann, U.S. Pat. No. 4,313,958; LaHann et al., U.S. Pat. No. 4,424,205; and Gardner et al., EP Patent No. 0,282,177. However, capsaicin applied topically can irritate the skin, and, in larger doses, can cause reddening of the skin, blisters and have other toxic effects.

Chemical modification of capsaicin has yielded “burn-less” capsaicinoid, i.e. capsaicin-like, compounds that do not exhibit some of these side effects while retaining the beneficial analgesic properties of capsaicin. For example, Park et al., U.S. Pat. Nos. 5,242,944 and 5,670,546, describe certain phenylacetamide derivatives with such properties. However, the previously known synthetic process for manufacturing such compounds has numerous disadvantages.

From a synthetic standpoint, previous production of phenylacetamide derivatives has been complex and expensive, even requiring as many as 13 steps or more, as described in the '944 and '546 patents to Park et al. During this process, an azide is required to be carried for multiple steps. Besides safety and stability problems, the azide increases the difficulty of the process by reducing yield and increasing cost. Further, one of the necessary intermediates of the previous process, dimethylphenylpropylamine, is an expensive component. Likewise, the required component homovanillic acid is a compound of limited availability in the absence of specialty manufacturing. Further, multiple chromatographic purifications, which are prohibitively expensive and inefficient under commercial conditions, are required to obtain a sufficient purity or yield of purified product.

As for the reaction itself, it is well-known that the catalytic hydrogenation of a nitrile can be quite difficult. Using typical reducing agents, e.g. high pressure Raney® Nickel (W. R. Grace & Co., New York, N.Y.) hydrogenation or lithium aluminum hydride (LAH) reduction, for conversion of a nitrile to its amine does not provide a high reaction yield. Raney® Nickel additionally requires special equipment for the requisite high pressures and may use highly flammable solvents; the combination of these conditions is both a fire and safety hazard. Further, the LAH reaction is conducted under non-aqueous conditions, frequently using ether solvents. Conversion of nitrites with strong reducing agents, such as aluminum or boron reducing agents, e.g. LAH, are also known to have even required the use of earthen reaction bunkers due to significant exothermic reactions, a hazard not unappreciated by synthetic chemists.

Several solutions to these problems have been unsuccessfully proposed in the art. For example, Caddick et al., in Tetrahedron Letters, 59 (2003) pp. 5417-5423, discuss the commercial availability of a broad range of nitrites, the large number of applications in synthetic chemistry for conversion of nitriles to amines over the years, and the difficulty of reducing a nitrile group with metal hydrides, including the use of nickel and cobalt borohydrides. Caddick et al. also report the use of nickel chloride with excess sodium borohydride to facilitate the production of Boc-protected amines.

Similarly, U.S. Pat. No. 5,869,653 discloses a process for the hydrogenation of nitrites to produce primary amines. In the catalytic hydrogenation of aliphatic nitrites, the nitrile is contacted with hydrogen in the presence of a sponge or Raney® cobalt catalyst employing lithium hydroxide as a promoter. A wide variety of aliphatic nitriles (C₂-C₃₀) are suggested as being suited for conversion to the primary amine by reaction with hydrogen.

U.S. Pat. No. 5,847,220 discloses a process for the catalytic hydrogenation of a cyanopropionaldehyde alkyl acetal in the presence of a nickel or cobalt catalyst promoted with alkali metal hydroxide to form aminobutyraldehyde alkyl acetals, i.e., the primary amine derivative of the cyanoalkyl acetals.

Additionally, U.S. Pat. No. 5,894,074 discloses a process for the preparation of tertiary amines from nitrites and amines utilizing a palladium catalyst and incorporating small amounts of calcium oxide, alumina, magnesium oxide, etc., resided in the inclusion of a small amount of at least one further metal selected from the group of Group IB and Group VIII metals, as well as cerium and lanthanum on a support. However, these proposed solutions do not adequately solve the difficulties noted above.

Another difficulty of the known processes is the particular solvent which is required. One of the most likely solvents, DMF, generates environmental problems and is thermally unstable. Environmental waste problems also arise from the use of LAH since the reaction by-products are alumina sludge and hydrogen gas.

Additional difficulties with the prior art processes which are addressed by the preferred embodiments herein include the problematic creation of aziridines during conversion of a nitrile to an amine, and the problematic choice of the proper temperature which can dictate whether a nitrile-amine reaction will work or will not work.

Use of other standard reduction materials is also not straight-forward. Reductions using other lithium compounds such as LiAlH(OtBu)₃ will not work to reduce a nitrile to an amine in this context. Boron compounds are similarly problematic and selective. For example, BH₃SMe₂ can be effective, but poses problems since it releases dimethyl sulfide and requires a scrubber system to deal with environmental problems.

Additionally, once a target amine is made, storage and stability issues frequently arise. One such example is the development of polymorphs.

The polymorphic behavior of drugs can be of crucial importance in pharmacy and pharmacology. Polymorphs are, by definition, crystals of the same molecule having different physical properties as a result of the order of the molecules in the crystal lattice. The differences in physical properties exhibited by polymorphs can affect pharmaceutical parameters such as storage stability, compressibility and density (important in formulation and product manufacturing), and dissolution rates (an important factor in determining bio-availability). Differences in stability can result from changes in chemical reactivity (e.g. differential oxidation, such that a dosage form discolors more rapidly when comprised of one polymorph than when comprised of another polymorph) or mechanical changes (e.g. tablets crumble on storage as a kinetically favored polymorph converts to thermodynamically more stable polymorph) or both (e.g. tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing: for example, one polymorph might be more likely to form solvates or might be difficult to filter and wash free of impurities (i.e. particle shape and size distribution might be different between one polymorph relative to the other).

Every pharmaceutical compound has an optimal therapeutic blood concentration and a lethal concentration. The bio-availability of the compound determines the dosage strength in the drug formulation necessary to obtain the ideal blood level. If the drug can crystallize as two or more polymorphs differing in bio-availability, the optimal dose will depend on the polymorph present in the formulation. Some drugs show a narrow margin between therapeutic and lethal concentrations. Chloramphenicol-3-palmitate (CAPP), for example, is a broad spectrum antibiotic known to crystallize in at least three polymorphic forms and one amorphous form. The most stable form, A, is marketed. The difference in bio-activity between this polymorph and another form B, is a factor of eight, creating the possibility of fatal overdosages of the compound if unwittingly administered as form B due to alterations during processing and/or storage.

Accordingly, regulatory agencies, such as the U.S. Food and Drug Administration, have begun to place tight controls on the polymorphic content of the active component in solid dosage forms. In general, for drugs that exist in polymorphic forms, if anything other than the pure thermodynamically preferred polymorph is to be marketed, the regulatory agency will require batch-by-batch monitoring. Thus, it becomes important for both medical and commercial reasons to produce and market the most thermodynamically stable polymorph, substantially free of other kinetically favored polymorphs.

From the thermodynamic perspective, only one polymorph will be stable: the one with the lowest free energy at a given temperature and pressure. From the industrial crystallization point of view, however, thermodynamic stability is not sufficient to ensure that the stable polymorph will always be produced. For example, during primary nucleation, in the absence of seed crystals, the unstable polymorph or pseudo polymorph in the form of a hydrate or solvate tends to crystallize first (kinetic form). This is, in essence, Ostwald's Rule of Stages, which posits that an unstable system does not transform directly to the most stable state. Instead, it transforms to a transient state accompanied by the smallest loss of free energy. The eventual transition(s) to the most stable phase is inevitable but the transformation can be extremely fast or extremely slow depending on the process conditions present. Such transformations can occur as a result, and due to, various conditions, including for example grinding, temperature, humidity, and pressure. Some polymorphic transformations can be reversible when the relative solubilities of the polymorphs invert over a range of temperatures (enantiotropic). Other transformations are irreversible (monotropic) over a broad range of temperatures.

Although 2-[4-(2-aminoethoxy)-3-methoxyphenyl]-N-[3-(3,4-dimethylphenyl)propyl]acetamide (DA-5018) is described in the literature, polymorphism of the solid product from these processes is not disclosed.

Accordingly, the present subject matter is believed to solve one or more of the aforementioned problems and to provide improved processes for synthesizing capsaicinoids, especially burnless capsaicin-like compounds such as DA-5018, as well as their polymorphs.

Further, during development of the present improved synthetic process for the reduction of the capsaicinoid nitriles, it was also discovered that an improved process for the catalytic hydrogenation of a nitrile, generally, can afford a benefit to other technical areas in chemistry which have similar nitrile-related problems, for example certain polymer syntheses and certain antibiotic syntheses.

### SUMMARY OF THE INVENTION

Accordingly, the present subject matter provides unique solid forms of 2-[4-(2-aminoethoxy)-3-methoxyphenyl]-N-[3-(3,4-dimethylphenyl)propyl]acetamide (DA-5018), which exists as one of four polymorphic forms or one hydrated form. Accordingly, a polymorph or a hydrate of DA-5018 is contemplated herein. In this regard, substantially pure polymorphs of each of Forms I, II, IV, and V of DA-5018, and a substantially pure dihydrate of form III of DA-5018, are contemplated herein. In a particularly preferred embodiment, a substantially pure polymorph of Form II of DA-5018, as the most favored stable polymorph and the most favored stable solid form, is contemplated herein.

In another preferred embodiment, the present subject matter relates to a crystalline solid comprising at least 95% of a stable polymorph (hereinafter referred to as polymorph II) defined by its X-ray powder diffraction pattern (including both characteristic peaks and intensities).

In yet another preferred embodiment, the present subject matter relates to processes for producing polymorph II of crystalline DA-5018. A preferred process for producing polymorph Form II of crystalline DA-5018 comprises:

i) dissolving crude DA-5018 in an appropriate solvent to obtain a solution;

ii) filtering the solution of step i) to obtain a filtrate;

iii) treating the filtrate with activated carbon to obtain an activated carbon mixture;

iv) filtering the activated carbon mixture and obtaining a residue therefrom;

v) suspending the residue in an appropriate solvent or mixture of solvents to obtain a suspension;

vi) heating the suspension until a heated solution is obtained;

vii) allowing the heated solution to cool over time and a product to crystallize to form a second suspension;

viii) filtering the second suspension to obtain a filter-cake;

ix) washing the filter-cake; and

x) drying the filter-cake to obtain purified DA-5018 polymorph Form II.

In a particularly preferred embodiment, the solvent used in this process is selected from the group consisting of isopropyl acetate, ethyl acetate, methanol, ethanol, acetonitrile, water, and mixtures thereof.

Pharmaceutical compositions are also included as within the scope of the present preferred embodiments. In a particularly preferred embodiment, the pharmaceutical compositions comprise the product prepared by the processes herein, and a pharmaceutically acceptable carrier.

In another preferred embodiment, the present subject matter relates to methods of treating a skin disorder comprising the step of administering to a patient in need thereof an effective amount of the pharmaceutical compositions presented herein. In particular, the treatment of skin disorders selected from the group consisting of post-herpetic neuralgia, pruritis, pruritis associated with atopic dermatitis, acne, atopic dermatitis, and psoriasis is contemplated herein.

In an alternative preferred embodiment, the pharmaceutical compositions are used as vanilloid receptor agonists (VR1) and can be used in methods of treating the diseases and disorders associated therewith, which are well known in the art and are disclosed in, e.g., U.S. Pat. Nos. 6,476,076 and 6,723,720, the contents of which are hereby incorporated by reference in their entirety. In a preferred aspect, the active compound present in these compositions is a vanilloid receptor agonist (VR1) having a Ki of less than about 100 nM in a standardized Ca++ uptake assay. More preferably, these compounds are useful wherein the compound is a vanilloid receptor agonist (VR1) having a receptor binding affinity Ki of less than about 10 nM in a standardized Ca++ uptake assay.

In another aspect, the compounds useful as vanilloid receptor agonists (VR1) have an ED₅₀ of less than about 100 nM in a standardized writhing model, and more preferably an ED₅₀ of less than about 10 nM in a standardized writhing model.

Yet another alternative preferred embodiment of the present subject matter relates to a novel, generalized process for reducing a nitrile to obtain an amine compound, the process comprising the step of catalytically hydrogenating a nitrile compound in a dipolar aprotic organic solvent in the presence of a palladium/carbon catalyst and a strong anhydrous protic acid to obtain an amine compound.

In a more preferred aspect of this embodiment, the dipolar aprotic organic solvent used in the hydrogenation process is selected from the group consisting of THF, NMP, DMF, DMSO, sulfolane, and mixtures thereof; and wherein the strong anhydrous protic acid concentration is from about 0.1 molar eq. to about 10 molar eq. and is selected from the group consisting of trifluoroacetic acid, sulfuric acid, alkylsulfonic acid, arylsulfonic acid, phosphoric acid, alkylphosphoric acid, arylphosphoric acid, and mixtures thereof. Further, the hydrogenation is preferably carried out at a reaction temperature of from about 0° C. to about 10° C., and the palladium/carbon catalyst has a concentration of from about 0.1% to about 20% palladium on carbon. This process is preferably carried out at a hydrogen pressure of from about 10 psig to about 100 psig.

Still yet another preferred embodiment of the present subject matter relates to a process of preparing an amine compound, which comprises: 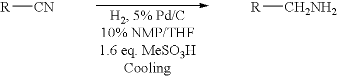
wherein the NMP/THF mixture is anhydrous, and wherein R—CN is a nitrile-containing compound subjected to reduction to provide the amine end product R—CH₂NH₂, and wherein R is any organic compound.

In another preferred embodiment, the present subject matter relates to a process for preparation of a capsaicinoid derivative, which comprises: catalytically hydrogenating a nitrile intermediate compound of Formula Ia: 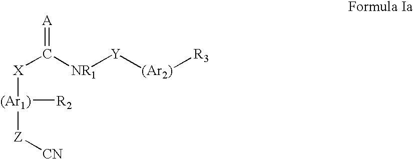
in a dipolar aprotic organic solvent in the presence of a palladium/carbon catalyst and a strong anhydrous protic acid, and obtaining a capsaicinoid derivative, wherein:

X, Y, Z, A, R₁, Ar₁, R₂, Ar₂, and R₃, are defined as set forth herein.

In still another preferred embodiment, the present subject matter relates to nitrile intermediate compounds, especially those of formula Ia which produce a preferred subgenus of final capsaicinoid amines.

Yet another preferred embodiment of the present subject matter relates to amine final products prepared by the processes disclosed herein, especially those processes affording high yield and high levels of purity of the final product, and more especially where the amine final product is DA-5018, 2-[4-(2-aminoethoxy)-3-methoxyphenyl]-N-[3-(3,4-dimethylphenyl)propyl]acetamide as described by the formula:

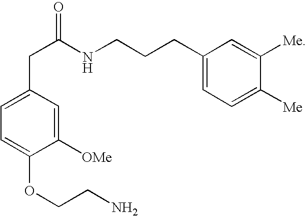

Still another preferred embodiment of the present subject matter relates to a process for preparation of DA-5018, which comprises: catalytically hydrogenating a nitrile compound of the formula:

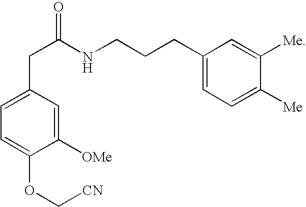

Yet another preferred embodiment of the present subject matter relates to a process for preparing an amine compound, which comprises: catalytically hydrogenating a nitrile compound of the formula: 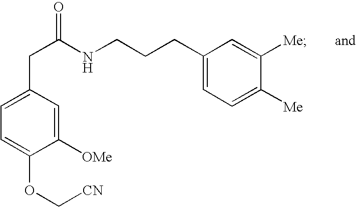
and obtaining an amine product of the formula:

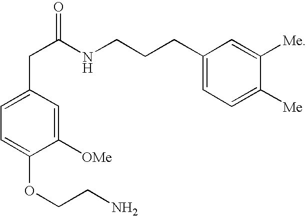

Still another preferred embodiment of the present subject matter relates to a compound which is useful in the manufacture of capsaicinoids, which comprises:

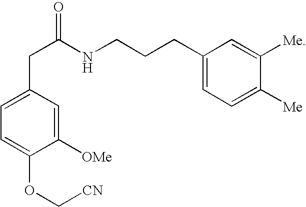

Another preferred embodiment of the present subject matter relates to a process for preparation of an amine product, which process comprises:

deprotecting an intermediate compound of Formula II:

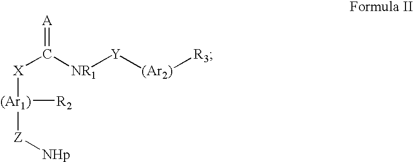

and obtaining an amine product,

wherein X, Y, Z, A, R₁, Ar₁, R₂, Ar₂, and R₃, are defined as set forth herein, and wherein p is a protecting group.

Yet another preferred embodiment of the present subject matter relates to a process for preparation of DA-5018, which comprises:

1) deprotecting an intermediate compound of Formula III: 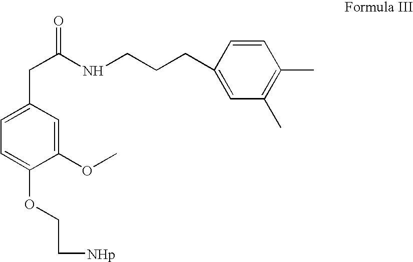
wherein p is a protecting group; and

2) obtaining DA-5018, especially DA-5018 having a high purity.

In still yet another preferred embodiment, the present subject matter relates to additional novel intermediates which are useful in the instant processes for constructing the larger protected amines. These intermediates include a compound of Formula IV, which comprises:

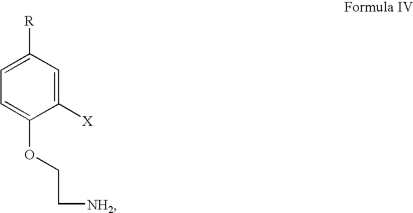

wherein

R is C₁₋₆ alkyl or C₂₋₆ alkenylene substituted with COOH or CONH_{2,} and

X is C₁₋₁₀ alkoxy, C₂₋₁₀ alkenoyl, or C₂₋₁₀ alkenoxy,

with the proviso that R cannot be C₁—COOH when X=methoxy when claimed as a novel compound.

Still another preferred embodiment of the present subject matter relates to another intermediate compound which is useful in the manufacture of capsaicinoids, comprising:

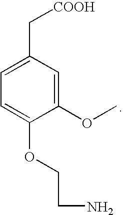

Another preferred embodiment of the present subject matter relates to another intermediate compound which is useful in the manufacture of capsaicinoids, comprising: 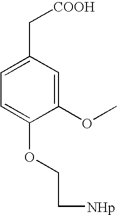
wherein p is a protecting group.

Yet another preferred embodiment of the present subject matter relates to a further intermediate compound which is useful in the manufacture of capsaicinoids, comprising: 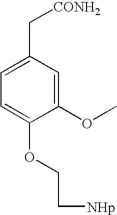
wherein p is a protecting group, as described herein.

Still yet another preferred embodiment of the present subject matter relates to another novel intermediate compound which is useful in the manufacture of capsaicinoids, comprising: 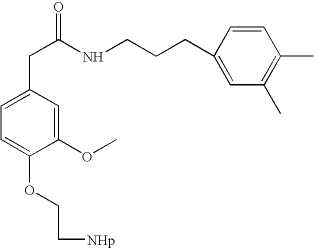
wherein p is a protecting group, as described herein.

A further preferred embodiment of the present subject matter relates to a novel process for preparing an amine compound comprising:

deprotecting Compound A: 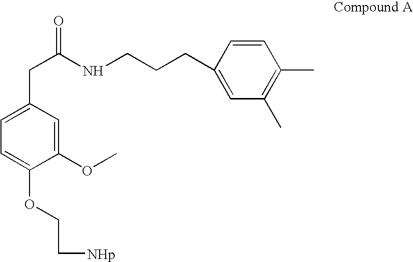
wherein p is a protecting group, as described herein; and

obtaining an amine product, Compound B (DA-5018):

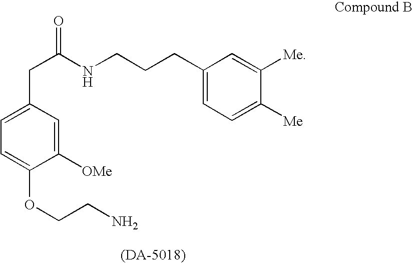

The amine product produced according to this process is preferably at least about 85% pure. In a particularly preferred embodiment, the amine product produced according to this process is preferably at least about 90% pure. In a most preferred embodiment, the amine product produced according to this process is preferably at least about 95% pure.

### BRIEF DESCRIPTION OF THE FIGURES

FIG. 1 is an X-ray powder diffraction (XRPD) stacked plot of unique polymorph and hydrate forms observed.

FIG. 2 shows results from thermal stress experiments, demonstrating conversion of Form I of DA-5018 to Form II:

a.) 2° C./min DSC

b.) 10° C./min DSC

c.) 20° C./min DSC

d.) Isothermal heat-cool-heat DSC

e.) Original XRPD

f.) XRPD after 105° C. for 10 minutes.

FIG. 3 shows results from thermal stress experiments, demonstrating conversion of Form IV of DA-5018 to Form II:

a.) 2° C./min DSC

b.) 10° C./min DSC

c.) 20° C./min DSC

d.) Isothermal heat-cool-heat DSC

e.) Original XRPD

f.) XRPD after melting with Bunsen burner

g.) ¹H NMR spectrum.

FIG. 4 shows a moisture sorption analysis (DVS) of DA-5018 Form I.

FIG. 5 shows a moisture sorption analysis (DVS) of DA-5018 after thermal conversion to Form II.

FIG. 6 is a HPLC plot of a sample of DA-5018.

FIG. 7 is a HPLC chromatogram of a sample of DA-5018.

FIG. 8 is an XRPD chromatogram of a sample of DA-5018.

### DETAILED DESCRIPTION OF THE INVENTION

### Definitions

The term “ACN” as used herein is a term known in the art and refers to the solvent acetonitrile.

The term “alkenylene” as used herein refers to a branched or unbranched unsaturated hydrocarbon chain comprising a designated number of carbon atoms. For example, C₂-C₆ straight or branched alkenyl hydrocarbon chain contains 2 to 6 carbon atoms having at least one double bond, and includes but is not limited to substituents such as ethenyl, propenyl, iso-propenyl, butenyl, iso-butenyl, n-pentenyl, n-hexenyl, and the like. The term “alkenylene” is further intended to encompass alkenyl radicals, as used herein.

The term “alkoxy” as used herein refers to the group —OR wherein R is alkyl as herein defined. Preferably, R is a branched or unbranched saturated hydrocarbon chain containing 1 to 6 carbon atoms.

The term “alkyl” as used herein refers to a branched or unbranched saturated hydrocarbon chain comprising a designated number of carbon atoms. For example, C₁-C₆ straight or branched alkyl hydrocarbon chain contains 1 to 6 carbon atoms, and includes but is not limited to substituents such as methyl, ethyl, propyl, iso-propyl, butyl, iso-butyl, tert-butyl, n-pentyl, n-hexyl, and the like. The term “alkyl” is further intended to encompass alkylene radicals, as used herein.

The term “alkynylene” as used herein refers to a branched or unbranched unsaturated hydrocarbon chain comprising a designated number of carbon atoms. For example, C₂-C₆ straight or branched alkenyl hydrocarbon chain contains 2 to 6 carbon atoms having at least one triple bond, and includes but is not limited to substituents such as ethynyl, propynyl, iso-propynyl, butynyl, iso-butynyl, n-pentynyl, n-hexynyl, and the like. The term “alkynylene” is further intended to encompass alkynyl radicals, as used herein.

The term “amine” as used herein takes the meaning commonly known to a person of ordinary skill in the art, and refers to primary, secondary, and tertiary amines which can be produced according to the processes described herein, most preferably primary amines (R—CH₂NH₂), and in a more preferred aspect, 2-[4-(2-aminoethoxy)-3-methoxyphenyl]-N-[3-(3,4-dimet-hylphenyl)propyl]acetamide (DA-5018).

Particularly preferred amine products that can be produced according to the present processes include alkylamines, arylamines, ethylenediamines, thioethylamines, oxyethylamines, and mixtures thereof.

The “amines” produced by the preferred embodiments of the present processes can be used to produce amine products having a wide variety of practical utilities, including for example, pharmaceuticals, pesticides, herbicides, propellants, polymers, reagents, preservatives, fungicides, fumigants, plant growth regulators, insecticides, drug modifiers, PEG-ylated compounds, and/or intermediates of any of the foregoing. The creation of amine products having other practical utilities that can be formed according to the novel processes herein are further contemplated as within the scope of the preferred embodiments. Specific examples of such compounds where the present advantageous nitrile reduction is expected to facilitate the production are exemplified in further detail below.

The phrase “and mixtures thereof” as used herein refers to a mixture of one or more of the listed agents.

The term “anhydrous” as used herein takes the meaning ordinarily known to a person of ordinary skill in the art and refers to a substantially water-free, or ‘dry’, solution.

The term “aprotic” as used herein refers to an atom or molecule that will neither donate nor accept a proton.

The term “capsaicinoid” as used herein refers broadly to acetamide compounds made from compound intermediates of Formulae I and II.

The term “catalyst” or “catalytically” is intended to be used herein in the usual sense as known by a person of ordinary skill in the art, and refers to the ability of a compound to facilitate a reaction by speeding it up, lowering the energy levels necessary for the reaction to occur, enhancing the yield, enhancing the purity of the products, reducing the amount of starting materials, or the like.

The term “DA-5018” as used herein refers to the specific compound 2-[4-(2-aminoethoxy)-3-methoxyphenyl]-N-[3-(3,4-dimethylphenyl)propyl]acetamide.

The phrase “dipolar aprotic organic solvent” as used herein refers to solvents that do not contain active hydrogen protons. Non-limiting examples of such dipolar aprotic organic solvents include DMA, HMPA, DMPU, THF, NMP, DMF, DMSO, sulfolane, acetonitrile, and mixtures thereof. Additionally, any other dipolar aprotic organic solvents that are carboxamides, lactams, cyclic or acyclic urea derivatives, sulfoxides, sulfones, or equivalents, may be used as is chemically reasonable for the purposes presented herein.

The term “DMA” as used herein refers to N,N-dimethylacetamide, a dipolar aprotic organic solvent.

The term “DMF” as used herein refers to N,N-Dimethylformamide, a dipolar aprotic organic solvent. The term “DMPU” as used herein refers to 1,3-Dimethyl-3,4,5,6-tetrahydro-2(1H)-pyrimidinone, also known as Dimethylpropyleneurea, a dipolar aprotic organic solvent.

The term “DMSO” as used herein refers to Dimethyl Sulfoxide, a dipolar aprotic organic solvent.

The term “enhancing” the biological activity, function, health, or condition of an organism as used herein refers to the process of augmenting, fortifying, strengthening, or improving such biological activity, function, health, or condition.

The term “epithelium” or “epithelial” as used herein refers to the layer of cells forming the epidermis of the skin and the surface layer of mucous and serous membranes. Epithelial cells have the general functions of protection, absorption, and secretion. Epithelial cells are often in close proximity to blood vessels, although generally lacking in a direct blood supply.

The term “EtOAc” as used herein is a term known in the art and refers to the solvent ethyl acetate. This solvent is especially useful herein as a crystallization solvent.

The term “HMPA” as used herein refers to Hexamethylphosphoric triamide, a dipolar aprotic organic solvent.

The term “hydrates” as used herein refers to a specific physical form of a molecule present as a solid crystalline structure containing water molecules bound into, and forming an integral part of, the lattice of the crystal in a likely molar amount, possibly a sub-molar amount. The water molecules are combined in a definite ratio with the crystal. In this regard, “solvates” are contemplated as “hydrates” as used herein.

The term “hydrogenating” or “hydrogenate” as used herein refers to the process of taking a moiety or group which is unsaturated and providing for it to be chemically saturated with hydrogen atoms/molecules, thus reducing any double or triple bonding.

The term “IPA” as used herein is a term known in the art and refers to the solvent isopropanol.

The term “IPAc” as used herein is a term known in the art and refers to the solvent isopropyl acetate, and is synonymous with “iPrOAc”. This solvent is especially useful herein as a crystallization solvent.

The term “isomer” as used herein refers to structurally different compounds that have the same molecular formula. “Stereoisomers” are isomers that differ only in the way the atoms are arranged around a single atom of the molecule. “Enantiomers” are a pair of stereoisomers that are non-superimposable mirror images of each other. “Diastereoisomers” are stereoisomers which are not mirror images of each other. “Racemic mixture” means a mixture containing equal parts of individual enantiomers. A “non-racemic mixture” is a mixture containing unequal parts of individual enantiomers or stereoisomers.

The term “MEK” as used herein is a term known in the art and refers to the solvent methyl ethyl ketone or 2-butanone. This solvent is especially useful herein as a crystallization solvent.

The term “MIBK” as used herein is a term known in the art and refers to the solvent methyl isobutyl ketone or 4-methyl-2-pentanone. This solvent is especially useful herein as a crystallization solvent.

The term “MsOH” as used herein refers to Methanesulfonic acid, or MeSO₃H, a strong anhydrous protic acid.

The term “MTBE” as used herein is a term known in the art and refers to the solvent methyl tert-butyl ether. This solvent is especially useful herein as a crystallization solvent.

The term “NMP” as used herein refers to N-methyl-2-pyrrolidinone, a dipolar aprotic organic solvent.

The term “nitrile” as used herein refers to a chemical moiety or substituent wherein a carbon is triple bonded to a nitrogen, e.g. R—CN. It is also known as a cyano group.

The term “polymorph” as used herein refers to crystals of the same molecule having different physical properties as a result of the order of the molecules in the crystal lattice. Polymorphs of a single compound have different chemical, physical, mechanical, electrical, thermodynamic, and biological properties from each other. The differences in physical properties exhibited by polymorphs affect pharmaceutical parameters such as storage stability, compressibility, density (important in formulation and product manufacturing), dissolution rates (an important factor in determining bio-availability), solubility, melting point, chemical stability, physical stability, powder flowability, compaction, and particle morphology. Differences in stability can result from changes in chemical reactivity (e.g. differential oxidation, such that a dosage form discolors more rapidly when comprised of one polymorph than when comprised of another polymorph) or mechanical changes (e.g. tablets crumble on storage as a kinetically favored polymorph converts to thermodynamically more stable polymorph) or both (e.g. tablets of one polymorph are more susceptible to breakdown at high humidity). As a result of solubility/dissolution differences, some polymorphic transitions affect potency and/or toxicity. In addition, the physical properties of the crystal may be important in processing; for example, one polymorph might be more likely to form solvates or might be difficult to filter and wash free of impurities (i.e. particle shape and size distribution might be different between one polymorph relative to the other).

The phrase “strong anhydrous protic acid” as used herein refers to a hydrogen donating component used in the preferred reactions, and includes as non-limiting examples those selected from the group consisting of perfluoroalkylcarboxylic acid, sulfuric acid, alkylsulfonic acid, arylsulfonic acid, phosphoric acid, alkylphosphoric acid, arylphosphoric acid, pentafluoroalkylcarboxylic acids, hypophosphorous acids, and mixtures thereof.

The term “sulfolane” as used herein refers to tetrahydrothiophene 1,1-dioxide, a dipolar aprotic organic solvent.

The term “TEA” as used herein refers to triethylamine.

The term “THF” as used herein refers to tetrahydrofuran, a dipolar aprotic organic solvent.

The term “treating” as used herein refers to the process of producing an effect on biological activity, function, health, or condition of an organism in which such activity is maintained, enhanced, diminished, or applied in a manner consistent with the general health and well-being of the organism.

### Amine Compounds

The novel processes and intermediates disclosed herein are capable of yielding a variety of amine compounds. Preferred compounds obtained from these novel processes and intermediates include the following.

Capsaicinoid Compounds

In a preferred embodiment, the present processes and intermediates can be used to obtain a capsaicinoid compound of the Formula Ib:

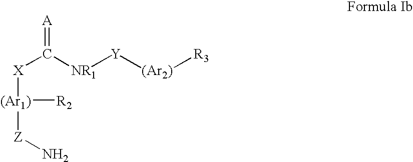

wherein

X is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

Y is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

Z is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

A is oxygen or a sulfur wherein the sulfur is optionally substituted with 2 or 4 hydrogen, oxy, alkyl, alkyloxy, or alkylamino radicals;

R₁ is a hydrogen radical, or an alkyl, alkenylene, or alkynylene, each of which is straight or branched and is radical optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

Ar₁ is a heterocycle, aryl, or heteroaryl radical wherein Ar₁ is substituted in one to five places with R₂;

R₂ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

Ar₂ is a heterocycle, aryl, or heteroaryl radical wherein Ar₂ is substituted in one to five places with R₃;

R₃ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

R₄ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo;

R₅ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo; and

wherein said heterocycle is a radical of a monocyclic or bicyclic saturated heterocyclic ring system having 5-8 ring members per ring, wherein 1-3 ring members are oxygen, sulfur or nitrogen heteroatoms, which is optionally partially unsaturated or benzo-fused and optionally substituted by 1-2 oxo or thioxo radicals; said aryl is a phenyl or naphthyl radical; and said heteroaryl is a radical of a monocyclic or bicyclic aromatic heterocyclic ring system having 5-6 ring members per ring, wherein 1-3 ring members are oxygen, sulfur or nitrogen heteroatoms, which is optionally benzo-fused or saturated C₃-C₄-carbocyclic-fused.

In a preferred embodiment, X is a methyl group. In another preferred embodiment, Y is a propyl group. In still another preferred embodiment, Z is an ethoxy group. In yet another preferred embodiment, A is oxygen. In still yet another preferred embodiment, R₁ is hydrogen. In another preferred embodiment, Ar₁ is phenyl. In still another preferred embodiment, R₂ is a methoxy group.

In another preferred embodiment, Ar₂ is phenyl. In still another preferred embodiment, R₃ is a methyl group. In a particularly preferred embodiment in this regard, R₃ is a methyl group that is substituted at positions 3 and 4 of the Ar₂ heterocycle, aryl, or heteroaryl radical. In an especially preferred embodiment in this regard, Ar₂ is a phenyl group and R₃ is a methyl group substituted at positions 3 and 4 of the phenyl group.

Representative, non-limiting examples of capsaicinoid species falling within generic Formula Ib that can be made according to the processes described herein include phenyl acetamide derivatives:

2-[4-(2-aminoethoxy)-3-methoxyphenyl]-N-[3-(3,4-dimethylphenyl)propyl]acetamide (DA-5018);

N-{3-(3,4-dimethylphenyl)propyl}-4-(2-aminoethoxy)-3-hydroxyphenylacetamide;

N-{2-(3,4-dimethylphenyl)ethyl}-4-(2-aminoethoxy)-3-hydroxyphenylacetamide;

N-(3-phenylpropyl)-4-(2-aminoethoxy)-3-hydroxyphenylacetamide;

N-{3-(3-methylphenyl)propyl}-4-(2-aminoethoxy)-3-hydroxyphenylacetamide;

N-{3-(4-methylphenyl)propyl}-4-(2-aminoethoxy)-3-hydroxyphenylacetamide;

N-{3-(4-chlorophenyl)propyl}-4-(2-aminoethoxy)-3-hydroxyphenylacetamide;

N-{3-(3,4-dichlorophenyl)propyl}-4-(2-aminoethoxy)-3-hydroxyphenylacetamide;

N-{3-(4-fluorophenyl)propyl}-4-(2-aminoethoxy)-3-hydroxyphenylacetamide;

N-{3-(3,4-methylenedioxyphenyl)propyl}-4-(2-aminoethoxy)-3-hydroxyphenylacetamide;

N-{3-(3-methoxyphenyl)propyl}-4-(2-aminoethoxy)-3-hydroxyphenylacetamide;

N-{3-(3-trifluoromethylphenyl)propyl}-4-(2-aminoethoxy)-3-hydroxyphenylacetamide;

N-{3-(3,5-dimethylphenyl)propyl}-4-(2-aminoethoxy)-3-hydroxyphenylacetamide;

N-{3-(3-ethylphenyl)propyl}-4-(2-aminoethoxy)-3-hydroxyphenylacetamide;

N-(4-phenylbutyl)-4-(2-aminoethoxy)-3-hydroxyl-phenyl-acetamide;

N-{4-(3,4-dimethylphenyl)butyl}-4-(2-aminoethoxy)-3-hydroxyphenylacetamide;

N-(5-phenylpentyl)-4-(2-aminoethoxy)-3-hydroxy-phenylacetamide;

N-{3-(3,4-dimethylphenyl)propyl}-4-(2-aminoethoxy)-3-nitrophenylacetamide;

N-{2-(3,4-dimethylphenyl)ethyl}-4-(2-aminoethoxy)-3-nitrophenylacetamide;

N-(3-phenylpropyl)-4-(2-aminoethoxy)-3-nitro-phenylacetamide;

N-{3-(3-methylphenyl)propyl}-4-(2-aminoethoxy)-3-nitrophenylacetamide;

N-{³-(4-methylphenyl)propyl}-4-(2-aminoethoxy)-3-nitrophenylacetamide;

N-{3-(4-chlorophenyl)propyl}-4-(2-aminoethoxy)-3-nitrophenylacetamide;

N-{3-(3,4-dichlorophenyl)propyl}-4-(2-aminoethoxy)-3-nitrophenylacetamide;

N-{3-(4-fluorophenyl)propyl}-4-(2-aminoethoxy)-3-nitrophenylacetamide;

N-{3-(3,4-methylenedioxyphenyl)propyl}-4-(2-aminoethoxy)-3-nitrophenylacetamide;

N-{3-(3-methoxyphenyl)propyl}-4-(2-aminoethoxy)-3-nitrophenylacetamide;

N-{3-(3-trifluoromethylphenyl)propyl}-4-(2-aminoethoxy)-3-nitrophenylacetamide;

N-{3-(3,5-dimethylphenyl)propyl}-4-(2-aminoethoxy)-3-nitrophenylacetamide;

N-{3-(3-ethylphenyl)propyl}-4-(2-aminoethoxy)-3-nitrophenylacetamide;

N-(4-phenylbutyl)-4-(2-aminoethoxy)-3-nitro-phenylacetamide;

N-{4-(3,4-dimethylphenyl)butyl}-4-(2-aminoethoxy)-3-nitrophenylacetamide;

N-(5-phenylpentyl)-4-(2-aminoethoxy)-3-nitro-phenylacetamide;

N-{3-(3,4-dimethylphenyl)propyl}-4-(2-aminoethoxy)-3-aminophenylacetamide;

N-{2-(3,4-dimethylphenyl)ethyl}-4-(2-aminoethoxy)-3-aminophenylacetamide;

N-(3-phenylpropyl)-4-(2-aminoethoxy)-3-amino-phenylacetamide;

N-{3-(3-methylphenyl)propyl}-4-(2-aminoethoxy)-3-amino-phenylacetamide;

N-{3-(4-methylphenyl)propyl}-4-(2-aminoethoxy)-3-aminophenylacetamide;

N-{3-(4-chlorophenyl)propyl}-4-(2-aminoethoxy)-3-aminophenylacetamide;

N-{3-(3,4-dichlorophenyl)propyl}-4-(2-aminoethoxy)-3-aminophenylacetamide;

N-{3-(4-fluorophenyl)propyl}-4-(2-aminoethoxy)-3-aminophenylacetamide;

N-{3-(3,4-methylenedioxyphenyl)propyl}-4-(2-aminoethoxy)-3-aminophenylacetamide;

N-{3-(3-methoxyphenyl)propyl}-4-(2-aminoethoxy)-3-aminophenylacetamide;

N-{3-(3-trifluoromethylphenyl)propyl}-4-(2-aminoethoxy)-3-aminophenylacetamide;

N-{3-(3,5-dimethylphenyl)propyl}-4-(2-aminoethoxy)-3-aminophenylacetamide;

N-{3-(3-ethylphenyl)propyl}-4-(2-aminoethoxy)-3-aminophenylacetamide;

N-(4-phenylbutyl)-4-(2-aminoethoxy)-3-amino-phenylacetamide;

N-{4-(3,4-dimethylphenyl)butyl}-4-(2-aminoethoxy)-3-aminophenylacetamide;

N-(5-phenylpentyl)-4-(2-aminoethoxy)-3-amino-phenylacetamide;

N-{3-(4-chlorophenyl)propyl}-4-(2-aminoethoxy)-phenylacetamide;

N-{3-(4-chlorophenyl)propyl}-4-(2-aminoethoxy)-3-fluorophenylacetamide;

N-{3-(4-chlorophenyl)propyl}-4-(2-aminoethoxy)-3-chlorophenylacetamide;

N-{3-(4-chlorophenyl)propyl}-4-(2-aminoethoxy)-3-methoxyphenylacetamide;

N-{3-(2,4-dichlorophenyl)propyl}-4-(2-aminoethoxy)-3-methoxyphenylacetamide;

N-{3-(3,4-dichlorophenyl)propyl}-4-(2-aminoethoxy)-3-methoxyphenylacetamide;

N-{3-(4-fluorophenyl)propyl}-4-(2-aminoethoxy)-3-methoxyphenylacetamide;

N-{3-(3,4-methylenedioxyphenyl)propyl}-4-(2-aminoethoxy)-3-methoxyphenylacetamide;

N-{3-(3-methoxyphenyl)propyl}-4-(2-aminoethoxy)-3-methoxyphenylacetamide;

N-{3-(3-benzyloxyphenyl)propyl}-4-(2-aminoethoxy)-3-methoxyphenylacetamide;

N-{3-(3,4-dimethoxyphenyl)propyl}-4-(2-aminoethoxy)-3-methoxyphenylacetamide; and

N-{3-(3-trifluoromethylphenyl)propyl}-4-(2-aminoethoxy)-3-methoxyphenylacetamide.

Further, representative, non-limiting examples of N-arylalkyl-phenylacetamide derivatives of generic Formula Ib that can be made according to the present processes include:

N-{3-(3,4-dimethylphenyl)propyl}-4-(2-hydroxyethoxy)-3-methoxyphenylacetamide;

N-{3-(3,4-dimethylphenyl)propyl}-4-2-{N-(2-hydroxyethyl)}-aminoethoxy-3-methoxyphenylacetamide;

N-{3-(3,4-dimethylphenyl)propyl}-4-2-{N,N-di(2-hydroxyethyl)}-aminoethoxy-3-methoxyphenylacetamide;

N-{3-(3,4-dimethylphenyl)propyl}-4-2-{N-(2-aminoethyl)}aminoethoxy-3-methoxyphenylacetamide;

N-{3-(3,4-dimethylphenyl)propyl}-4-(2-piperazinylethoxy)-3-methoxyphenylacetamide;

N-{3-(3,4-dimethylphenyl)propyl}-4-(2-ethylformamido)-3-methoxyphenylacetamide;

N-{3-(3,4-dimethylphenyl)propyl}-4-{2-(N-benzyloxycarbonyl)-aminoethoxy}-3-methoxyphenylacetamide;

N-{3-(3,4-dimethylphenyl)propyl}-4-(2-methylaminoethoxy)-3-methoxyphenylacetamide;

N-{3-(3,4-dimethylphenyl)propyl}-4-{2-(1-pyrrolidinyl)-ethoxy}-3-methoxyphenylacetamide;

N-{3-(3,4-dimethylphenyl)propyl}-4-{2-(N-ethyloxycarbonyl)aminoethoxy}-3-methoxyphenylacetamide; and

N-{3-(3,4-dimethylphenyl)propyl}-4-2-{N-(2-acetoxybenzoyl)}-aminoethoxy-3-methoxyphenylacetamide.

Additional compounds beyond the phenylacetamides, capsaicinoids, or N-arylalkyl-phenylacetamides described above that can be produced according to the present processes include the following reaction intermediates and/or end-products.

Adiponitrile, is a non life science example which is used in the production of Nylon 6® (available from BASF, Germany). The production of 1,6-hexanediamine by reduction of it's intermediate is a known nitrile reduction wherein the present subject matter is expected to provide a novel, useful, and beneficial improved process.

Alfentanil, CAS 71195-58-9 and its hydrochloride monohydrate salt, known as Rapifen® by Janssen-Cilag or Alfenta® by Janssen, is an analgesic and can have a nitrile intermediate of its 4-(methoxymethyl)-4-piperidinyl-N-phenylpropanamide which is converted to its primary amine before constructing the 4,5-dihydro-5-oxo-1H-tetrazolyl unit.

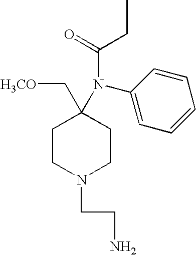

### N-[1-(2-Aminoethyl)-4-methoxymethyl-piperidin-4-yl]-N-phenyl-propionamide

Amidochlor, CAS 40164-67-8, known as Limit® by Monsanto (St. Louis, Mo.), is a plant (turf grasses) growth regulator, and can have an alpha-aminoacetonitrile intermediate of its N-(2,6-diethyl)aniline which is converted to its primary amine.

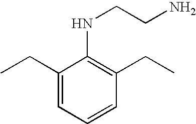

### N¹-(2,6-Diethylphenyl)ethane-1,2-diamine

Fenoxycarb, CAS 72490-01-8, known as Comply® or Insegar® by Syngenta, is an insecticide and can have an intermediate of its (4-phenoxy)phenoxyacetonitrile which can be reduced to its ethylamine.

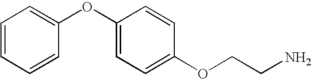

### 2(4-Phenoxyphenoxy)ethylamine

Acecainide, CAS 32795-44-1, known as an antiarrythmic metabolite of procainamide and as a useful synthetic intermediate in its own right can be produced by reduction of N,N-diethylaminoacetonitrile.

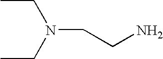

### N¹,N¹-Diethyl-1,2-ethanediamine

N-(1-Naphthyl)ethylenediamine, CAS 551-09-7, known as a reagent useful in the determination of sulfanilamide, potassium, nitrites, and sulfates in blood, can have a nitrile intermediate to form the final product.

Alfuzosin, CAS 81403-80-7, known as the antihypertensive Mittoval® by Schering AG, Urion® by Zambon, or Xatral® by Synthelabo, can have a nitrile intermediate of its quinozolidinyl intermediate.

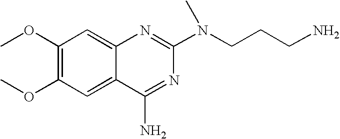

### N²-(3-Aminopropyl) -6,7-dimethoxy-N²-methylquinazoline-2,4-diamine

Bifermalane, CAS 90293-01-9, an antidepressant MAO inhibitor known as Alnert® by Fujisawa or Celeport® by Eisai, can have a nitrile intermediate of its 2-(4-butoxy)diphenylmethane converted to its amine.

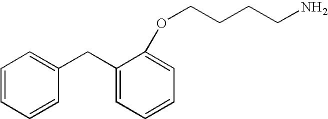

### 4-(2-Benzylphenoxy)butylamine

Carvedilol, CAS 72956-09-3, a nonselective beta-adrenergic blocker with alpha-1 blocking activity, i.e. a antihypertensive, which is known as Coreg® by Smith Kline Beecham, Dilatrend® by Boehringer, and other names, can have a nitrile intermediate of its 2-(2-methoxyphenoxy)ethylamine.

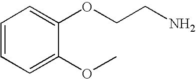

### 2-(2-Methoxyphenoxy)ethylamine

Denopamine, CAS 71771-90-9, a selective beta-adrenoceptor agonist with positive inotropic activity (cardiotonic), which is known as Carguto® or Kalgut® by Tanabe Seiyaku, can have a nitrile intermediate of its 3,4-dimethoxyphenyl intermediate converted to the amine.

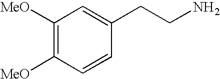

### 2-(3,4-Dimethoxyphenyl)ethylamine

Dofetilide, CAS 115256-11-6, an antiarrythmic (Class III) potassium channel blocker, known as Tikosyn® by Pfizer, can have a nitrile intermediate of its methyl-methylsulfonyl-amino-phenoxy intermediate.

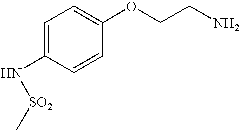

### N-[4-(2-Aminoethoxy)phenyl]methanesulfonamide

Etafenone, CAS 90-54-0, a vasodilator known as Asamedol® by Maruko, Baxacor® by Mack, Corodilan® by Meiji, Dialicor® by Kissei, and other names, can have a nitrile intermediate which converts to the amine.

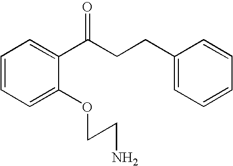

### 1-[2-(2-Aminoethoxy-phenyl]-3-phenyl-propan-1-one

Repinotan, CAS 144980-29-0, a neuroprotective and serotonin 5-HT_{1A} receptor agonist by Bayer AG, can have a nitrile intermediate of its benzopyran converted to the amine.

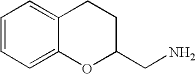

### C-Chroman-2-yl-methylamine

Roxatidine Acetate, CAS 78628-28-1, an antiulcerative histamine H2-receptor antagonist, known as Altat® by Teikoku, Gastralgin® by DeAngeli, NeoH2® by Boehringer, and Roxit® by Aventis, can have a nitrile intermediate of its piperidinylmethyl-phenoxy reduced to its amine.

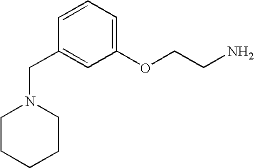

### 2-(3-Piperidin-1-ylmethyl-phenoxy)-ethylamine

Guanethidine sulfate, CAS RN 60-02-6, an antihypertensive, known as Esimil® and Ismelin® by Novartis, and Thilodigon® by Alcon, among others, has a nitrile converted to an amine which is then reacted with the S-methylthiouronium sulfate to provide the final product.

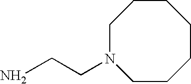

### 2-Azocan-1-yl-ethylamine

These examples of compounds capable of production according to the present processes are considered to be illustrative and non-limiting, and are meant to illustrate that the preferred embodiments are contemplated to be useful across many relevant areas of the particular chemical fields disclosed herein.

### Processes of Preparation

I. Hydrogenation of a Nitrile to Form an Amine

In one aspect, a preferred process herein pertains broadly to a single step process for catalytically hydrogenating a nitrile intermediate compound to produce an amine compound. This process chemistry comprises catalytically hydrogenating the nitrile compound in a dipolar aprotic organic solvent in the presence of a palladium/carbon catalyst and a strong anhydrous protic acid; and obtaining an amine compound. In a preferred embodiment, this process affords from about 50% to about 99% pure amine product. In a particularly preferred embodiment, this process affords from about 85% to about 99% pure amine product. In a most preferred embodiment, this process affords an amine product with over about 85% purity. Further, in preferred embodiments this process has a yield of over about 50%. In a particularly preferred embodiment, this process has a yield of over about 80%. In another particularly preferred embodiment, this process has a yield of over about 85%.

An amine product prepared according to this process is further contemplated herein. This amine product can have a wide variety of practical utilities, including for example, without limitation, pharmaceuticals, pesticides, herbicides, propellants, polymers, reagents, preservatives, fungicides, fumigants, plant growth regulators, insecticides, drug modifiers, PEG-ylated compounds, intermediates thereof, and mixtures thereof.

The single step catalytic hydrogenation process is described by the following equation: 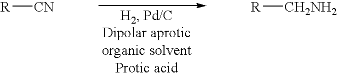
wherein the dipolar aprotic organic solvent is anhydrous, and wherein R—CN is a nitrile-containing intermediate compound subjected to reduction to provide the amine end product R—CH₂NH₂.

Nitrile Reaction Materials

A wide variety of nitrites including, without limitation, substituted and unsubstituted C₂-C₃₀ aliphatic or aromatic nitrites, can be catalytically hydrogenated according to the present process to form an amine product.

Specific, non-limiting examples of nitrites that can be catalytically hydrogenated according to this process include aliphatic nitrites such as acetonitrile, propionitrile, butyronitrile, and valeronitrile; ether nitriles such as ethoxypropionitrile, methoxypropionitrile, isopropoxypropionitrile, biscyanoethylether, bis-(2-cyano-ethyl)ethyleneglycol, bis-(2-cyanoethyl)diethyleneglycol, mono (2-cyanoethyl)diethyleneglycol, and bis(2-cyano-ethyl)tetra-methylene glycol; long chain nitrites such as saturated and unsaturated C₈-C₂₀ fatty alkyl nitrites, e.g., lauronitrile, cocoalkyl nitrile, oleonitrile, tall oil fatty acid nitrile, and stearonitrile; dinitriles such as adiponitrile, methylglutaronitrile, and succinonitrile; α-aminonitriles such as α-aminopropionitrile, di-(2-cyanoethyl)amine, N-methyl-α-aminopropionitrile, N,N-dimethyl-α-aminopropionitrile, N-(2-cyanoethyl) ethanolamine, N,N-di-(2-cyanoethyl)ethanolamine, N-(2-cyanoethyl)diethanolamine, and N-(2-cyanoethyl)propanol amine; α-cyanoethylated amides such as cyanoethylated acetamide and cyanoethylated propionamide; and aromatic nitrites such as α-hydroxybenzene-acetonitrile, benzyl cyanide, benzonitrile, isophthalonitrile, and terephthalo-nitrile.

In a preferred embodiment, each of these nitrile compounds may form the core structure of a nitrile compound subject to the present catalytic hydrogenation process. That is, compounds containing these exemplary nitrile “groups” may be additionally substituted with other groups non-reactive with the process catalyst to form preferred compounds subject to the present process.

In a particularly preferred embodiment of the present subject matter, the nitrile is a nitrile-containing intermediate which is reduced to provide a pharmaceutically useful end product, such as DA-5018.

Palladium/Carbon Catalyst

A critical, novel feature of the present preferred catalytic hydrogenation process is the use of a palladium (Pd) catalyst to achieve the hydrogenation of the nitrile compound. The use of palladium as a catalyst offers an improved selectivity, enhanced reaction rate, and the ability to use reasonable and safe reaction conditions in comparison with other catalytic metals known in the art, such as ruthenium, rhodium, copper, and platinum.

In a preferred embodiment of the present hydrogenation process, the palladium catalyst can be carried upon a heterogeneous carbon support for ease of removal from the reaction medium. Other possible supports for the present catalyst can include alumina, silica, barium salts, organic polymer, resin (plastics), and the like. According to this embodiment, the catalyst preferably has a concentration of from about 0.1% to about 20% palladium on carbon. In a particularly preferred aspect, the catalyst is a readily available non-specific 10% palladium on carbon (50% wet paste).

In an alternative preferred embodiment, the catalyst has a concentration of about 5% palladium on carbon (50% wet paste).

In another preferred embodiment, the palladium/carbon catalyst is in suspension or is a dispersion. According to this embodiment, the palladium/carbon catalyst is in suspension so that it preferably has a catalyst loading of about 0.1% to about 50% by weight. In a particularly preferred embodiment, the palladium/carbon catalyst has a catalyst loading of about 5% to about 20% by weight.

Dipolar Aprotic Organic Solvents

The palladium/carbon catalyst is preferably present in a dipolar aprotic organic solvent to allow the reaction to proceed. Preferred, non-limiting examples of dipolar aprotic organic solvents useful in the present processes include DMA, HMPA, DMPU, THF, NMP, DMF, DMSO, sulfolane, and mixtures thereof. In a particularly preferred embodiment, the dipolar aprotic organic solvent is selected from the group consisting of THF, NMP, DMF, DMSO, sulfolane, and mixtures thereof. Additionally, any other dipolar aprotic organic solvents that are carboxamides, lactams, cyclic or acyclic urea derivatives, sulfoxides, or sulfones may also be used according to the present processes.

In a preferred embodiment, the dipolar aprotic organic solvent is a mixture of from about 0.1% to about 30% NMP in THF. In a particularly preferred embodiment, the dipolar aprotic organic solvent is about 10% NMP in THF.

Strong Anhydrous Protic Acids

A key to the effectiveness of the present process is the presence of a strong anhydrous protic acid during the catalytic hydrogenation of the nitrile compound. The preferred anhydrous protic acids used in this regard have a PKₐ of less than or equal to about 2 relative to water. Further, the strong anhydrous protic acids are preferably present during the catalytic reaction in a concentration of about 0.1 molar eq. to about 10 molar eq. In a particularly preferred embodiment, the strong anhydrous protic acid is present during the catalytic reaction at a concentration of about 1.6 molar eq., based on the number of amines in the product. For example, 1 amine would require 1.6 molar equivalents per basic amine, 2 amines would require 3.2 molar equivalents, and so forth.

Preferred, non-limiting examples of strong anhydrous protic acids useful according to the present process include those selected from the group consisting of sulfuric acid, alkylsulfonic acids, arylsulfonic acids, phosphoric acid, alkylphosphoric acids, arylphosphoric acids, perfluoroalkyl carboxylic acids, hypophosphorous acids, and mixtures thereof.

In a preferred embodiment, non-limiting examples of perfluoroalkylcarboxylic acids useful as strong anhydrous protic acids herein include those perfluoroalkylcarboxylic acids containing a C₁-C₂₀ alkyl group. Particularly preferred in this regard is trifluoroacetic acid.

Preferred, non-limiting examples of alkylsulfonic and alkylphosphoric acids useful as strong anhydrous protic acids herein include those having an alkyl group selected from the group consisting of C₁-C₂₀ alkyl, C₃-C₂₀ cycloalkyl, C₃-C₂₀ unsaturated carbocycle, and mixtures thereof. Particularly preferred in this regard is methanesulfonic acid (MsOH or MeSO₃H).

Preferred, non-limiting examples of arylsulfonic and arylphosphoric acids useful as strong anhydrous protic acids herein include those having an aryl group selected from the group consisting of a heterocycle radical, an aryl radical, a heteroaryl radical, and mixtures thereof. The heterocycle radical can be a monocyclic or bicyclic saturated heterocyclic ring system having 5-8 ring members per ring, wherein 1-3 ring members of each ring are oxygen, sulfur or nitrogen heteroatoms, which is optionally partially unsaturated or benzo-fused and optionally substituted by 1-2 oxo or thioxo radicals. Similarly, the aryl radical can be a phenyl or naphthyl radical; and the heteroaryl radical can be a radical of a monocyclic or bicyclic aromatic heterocyclic ring system having 5-6 ring members per ring, wherein 1-3 ring members of each ring are oxygen, sulfur or nitrogen heteroatoms, which is optionally benzo-fused or saturated C₃-C₄-carbocyclic-fused.

In a particularly preferred embodiment, the strong anhydrous protic acid is selected from the group consisting of trifluoroacetic acid, methanesulfonic acid, sulfuric acid, and mixtures thereof. In a most preferred embodiment, the strong anhydrous protic acid is methanesulfonic acid or sulfuric acid and the strong anhydrous protic acid has a concentration of about 1.6 molar eq. based on the number of nitrogens in the molecule.

Reaction Conditions—Temperature and Pressure

The present processes are unique and novel in that they permit the catalytic hydrogenation of nitrile compounds to form corresponding amine compounds using reasonable, typical, and safe reaction conditions, e.g. temperature and pressure. Accordingly, the present catalytic hydrogenation processes provide distinct advantages over those known in the art in that they are more economical and less dangerous to amine producers. Further, the present processes make it easier to prepare amine products than according to those processes previously known in the art.

For example, by using a palladium/carbon catalyst to catalytically reduce a nitrile compound, the present processes can be carried out at a preferred reaction temperature of from about −10° C. to about 25° C. In a particularly preferred embodiment, the reaction temperature is about 0° C. to about 10° C. These reaction temperatures permit the present processes to be carried out in standard hydrogenation equipment, without the need for extreme safety precautions necessary for many prior art processes.

Similarly, the novel features of the present processes permit these processes to be carried out at a hydrogen pressure of about 5 psig to about 300 psig. In a preferred embodiment, the hydrogen pressure is about 10 psig to about 100 psig. In a particularly preferred embodiment, the hydrogen pressure is about one atmosphere or about 16 psig, with a range from about 14 to about 20 psig, especially when the reaction vessel is glass or glass lined. In another particularly preferred embodiment, the hydrogen pressure is about 50 psig. Again, the use of such a hydrogen pressure permits the present processes to be carried out in standard equipment, without the need for the extreme safety precautions necessary for many prior art processes.

Process Intermediates: Cyano-Capsaicinoids

Exemplary nitrile intermediate compounds of formula Ia, below, are useful in the preparation of amine products, particularly final capsaicinoids of the preferred embodiments of the present subject matter:

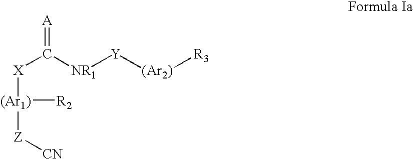

wherein:

X is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

Y is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

Z is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

A is oxygen or a sulfur wherein the sulfur is optionally substituted with 2 or 4 hydrogen, oxy, alkyl, alkyloxy, or alkylamino radicals;

R₁ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

Ar₁ is a heterocycle, aryl, or heteroaryl radical wherein Ar₁ is substituted in one to five places with R₂;

R₂ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

Ar₂ is a heterocycle, aryl, or heteroaryl radical wherein Ar₂ is substituted in one to five places with R₃;

R₃ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

R₄ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo;

R₅ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo; and

wherein said heterocycle is a radical of a monocyclic or bicyclic saturated heterocyclic ring system having 5-8 ring members per ring, wherein 1-3 ring members are oxygen, sulfur or nitrogen heteroatoms, which is optionally partially unsaturated or benzo-fused and optionally substituted by 1-2 oxo or thioxo radicals; said aryl is a phenyl or naphthyl radical; and said heteroaryl is a radical of a monocyclic or bicyclic aromatic heterocyclic ring system having 5-6 ring members per ring, wherein 1-3 ring members are oxygen, sulfur or nitrogen heteroatoms, which is optionally benzo-fused or saturated C₃-C₄-carbocyclic-fused.

Exemplary nitrile intermediate compounds of formula Ia which produce a preferred subgenus of final capsaicinoid amines, include those wherein:

X is a C₁₋₁₀ alkyl or C₂₋₁₀ alkenylene radical;

Y is a C₁₋₂₀ alkyl or C₂₋₁₀ alkenylene radical;

Z is a C₁₋₂₀ alkyl, C₁₋₂₀ alkyloxy, C₂₋₂₀ alkenylene, or C₂₋₂₀ alkenoxy radical;

A is oxygen or sulfur;

R₁ is hydrogen, C₁₋₂₀ alkyl, or C₂₋₂₀ alkenylene;

Ar₁ is a C₃₋₂₀ carbocyclic ring or C₃₋₂₀ heterocyclic ring having one or more heteroatoms selected from O, N, or S, wherein Ar₁ is substituted in one to five places with R₂;

R₂ is hydrogen, C₁₋₂₀ alkyl, C₂₋₂₀ alkenylene, C₁₋₂₀ alkyloxy, C₂₋₂₀ alkenoxy, C₁₋₂₀ thioalkyl, or C₂₋₂₀ thioalkenylene;

Ar₂ is a C₃₋₂₀ carbocyclic ring or C₃₋₂₀ heterocyclic ring having one or more heteroatoms selected from O, N, or S, wherein Ar₂ is substituted in one to five places with R₃;

R₃ is hydrogen, C₁₋₂₀ alkyl, C₂₋₂₀ alkenylene, C₁₋₂₀ alkyloxy, C₂₋₂₀ alkenoxy, C₁₋₂₀ thioalkyl, or C₂₋₂₀ thioalkenylene.

II. Hydrogenation Synthesis—Preparation of DA-5018 (1)

In a preferred embodiment, the present processes are used to synthesize DA-5018, which preferably can be synthesized in kilogram quantities. The synthetic scheme below shows one preferred synthetic approach to prepare DA-5018. It is important to note that material throughput has been significantly increased in the early steps of the synthesis. Suppliers of the chemical reagents are easily found or reagents are manufactured according to the knowledge of persons of ordinary skill in the chemical synthesis field.

Key Synthetic Aspects

The key aspects of the synthesis are summarized as follows:

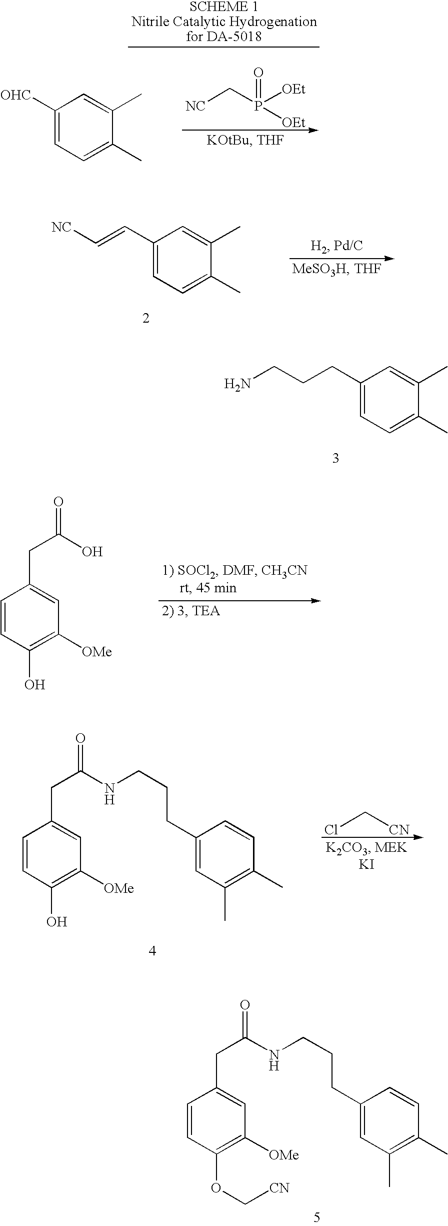

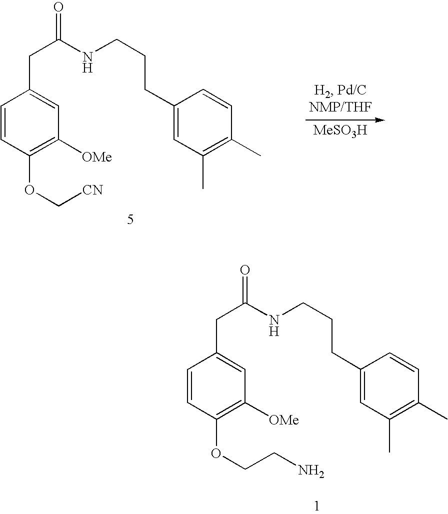

Step 1: HWE (Horner-Wadsworth-Emmons methodology) chemistry provides a facile, scaleable preparation of acrylonitrile 2. The yield is essentially quantitative and the purity is excellent. These factors increase throughput and provide high-purity material in comparison to the alternately employed chemistry.

Step 2: Catalytic hydrogenation of acrylonitrile 2 is also a more facile, scaleable, and safe approach to amine 3 compared to certain previous approaches, for example such as the two-step hydrogenation sequence in the prior art, e.g. U.S. Pat. Nos. 5,242,944 and 5,670,546 to Park et al. The hydrogenation is a clean reaction generating minimal byproducts. Storage can be an issue for this amine. However, preparation of the hydrochloride salt of the amine is a convenient method of purification and storage for this amine. Furthermore, the amine free base can be easily regenerated from the hydrochloride salt as a toluene solution suitable for use in the subsequent reaction.

Step 3: The coupling of amine 3 and homovanillic acid can be a troublesome step in synthesis. Development work showed that the product purity can be controlled by careful pH adjustments during work-up.

Step 4: Alkylation of 4 to afford nitrile 5 can be more difficult than anticipated. The key factor in this chemistry is the quality of phenol 4 used in the alkylation. Chromatographed phenol 4 gave 5 in >90% yield and 99% purity (AUC) in 20 hours of reaction time with no purification required. When lower purity phenol 4 was used, these results were not achieved. The reaction of 4 with chloroacetonitrile was slow and additional time, base, and alkylating agent were required. The introduction of potassium iodide improved the reaction, resulting in lower reagent loading, lower reaction temperature, and shorter reaction time.

Step 5: Hydrogenation of 5 to DA-5018 1 was successful on a kilogram scale. The key reaction parameters were determined to be the reaction dilution (17 volumes solvent/acid mixture), stirring rate, and temperature control. If these parameters are not used, cyanohydrin 5 will decompose to phenol 4.

Analytical/In-Process Control

The reactions for the subject matter discussed herein were mainly followed by HPLC. The following is a description of the instrument and column used.

HPLC analysis was performed on a Varian Prostar with UV detection. The solvent system used was a mixture of solvent A: water with 0.1% TFA by volume and solvent B: acetonitrile with 0.1% TFA by volume. The purity % is reported as the area under the curve (AUC).

Column: Hypersil BDS C18

Length: 150 mm

Diameter: 4.6 mm

Pore Size: 5 μm

Flow Rate: 1 mL/minute

Injection Volume: 5 μL

Method A: Detector: 254 nm

| | 0.1% TFA in Water | 0.1% TFA in CH₃CN |
|---|---|---|
| Time (Min) | (%) | (%) |
| 00:00 | 70 | 30 |
| 17:00 | 0 | 100 |
| 20:00 | 70 | 30 |
| 23:00 | 70 | 30 |

Method B: Detector: 220 nm

| | 0.1% TFA in Water | 0.1% TFA in CH₃CN |
|---|---|---|
| Time (Min) | (%) | (%) |
| 00:00 | 90 | 10 |
| 18:00 | 0 | 100 |
| 19:00 | 90 | 10 |
| 20:00 | 90 | 10 |

In the alternative, HPLC analyses were performed using methodology with modification to adjust the method to available column dimensions and to alter the gradient five minutes after the last observed impurity. HPLC conditions for the analyses are provided below.

Column: Synergi 4 μm polar-RP 80A 4.6×250 mm

Column Temperature: 40° C.

Detection: 210 nm (PDA)

Flow Rate: 1.0 mL/min

Injection Volume: 10 μL (microliters)

Mobile Phase: A: 0.1% TFA in water

B: 0.1% TFA in Methanol

Gradient:

| Time (min) | % A | % B |
|---|---|---|
| 0 | 50% | 50% |
| 20:00 | 30% | 70% |
| 25:00 | 0% | 100% |
| 42:00 | 0% | 100% |
| 44:00 | 50% | 50% |
| 60:00 | 50% | 50% |

Run Time: 60 minutes

Sample Concentration: approximately 1 mg/mL

Sample Diluent: Methanol

X-Ray Powder Diffraction analysis (XRPD) was at times also performed by placing sufficient sample onto zero background Si plates and acquiring a diffraction pattern using the following conditions:

X-ray Tube: Cu K., 40 kV, and 40 mA

Slits:
Divergence Slit: 1.00 deg
Scatter Slit: 1.00 deg
Receiving Slit: 0.30 mm

Scanning:
Scan Range: 3.0-45.0 deg
Scan Mode: Continuous
Scan Speed: 2.0000 deg/min
Sampling Pitch: 0.04 deg
Preset Time: 1.20 seconds

Dynamic vapor sorption analysis (DVS) was at times performed on the sample “as is”. Samples were placed in sample pan and underwent a 2-hour drying in a dry nitrogen stream. The samples were then analyzed under the following conditions:

Temperature: 25° C.

Isothermal Sequence:
Adsorption: 10-90% RH; step size 10%
Desorption: 85-0% RH; step size 10%

Each RH point was obtained after the weight loss curve reached an asymptote. After the isotherm was complete, the sample was heated to 80° C. until the weight loss curve reached an asymptote or for 4 hours maximum.

Experimental Section

Reagents and solvents were used as received from commercial vendors without further purification. Proton and carbon nuclear magnetic resonance spectra were obtained on a Bruker AV-500 at 500 MHz for proton and 125 MHz for carbon. Spectra are given in ppm (δ) and coupling constants, J, are reported in Hertz. Tetramethylsilane was used as an internal standard for proton spectra and the solvent was used as the reference peak for carbon spectra. Thermal gravimetric analyses and Differential Scanning Calorimeter analyses were conducted using a Mettler Toledo (Columbus, Ohio) 851 TGA and 821e DSC Systems, respectively. Moisture sorption analyses were conducted using dynamic vapor sorption tests run by a Hiden Isochema (Warrington, UK) IGASorp System. X-ray powder diffraction analyses were conducting using a Shimadzu XRD-6000 System. High performance liquid chromatograpy analyses were conducted using a Waters 2690 HPLC System. Optical rotation data was not obtained since there are no stereogenic centers present in DA-5018 or intermediates prepared in the synthesis.

### EXAMPLE 1

### Preparation of 3-(3,4-Dimethylphenyl)acrylonitrile (2)

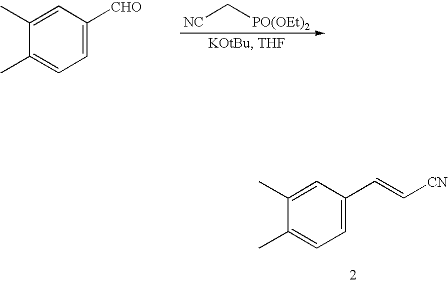

A 12-L, round-bottom flask was charged with a solution of diethyl (cyanomethyl)phosphonate (600 g, 3.8 mol, 1.2 eq) in dry THF (6 L). After the solution had been cooled in an ice bath (internal temperature 18° C.), potassium t-butoxide (380 g, 3.4 mol, 1.2 eq) was added to the solution portionwise over 30 min. The rate of addition was dictated by the temperature of the reaction solution, such that the temperature was between 14 and 20° C. When the addition was complete, the reaction mixture was stirred for an additional 1.5 h. A solution of 3,4-dimethylbenzaldehyde (379 g, 2.8 mol, 1.0 eq) in dry THF (1.6 L) was then added dropwise. After 30 min, the reaction mixture had become a viscous gel. Dilution of the reaction mixture with dry THF (1.5 L) facilitated stirring and addition of the aldehyde solution was continued. After stirring for another hour after the aldehyde addition was complete, HPLC analysis of the reaction mixture indicated the reaction was complete. The two peaks observed in the HPLC trace corresponded to the cis- and trans- isomers of the acrylonitrile product. The precipitated solids in the reaction mixture were completely dissolved following the addition of water (3 L). The product was extracted with isopropyl acetate (2×4 L). The organic extracts were dried (Na₂SO₄), clarified, and the filtrate was concentrated to afford a slightly red crystalline solid. Residual solvent was removed by placing the solid in a vacuum oven (45° C.) to give 456 g (quantitative) of 3-(3,4-Dimethylphenyl)acrylonitrile (2); HPLC 87.0% trans-, 13.0% cis-(AUC). The ¹H NMR spectrum was consistent with the assigned structure.

### EXAMPLE 2

### 3-(3,4-Dimethylphenyl)propylamine (3)

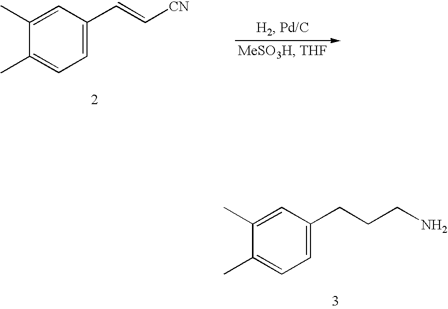

A 20-L, 316 stainless steel autoclave was charged with 5% palladium-on-carbon (543 g, 50% wet, Johnson-Matthey type A405023-5) followed by a solution of 3-(3,4-dimethylphenyl)acrylonitrile 2 [1087 g, 6.7 mol] in dry THF (10.8 L) and methanesulfonic acid (806 mL, 10.7 mol). The atmosphere in the reaction vessel was evacuated and back-filled with nitrogen three times with stirring. This was repeated using hydrogen in place of nitrogen (no stirring). The reaction vessel was charged with 50 psi of hydrogen gas and stirred at 2000 rpm for 2 h. The reaction vessel was purged with nitrogen prior to the removal of an aliquot of the reaction mixture for HPLC analysis. When the reaction was determined to be complete, the reaction mixture was diluted with water (4 L), filtered, and washed with water (2 L). Dichloromethane (2 L) was added to the filtrate and the resultant two layers were separated. The aqueous layer was adjusted to pH 14 using 2 N sodium hydroxide (2 L) and washed with CH₂Cl₂ (2×4 L). The organic extracts were combined and evaporated to dryness to give 767 g of 3-(3,4-Dimethylphenyl)propylamine 3 as a pale green liquid (67%); HPLC 91.8% (AUC). The ¹H NMR spectrum was consistent with the assigned structure.

### EXAMPLE 3

### Preparation of Hydrochloride Salt of 3-(3,4-Dimethylphenyl) propylamine (3)

3-(3,4-Dimethylphenyl)propylamine 3 [50 g, 306 mmol] was dissolved in methanol (150 mL) and cooled to 0-5° C. Concentrated hydrochloric acid (38 mL, 459 mmol) was added dropwise maintaining an internal temperature of ≦5° C. and the resultant slurry was stirred for 3 h at 0-5° C. The cold slurry was filtered to give a white solid which was oven dried under reduced pressure at 50° C. to give 20.4 g of the hydrochloride salt of 3-(3,4-Dimethylphenyl)propylamine 3 [33% yield]; HPLC 95.6% (AUC). To obtain a second crop, the filtrate volume was reduced to one half its original volume under reduced pressure and the resultant slurry was cooled to 0-5° C. for 1 h. The cold slurry was filtered to give a white solid which was oven dried under reduced pressure at 50° C. to give an additional 19.5 g of the hydrochloride salt of 3-(3,4-Dimethylphenyl)propylamine 3 [32% yield); HPLC 96.8% (AUC). A total of 40 g of the hydrochloride salt was obtained in 65% combined yield.

### EXAMPLE 4

### Preparation of N-[3-(3,4-Dimethylphenyl)propyl]-2-(4-hydroxy-3-methoxyphenyl)-acetamide (4)

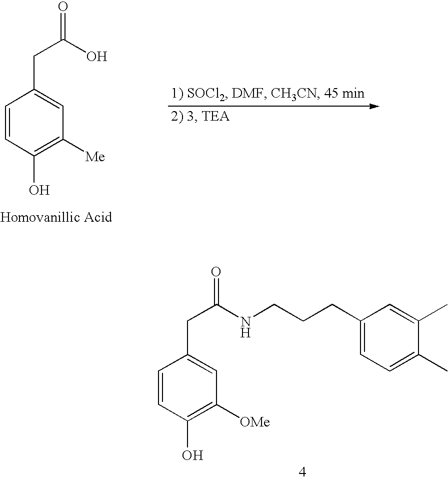

Hydrochloride salt of 3-(3,4-Dimethylphenyl) propylamine 3 [376.3 g, 1.89 mol] was stirred in a mixture of toluene (1.1 L) and 2 N sodium hydroxide (1.1 L) solution until total dissolution occurred. The two layers were separated and the aqueous layer was washed with toluene (1.1 L). The organic layers were combined and concentrated under reduced pressure to a volume of 1 L affording 3-(3,4-Dimethylphenyl)propylamine 3 as a dry toluene solution.

Homovanillic acid (379 g, 2.1 mol) was dissolved in acetonitrile (4.5 L) and dimethylformamide (0.5 mL). Thionyl chloride (151 mL, 2.1 mol) was added dropwise over 30 min and the reaction mixture was stirred for 2 h while cooled to 0-5° C. The dry toluene solution of amine 3 was diluted with triethylamine (664 mL, 4.7 mol) and added to the cooled reaction mixture over 3 h maintaining an internal temperature of ≦10° C. The reaction mixture was warmed to ambient temperature and water (750 mL) was added. The resultant solution was concentrated to a volume of approximately 3 L under reduced pressure and water (1 L) and isopropyl acetate (1 L) were added. The resultant two layers were separated and the organic layer was washed with water (1 L). To the organic layer was added 2 N sodium hydroxide solution (1 L) and the biphasic mixture was stirred for 1 h. The pH of the biphasic mixture was carefully adjusted from 14 to 8 with 6 M hydrochloric acid and the two layers were separated. The aqueous layer was washed with isopropyl acetate (1 L) and the organics were combined, washed with 10% w/v aqueous potassium bicarbonate solution (1 L), water (1 L) and evaporated to dryness to give 538 g of N-[3-(3,4-Dimethylphenyl)propyl]-2-(4-hydroxy-3-methoxyphenyl)-acetamide 4 as a dark gum [79% after correction for solvent content]; HPLC 83.5% (AUC).

### EXAMPLE 5

### Preparation of 2-[4-(Cyanomethoxy)-3-methoxyphenyl]-N-[(3,4-dimethylphenyl)propyl]acetamide (5)

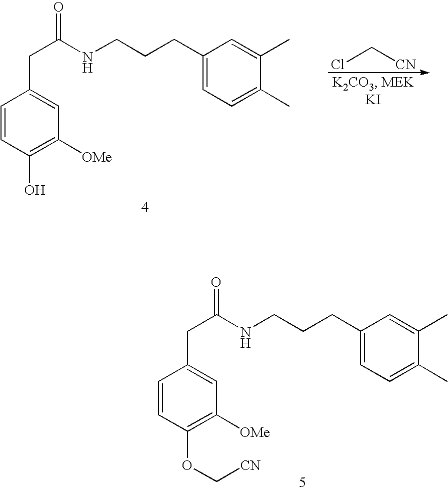

N-[3-(3,4-Dimethylphenyl)propyl]-2-(4-hydroxy-3-methoxyphenyl)-acetamide 4 [639 g, 1.95 mol] was dissolved in methyl ethyl ketone (9.5 L). Potassium carbonate (477.8 g, 4.88 mol, 325 mesh), chloroacetonitrile (187 mL, 2.93 mol) and potassium iodide (161 g, 1.0 mol) were added and the reaction mixture was heated to 75° C. for 24 h. The reaction mixture was cooled to room temperature, water (1.3 L) was added, and the mixture stirred until dissolution of the solids was complete. The resultant two layers were separated and the organic layer was washed with water (1.3 L). The combined aqueous extracts were washed with isopropyl acetate (IPAc) (1.3 L) and all of the organic extracts were combined and evaporated to dryness to give a brown solid. Methanol (2 L) was added to the brown solid and the resultant slurry was stirred at 0-5° C. for 3 h. The cold slurry was filtered, washed with cold methanol (MeOH) (1 L), and oven dried under reduced pressure at 50° C. to give 515.2 g of nitrile 5 as a beige solid [72%]; HPLC 97.4% (AUC). The ¹H NMR spectrum was consistent with the assigned structure.

### EXAMPLE 6

### Preparation of DA-5018 (1), 80 g Scale

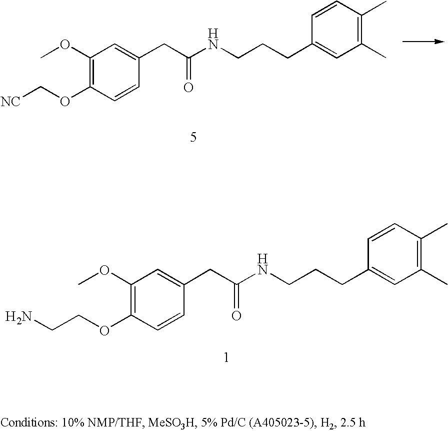

A 1.6-L pressure reactor was charged with 2-[4-(cyanomethoxy)-3-methoxyphenyl]-N-[(3,4-dimethylphenyl)-propyl]acetamide 5 [80.0 g, 228 mmol], a solution of 10% dry NMP in dry THF (1 L), and 5% palladium-on-carbon (Johnson-Matthey type A405023-5, 40 g, 50% wet). Methanesulfonic acid (23.7 mL, 365 mmol, 1.6 equiv) was then added and the temperature of the reaction mixture increased to 30° C. The reaction vessel was evacuated and back-filled with nitrogen three times with stirring. The process was repeated using hydrogen in place of nitrogen an additional three times. However, the mixture was not stirred during the hydrogen purges. The reaction vessel was then charged with 50 psi of hydrogen gas and stirred at 1200 rpm. The temperature of the reaction mixture increased from 25° C. to 40° C. over 3 min. There was no pressure increase as a result of the exotherm. After a total of 5 min had elapsed, the temperature began to decrease. After 2.5 h the temperature of the reaction mixture was 37° C. The reactor was then purged with nitrogen prior to the removal of an aliquot of the reaction mixture. HPLC analysis of an aliquot of the mixture verified the reaction was complete and DA-5018 was the major component in approximately 93% (AUC). The reaction mixture was diluted with water (1.2 L), filtered, and washed with an additional portion of water (2 L). The combined filtrate was extracted with isopropyl acetate (2×1.2 L). An emulsion formed during the second extraction which was difficult to break. The aqueous layer was then cooled by the addition of ice to the solution and then treated with 2 M NaOH (1.2 L). A white precipitate formed which was collected by vacuum filtration. The solid was dried in a vacuum oven at 40° C. for 48 h to afford DA-5018 1 as an off-white solid [68.7 g, 85%]; HPLC 98.4% purity (AUC). The ¹H NMR spectrum was consistent with the assigned structure. HPLC analysis of the isopropyl acetate extracts and the aqueous filtrate showed only trace amounts of DA-5018 remaining in solution.

### EXAMPLE 7

### Preparation of DA-5018 (1), 350 g Scale

A 20-L, 316 stainless steel autoclave was charged with 5 [350 g, 1.00 mol], a solution of 10% dry NMP in dry THF (4.45 L), 5% palladium-on-carbon (Johnson-Matthey type A405023-5, 175 g, 50% wet) and methanesulfonic acid (104 mL, 1.60 mol, 1.6 eq). The reaction mixture was cooled to 9.6° C. (chilled glycol circulating cooling coil inside reactor). The atmosphere in the reaction vessel was evacuated and back-filled with nitrogen three times with stirring. The process was repeated using hydrogen in place of nitrogen an additional three times. However, the mixture was not stirred during the hydrogen purges. The reaction vessel was then charged with 50 psi of hydrogen gas and stirred at 2000 rpm. The temperature of the reaction mixture increased from 10° C. to 14.4° C. over 10 min. There was no pressure increase as a result of the exotherm. After a total of 15 min had elapsed, the temperature began to decrease. The chiller supplying 0° C. coolant to the reactor was then turned off, allowing the reaction mixture to warm slowly to ambient temperature. After 2.5 h the temperature of the reaction mixture was 19° C. The reactor was then purged with nitrogen prior to the removal of an aliquot of the reaction mixture. HPLC analysis of an aliquot verified the reaction was complete and DA-5018 1 was the major component in approximately 98% conversion (AUC). The reaction mixture was diluted with water (5 L), filtered, and washed with an additional portion of water (2.5 L). A sample of the filter cake was washed with water and the washings were analyzed by HPLC, verifying no product remained on the cake. The combined filtrates were then extracted with isopropyl acetate (4.5 L). The aqueous layer was cooled by the addition of ice to the solution and then 2 M NaOH (1 L) was added dropwise with stirring over 30 min. A white precipitate formed which was collected by vacuum filtration. The solid was dried in a vacuum oven at 40° C. for 3 d to afford DA-5018 1 as an off-white solid [292 g, 82%]; HPLC 98.8% (AUC). The ¹H NMR spectrum was consistent with the assigned structure. HPLC analysis of the aqueous filtrate did show DA-5018 1 remaining in solution in addition to a significant amount of NMP.

### EXAMPLE 8

### Preparation of DA-5018 (1), 1 kg Scale

A 20-L, 316 stainless steel autoclave was charged with 2-[4-(cyanomethoxy)-3-methoxyphenyl]-N-[(3,4-dimethyl-phenyl)propyllacetamide 5 [960 g, 2.7 mol], a solution of 10% NMP in THF (12.5 L), 5% palladium-on-carbon (Johnson-Matthey type A405023-5, 433 g, 50% wet) and methanesulfonic acid (285 mL, 4.4 mol, 1.6 eq). The atmosphere in the reaction mixture was cooled to 5° C. (cooling coil inside reactor). The reaction vessel was evacuated and back-filled with nitrogen three times with stirring. The process was repeated using hydrogen in place of nitrogen an additional three times. However, the mixture was not stirred during the hydrogen purges. The reaction vessel was then charged with 50 psi of hydrogen gas and stirred at 2000 rpm. The temperature of the reaction mixture increased from 5° C. to 14° C. over 15 min. There was no pressure increase as a result of the exotherm. After a total of 15 min had elapsed, the temperature began to decrease. The chiller supplying 0° C. coolant to the reactor was then turned off, allowing the reaction mixture to warm slowly to ambient temperature. After 2.5 h the temperature of the reaction mixture was 25° C. An aliquot was removed from the reaction using the reactor's sampling tube. HPLC analysis indicated DA-5018 was present in 70% conversion (AUC) in addition to residual starting nitrile 5 and other impurities. The reactor was then purged with nitrogen so that it could be opened to remove a more representative aliquot of the reaction mixture. Upon opening the reactor, it was obvious that the stirring was not thorough and a substantial portion of the slurry was coating the sides of the reactor and not mixing well. Additional THF (2 L) was used to wash down the sides of the reactor and the apparatus was reassembled. After further exposure to H₂ at 50 psi for 1 h, the reaction mixture was diluted with water (10 L), filtered, and washed with additional water (4 L) to ensure no DA-5018 1 remained on the solids. HPLC analysis of the filtrate indicated DA-5018 1 was the major component (74%, AUC). However, phenol 4 was also present in 22% in addition to other minor impurities typically observed in this reaction. Phenol 4 was efficiently removed from the aqueous solution by extraction with isopropyl acetate (3×10 L). HPLC analysis of the aqueous solution following these extractions indicated the phenol content was <1% and the DA-5018 1 content has increased to 95%. The aqueous solution was then cooled in ice (<5° C.) and treated dropwise with aqueous 2 M NaOH (4 L), resulting in the precipitation of a tan solid. The solid was isolated by vacuum filtration. The filtration was very slow which hindered attempts to wash the solid with additional water. The solid was therefore suspended in water (10 L) and stirred vigorously for 30 min. HPLC analysis of this material indicated the purity of DA-5018 1 had been increased to 97% (AUC) through this process. The solid was isolated by vacuum filtration and triturated with acetonitrile to assist in the removal of residual water. After drying the solid to a constant weight in a vacuum oven (35° C.), DA-5018 1 was obtained as a tan solid [610 g, 64%]; HPLC 97.0% (AUC). The ¹H NMR spectrum was consistent with the assigned structure.

Alternate Synthesis for DA-5018 1—Variation

As set forth in the following stages for clarity, the present subject matter further contemplates other, equivalent schemes and variations thereof for preparing the instant compounds.

For example, variations in the type of reaction vessel (glass vessel or pressure vessel, e.g. steel), hydrogen pressure (1 atm to 3 atm), specificity of the Pd/C catalyst (specific 5% vs. non-specific 10%), solvent selection (methanol vs. MTBE), and order of substrate addition, are all contemplated as within the scope of the present processes and may be varied to affect purity, yield, and efficient use of available reagents.

One such example is provided below.

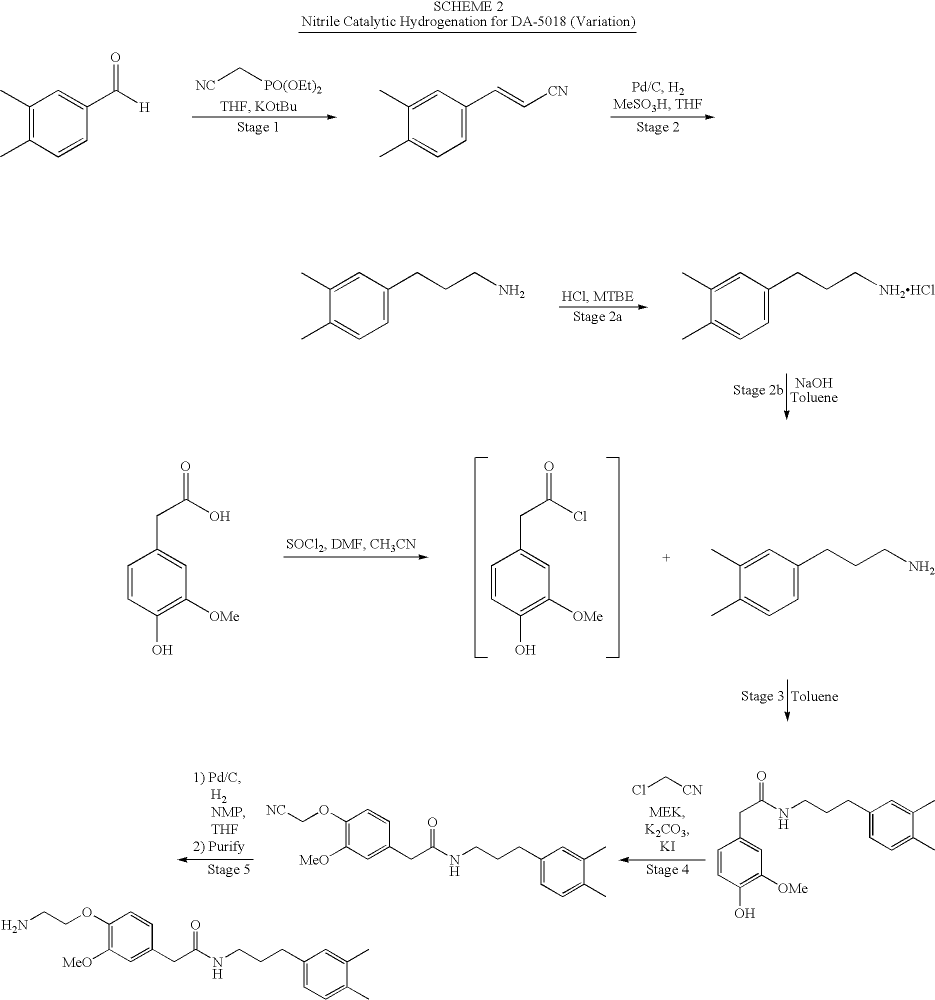

### EXAMPLE 9

### Preparation of 3-(3,4-dimethylphenyl)acrylonitrile (2)

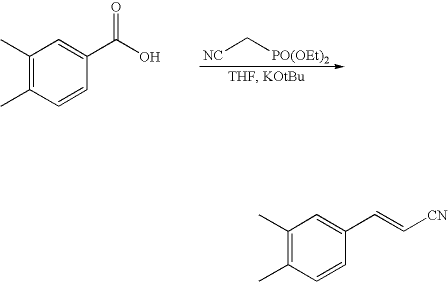

Potassium t-butoxide (0.55 Kg, 4.9 mol, 1.2 eq) was added portionwise to a solution of diethyl(cyanomethyl)-phosphonate (0.87 Kg, 4.9 mol, 1.2 eq) in tetrahydrofuran (THF, 10.0 L) at 15 to 20° C. (addition time: 5 to 15 minutes) and the resulting solution stirred at 5 to 20° C. for 90 minutes. To the resultant was added a solution of 3,4-dimethylbenzaldehyde (0.55 Kg, 4.01 mol, 1.0 eq) as a solution in THF (2.3 L) at 15 to 20° C. (addition time: 45 to 60 minutes) followed by a line rinse of THF (0.8 L). The reaction mixture was stirred for 1 hour at 15 to 20° C. after which time HPLC and/or ¹H NMR analysis indicated reaction completion. The reaction mixture was quenched with water (4.4 L), extracted with isopropyl acetate (2×5.8 L), the organic extracts combined, dried over sodium sulfate and concentrated under vacuum at 35 to 40° C. to afford 3-(3,4-dimethylphenyl) acrylonitrile 2 as a beige solid (0.66 Kg, 102%).

The process variation here is the use of 20 L glassware with a maximum input of 0.56 Kg of 3,4-dimethylbenzaldehyde.

### EXAMPLE 10

### Preparation of 3-(3,4-dimethylphenyl)propylamine (3)

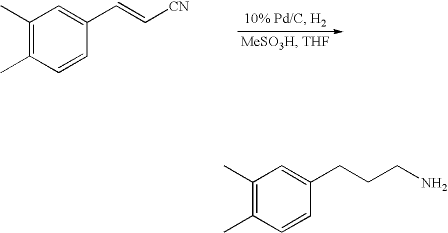

A solution of 3-(3,4-dimethylphenyl)acrylonitrile 2 (1.29 Kg, 8.2 mol, 1.0 eq) and methanesulfonic acid (0.96 L, 14.8 mol, 1.8 eq) in tetrahydrofuran (THF, 10.3 L) was added under nitrogen to 10% palladium on carbon (50% water wet, 0.32 Kg). Three vacuum-nitrogen purge cycles were followed by 3 vacuum-hydrogen purge cycles and an atmosphere of hydrogen introduced. The reaction mixture was stirred under hydrogen for 3 hours at 15 to 25° C. after which time HPLC and or ¹H NMR analysis indicated reaction completion. The reaction mixture was quenched with water (4.7 L), stirred for 5 to 10 minutes, filtered and the filtrates concentrated under vacuum at 35 to 40° C. until solvent collection ceased. The filter-cake was washed with water (6.5 L) and the aqueous filtrate combined with the aqueous concentrate. The combined aqueous solution was washed with MTBE (6.5 L), the pH adjusted to pH 14 with aqueous sodium hydroxide solution (6 M, 3.0 L), the resultant extracted with MTBE (2×5.2 L), the extracts combined and concentrated under vacuum at 35 to 40° C. to afford 3-(3,4-dimethylphenyl)propylamine 3 as a yellow oil (0.76 Kg, 70.4%).

The process variation here is the use of atmospheric pressure of hydrogen (16 psi), the use of a non-specific 10% Pd/C (50% wet paste) catalyst, the reduction of catalyst loading from 0.5% w/w to 0.25% w/w with respect to 3-(3,4-dimethylphenyl)acrylonitrile charge, the use of rotary evaporation to remove the THF, extraction with MTBE instead of dichloromethane, and the use of 20 L glassware. This illustrates the robust nature of the broadly defined process.

### EXAMPLE 10a

### Preparation of 3-(3,4-dimethylphenyl)propylamine HCl

Concentrated hydrochloric acid (0.86 L, 10.33 mol, 1.5 eq) was added to a solution of 3-(3,4-dimethylphenyl)propylamine 3 (1.13 Kg, 6.89 mol, 1.0 eq) in MTBE (11.3 L) at 0 to 5° C. (addition time: 75 to 90 minutes). The resulting slurry was stirred for 3 h at 0 to 5°0 C., filtered, the filter-cake washed with MTBE (2.2 L) and the collected solids dried under vacuum at 45 to 50° C. for 16 h to afford 3-(3,4-dimethylphenyl)propylamine hydrochloride as an off-white solid (1.05 Kg, 76.0%).

The process variation here includes the formation of the hydrochloride salt in MTBE instead of methanol and the continued use of the glass reaction vessel.

### EXAMPLE 10b

### Preparation of 3-(3,4-dimethylphenyl)propylamine (3)

Aqueous sodium hydroxide solution (2.0 M, 5.7 L) was added to a suspension of 3-(3,4-dimethylphenyl)propylamine hydrochloride (1.90 Kg, 9.54 mol, 1.0 eq) in toluene (5.7 L) at 0 to 10° C. (addition time: 15 to 30 minutes). The resulting mixture was stirred for 15 to 20 minutes at 0 to 10° C. The layers were separated, and the aqueous phase extracted with toluene (5.7 L). The organic extracts were combined and concentrated under vacuum at 35 to 40° C. to afford 3-(3,4-dimethylphenyl)propylamine 3 as a red-brown oil (1.43 Kg, 92.1%).

### EXAMPLE 11

### Preparation of N-[3-(3,4-dimethylphenyl)propyl]-2-(4-hydroxy-3-methoxyphenyl)acetamide (4)

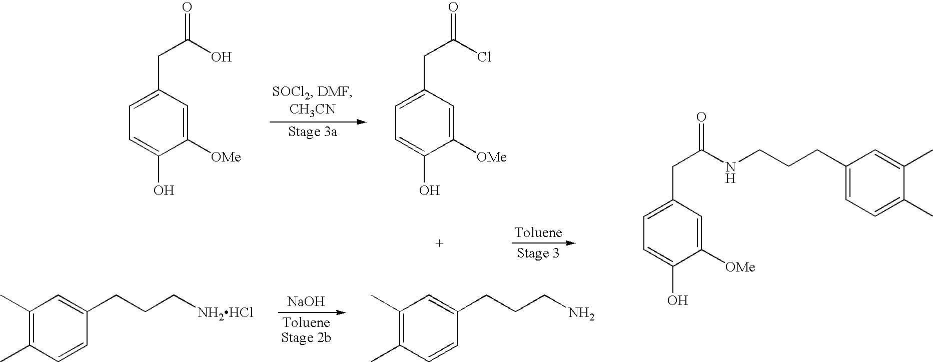

Thionyl chloride (0.36 L, 4.87 mol. 1.11 eq) was added to a suspension of homovanillic acid (0.89 Kg, 4.87 mol, 1.11 eq) and N,N-dimethylformamide (1.1 mL) in acetonitrile (10.5 L) at 10 to 20° C. (addition time: 15 to 25 minutes). The resulting hazy solution was cooled to and stirred at 0 to 5° C. for 1.5 to 2.5 h, the resulting solution added to a solution of 3-(3,4-dimethylphenyl)propylamine 3 (0.72 Kg, 4.39 mol. 1.0 eq) and triethylamine (1.54 L, 11.06 mol, 2.52 eq) in toluene (1.4 L) at 0 to 10° C. (addition time: 2.5 to 3 h) and the reaction mixture stirred to 15 to 20° C. overnight. HPLC analysis indicated reaction completion. The mixture was quenched with water (1.7 L) at 10 to 20° C. (addition time: 10 to 15 minutes) to give a dark brown solution which was concentrated under vacuum at 35 to 40° C. to approximately 10 volumes with respect to the 3-(3,4-dimethylphenyl)propylamine 3 charge. To the resultant were added water (2.3 L) and isopropyl acetate (2.3 L), the layers separated and the upper organic layer washed with water (2.3 L). The organics were treated with aqueous sodium hydroxide solution (2.0 M, 2.3 L), the resulting biphasic mixture stirred for 60 to 75 minutes at 15 to 20° C., the pH adjusted to a pH of 7.5 to 8.5 by the addition of hydrochloric acid (6 M, 0.5 L) and the layers separated. The lower aqueous phase was extracted with isopropyl acetate (2.3 L), the organics combined, washed with aqueous sodium bicarbonate solution (10% w/v, 2.3 L) and water (2.3 L) and concentrated under vacuum at 35 to 40° C. to afford N-[3-(3,4-dimethylphenyl)propyl)-2-(4-hydroxy-3-methoxyphenyl) acetamide 4 as a dark oil (1.25 Kg, 86.6%).

The process variation here is that the addition of the substrates is reversed, i.e. the in-situ generated acid chloride was added to the solution of the 3-(3,4-dimethylphenyl)propylamine, and the continued use of the glass reaction vessel.

### EXAMPLE 12

### Preparation of crude 2-[4-(cyanomethoxy)-3-methoxyphenyl]-N-[(3,4-dimethylphenyl)propyl]acetamide (5)

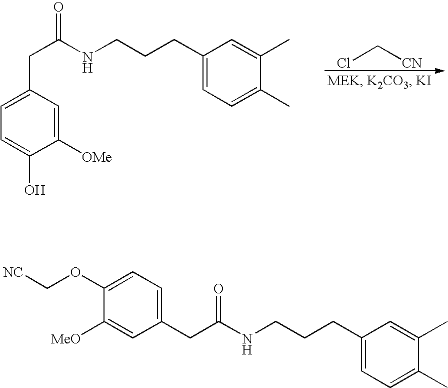

Chloroacetonitrile (0.29 Kg, 3.83 mol, 1.5 eq), potassium carbonate (0.70 Kg, 5.1 mol, 2.0 eq) and potassium iodide (0.21 Kg, 1.27 mol, 0.5 eq) were added in one portion to a solution of N-[3-(3,4-dimethylphenyl)propyl]-2-(4-hydroxy-3-methoxyphenyl)acetamide 4 (0.83 Kg, 2.55 mol, 1.0 eq) in MEK (12.4 L). The reaction mixture was heated to and maintained at 73 to 77° C. with efficient stirring for 20 to 26 h after which time HPLC analysis indicated reaction completion. The resultant was cooled to 15 to 20° C., quenched with water (1.7 L) at 15 to 20° C. and stirred in this temperature range until full dissolution was obtained (10 to 20 minutes). The layers were separated, the organic phase washed with water (1.7 L), the combined aqueous washes extracted with isopropyl acetate (1.7 L, overnight separation) and the combined organic extracts concentrated under vacuum at 35 to 40° C. to give a dark, amorphous solid (1.39 Kg, 150%).

### EXAMPLE 13a

### Purification of 2-[4-(cyanomethoxy)-3-methoxyphenyl]-N-[(3,4-dimethylphenyl)propyl]acetamide (5)

Methanol (7.8 L) was charged to crude 2-[4-(cyanomethoxy)-3-methoxyphenyl]-N-[(3,4-dimethylphenyl)propyl]acetamide 5 (4.39 Kg) and the resulting slurry cooled to and aged at 0 to 5° C. for 2 to 2.25 h. The mixture was filtered, the filter-cake washed with cold (0 to 5° C.) methanol (4.0 L) and dried under vacuum at 35 to 40° C. (63 h) to give 2-(4-(cyanomethoxy)-3-methoxyphenyl]-N-[(3,4-dimethylphenyl)propyl]acetamide 5 as a beige solid (1.85 Kg, 66.7% recovery).

### EXAMPLE 13b

### Re-work of 2-[4-(cyanomethoxy)-3-methoxyphenyl]-N-[(3,4-dimethylphenyl)propyl]acetamide (5) to Remove Residual Iodide

Water (8.7 L) was added to 2-[4-(cyanomethoxy)-3-methoxyphenyl)-N-[(3,4-dimethylphenyl)propyllacetamide 5 (1.75 Kg) and the resulting mixture efficiently stirred at 15 to 20° C. for 2 to 2.5 h. The resultant was filtered, the filter-cake washed with water (3.5 L) and the collected solids dried under vacuum at 45 to 50° C. for 36 h to obtain a 96% recovery, 1.68 Kg, after correction for water.

### EXAMPLE 14

### Preparation of crude DA-5018 (1)

Methanesulfonic acid (0.25 L, 3.79 mol, 1.67 eq) was added to a solution of 2-[4-(cyanomethoxy)-3-methoxyphenyll-N-[(3,4-dimethylphenyl)propyl]acetamide 5 (0.83 Kg, 2.27 mol, 1.0 eq) in tetrahydrofuran (9.35 L) and 1-methyl-2-pyrrolidinone (1.04 L). 10% Palladium on carbon (50% water wet, 0.42 Kg) was then charged, 3 vacuum-nitrogen purge cycles and 3 vacuum-hydrogen purge cycles completed and one atmosphere of hydrogen gas was introduced. Stirring was maintained at 450 to 550 rpm and 8 to 20° C. for 2 h after which time reaction completion was confirmed by HPLC analysis. The reaction mixture was quenched with water (12.5 L) at 8 to 25° C., stirred for 5 to 10 minutes and filtered under nitrogen. The filter-cake was washed with water (20.8 L) and the combined filtrates washed with isopropyl acetate (2×12.5 L). The aqueous layer was cooled to 0 to 10° C., treated with aqueous sodium hydroxide solution (2.0 M, 12.5 L), the resulting suspension aged at 0 to 10° C. for 30 to 45 minutes, filtered and the filter-cake washed with water (1.5 L). The crude DA-5018 1 was dried under vacuum at 60 to 65° C. for up to 68 h.

The process variation here includes the use of atmospheric pressure of hydrogen (about 16 psi), the use of a non-specific 10% Pd/C (50% water wet) catalyst, and the use of a 20 L glass reaction vessel.

III. Convergent Synthesis

In another preferred embodiment, the present subject matter pertains broadly to processes for deprotecting an intermediate compound to produce an amine compound.

Convergent Route 1—Deprotection

In a particularly preferred embodiment, a process is provided herein for preparation of an amine product, which comprises deprotecting an intermediate compound of Formula II to obtain the corresponding amine. Formula II represents a genus of compounds which are deprotected:

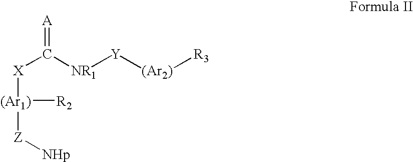

wherein

X is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR_{4,} or SR₅;

Y is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

Z is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

A is oxygen or sulfur wherein the sulfur is optionally substituted with 2 or 4 hydrogen, oxy, alkyl, alkyloxy, or alkylamino radicals;

R₁ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

Ar₁ is a heterocycle, aryl, or heteroaryl radical wherein Ar₁ is substituted in one to five places with R₂;

R₂ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

Ar₂ is a heterocycle, aryl, or heteroaryl radical wherein Ar₂ is substituted in one to five places with R₃;

R₃ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;

R₄ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo;

R₅ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo; and

p is a protecting group;

wherein said heterocycle is a radical of a monocyclic or bicyclic saturated heterocyclic ring system having 5-8 ring members per ring, wherein 1-3 ring members are oxygen, sulfur or nitrogen heteroatoms, which is optionally partially unsaturated or benzo-fused and optionally substituted by 1-2 oxo or thioxo radicals; said aryl is a phenyl or naphthyl radical; and said heteroaryl is a radical of a monocyclic or bicyclic aromatic heterocyclic ring system having 5-6 ring members per ring, wherein 1-3 ring members are oxygen, sulfur or nitrogen heteroatoms, which is optionally benzo-fused or saturated C₃-C₄-carbocyclic-fused.

Exemplary intermediate compounds of formula II which produce a preferred subgenus of final capsaicinoid amines, include those wherein:

X is a C₁₋₁₀ alkyl or C₂₋₁₀ alkenylene radical;

Y is a C₁₋₂₀ alkyl or C₂₋₁₀ alkenylene radical;

Z is a C₁₋₂₀ alkyl, C₁₋₂₀ alkyloxy, C₂₋₂₀ alkenylene, or C₂₋₂₀ alkenyleneoxy radical;

A is oxygen or sulfur;

R₁ is hydrogen, C₁₋₂₀ alkyl, or C₂₋₂₀ alkenylene;

Ar₁ is a C₃₋₂₀ carbocyclic ring or C₃₋₂₀ hetercyclic ring having one or more heteroatoms selected from O, N, or S, wherein Ar₁ is substituted in one to five places with R₂;

R₂ is hydrogen, C₁₋₂₀ alkyl, C₂₋₂₀ alkenylene, C₁₋₂₀ alkyloxy, C₂₋₂₀ alkenyleneoxy, C₁₋₂₀ thioalkyl, or C₂₋₂₀ thioalkenylene;

Ar₂ is a C₃₋₂₀ carbocyclic ring or C₃₋₂₀ hetercyclic ring having one or more heteroatoms selected from O, N, or S, wherein Ar₂ is substituted in one to five places with R₃;

R₃ is hydrogen, C₁₋₂₀ alkyl, C₂₋₂₀ alkenylene, C₁₋₂₀ alkyloxy, C₂₋₂₀ alkenyleneoxy, C₁₋₂₀ thioalkyl, or C₂₋₂₀ thioalkenylene; and

p is tert-butyloxycarbonyl (t-Boc).

In a preferred embodiment, the present processes relate to a process for preparation of DA-5018, which comprises:

1) deprotecting an intermediate compound of Formula III: 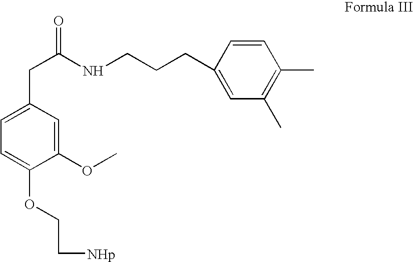
wherein p is a protecting group; and

2) obtaining DA-5018, especially DA-5018 having a high purity.

In a preferred embodiment, the DA-5018 produced according to this process is at least about 85% pure. In a particularly preferred embodiment, the DA-5018 produced according to this process is at least about 90% pure. In a most preferred embodiment, the DA-5018 produced according to this process is at least about 95% pure.

Novel Intermediates

Additional novel intermediates which are useful in the instant processes for constructing the larger protected amines, especially capsaicinoids, are further contemplated herein.

Examples include a compound of Formula IV:

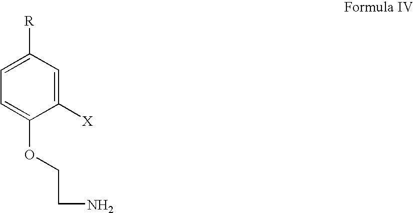

wherein

R is C₁₋₆ alkyl or C₂₋₆ alkenyl substituted with COOH or CONH₂; and

X is C₁₋₁₀ alkoxy, C₂₋₁₀ alkenoyl, or C₂₋₁₀ alkenoxy.

Novel intermediate compounds of Formula IV useful in the manufacture of capsaicinoids are considered among the preferred aspects herein, provided that R is not C₁—COOH, when X=methoxy (J. Med. Chem., Vol. 39, (1996) pp. 29-39), as shown below:

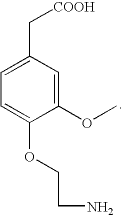

Novel protected intermediates useful in the manufacture of capsaicinoids can also include:

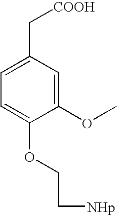

wherein p is a protecting group, preferably t-Boc.

Novel amido intermediates useful in the manufacture of capsaicinoids can include:

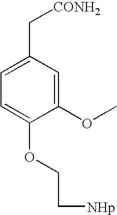

wherein p is a protecting group, preferably t-Boc.

Also considered within the preferred aspects is the protected intermediate useful in the manufacture of capsaicinoids:

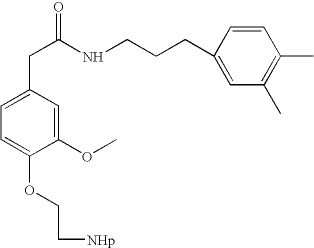

wherein p is a protecting group, preferably t-Boc.

Protecting Groups

The selection of a suitable protecting group depends upon the functional group being protected, the conditions to which the protecting group is being exposed and to other functional groups which may be present in the molecule. Suitable protecting groups are described in Greene and Wuts, “Protective Groups in Organic Synthesis”, John Wiley & Sons (1991), the entire contents of which are hereby incorporated by reference. The skilled artisan can select, using no more than routine experimentation, suitable protecting groups for use in the disclosed synthesis, including protecting groups other than those described below, as well as conditions for applying and removing the protecting groups.

Examples of suitable amino protecting groups include, without limitation, benzyloxycarbonyl, tert-butoxycarbonyl (t-Boc), and benzyl. In a preferred embodiment, tert-butoxycarbonyl (t-Boc) is the amine protecting group.

The protecting group t-Boc is particularly preferred in these intermediates since it provides crystallinity at the end of the process and yields a readily crystallizable intermediate.

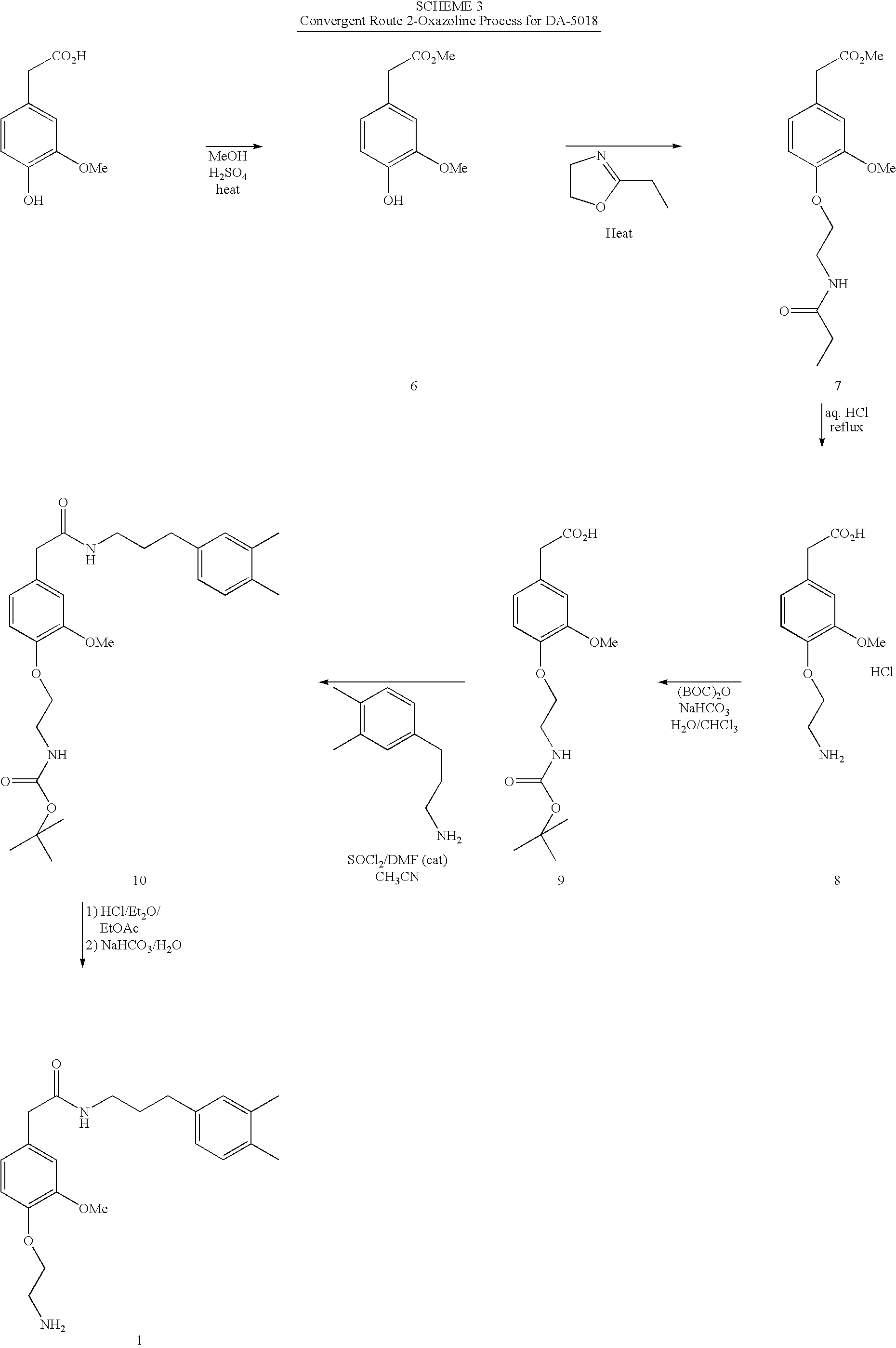

### EXAMPLE 15

### Methyl homovanillate (6)

To a stirred solution of homovanillic acid (100 g, 0.55 mol) in dry methanol (1.0 L) was added trimethyl orthoformate (25 mL) and concentrated sulfuric acid (5 mL) The solution was heated at reflux for 18 hours, at which point the reaction was complete by TLC analysis. Following cooling, the reaction mixture was concentrated under reduced pressure. The residue was dissolved in toluene (1.0 L) and this solution was washed with water (2×200 mL), saturated aqueous sodium bicarbonate solution (250 mL), saturated aqueous sodium chloride solution and dried over magnesium sulfate. After clarification, the filtrate was concentrated to dryness under reduced pressure and the residue was distilled (120-125° C./0.5 mm Hg vacuum) to afford 92 g of 6 in 85% yield, as a colorless oil. The NMR was consistent with the structure.

### EXAMPLE 16

### Methyl 4-(2-Propiamidoethoxy)-3-methoxyphenylacetate (7)

A nitrogen-inerted, stirred mixture of 6 (50 g, 0.25 mol) in 2-ethyloxazoline (500 mL) was heated to 160° C. for 8 hours. After cooling to room temperature, the reaction mixture was concentrated to dryness under reduced pressure. The residue was dissolved in toluene (750 mL) and washed with water (250 mL), saturated aqueous sodium bicarbonate solution (250 mL) and saturated aqueous sodium chloride solution (250 mL). The toluene solution was treated with a mixture of magnesium sulfate (5 g), decolorizing carbon (5 g), activated bentonite clay (10 g), and stirred for 3 hours. Following clarification, the toluene solution was passed through a silica gel pad (200 g) and the pad was rinsed with toluene (250 mL). The filtrate was concentrated to dryness under reduced pressure to afford a brown solid which was recrystallized from a methanol/water mixture to afford 45.7 g of 7 in 62% yield, as a light tan solid. The NMR was consistent with the structure. Literature references demonstrating this type of reaction include Morgan, T. K., Jr. et al., J. Med. Chem., Vol. 33 (1990) pp. 1087-1090; Lis, R. et al. J. Med. Chem., Vol. 33 (1990) pp. 2883-2891, the contents of which are hereby incorporated by reference in their entirety.

### EXAMPLE 17

### 4-(2-aminoethoxy)-3-methoxyphenylacetic acid hydrochloride (8)

A stirred suspension of 7 (40 g, 0.136 mol) in 6 N hydrochloric acid was heated to reflux for 20 hours, by which time the reaction mixture was homogenous. The reaction mixture was cooled to room temperature and then concentrated under reduced pressure, in a 60° C. water bath. The gummy residue was dissolved in warm isopropanol and then cooled overnight at 5° C. The solids were collected by filtration and vacuum dried at 60° C. for 48 hours to afford 27.1 g of 8, in 76% yield, as an off-white solid.

### EXAMPLE 18

### 4-(2-t-Butoxycarbonylaminoethoxy)-3-methoxyphenylacetic acid (9)

To a biphasic mixture of saturated aqueous sodium bicarbonate solution (100 mL) and chloroform (250 mL) was slowly added 8 (25 g, 95 mmol). The mixture was stirred for 30 min, then di-tert-butyl dicarbonate (25.9 g, 119 mmol, 1.25 eq) was added and the reaction mixture was warmed to 45° C. for 7 hours. The stirred mixture was cooled in an ice bath and the pH of the aqueous layer was adjusted to ˜4 by the slow addition of 1 N hydrochloric acid, and the biphasic mixture was vigorously stirred for 1 h. The layers were separated and the pH of the aqueous layer was again brought to ˜4 with 1 N HC_{1.} The aqueous layer was extracted with chloroform (5×50 mL). The combined chloroform extracts were dried over magnesium sulfate, filtered and evaporated to dryness to afford 29.5 g of 9, in 96% yield, as an off white solid.

### EXAMPLE 19

### N-[3-(3,4-dimethylphenyl)propyl]-2-(4-(2-t-butoxycarbonylaminoethyl)-3-methoxy)phenylacetamide (10)

To a solution of 9 (25 g, 77 mmol) in dry methanol (150 mL) was added 25% sodium methoxide in methanol solution (16.6 mL, 4.15 g of methoxide, 77 mmol) and the mixture was stirred for one hour at room temperature. The solution was concentrated to dryness under reduced pressure and the residue was dissolved in dry acetonitrile. This solution was concentrated to dryness under reduced pressure. This drying procedure was repeated two more times and then the residue was dissolved in dry acetonitrile (300 mL) and dry DMF (0.5 mL) was added. The solution was cooled to 0-5° C. and thionyl chloride (9.2 g, 77 mmol, 6.7 mL) was added dropwise over 30 minutes and the resulting white slurry was stirred for an additional 2 h at 0-5° C. In a separate flask, 3,4-dimethylphenylpropylamine 3 (12.6 g, 77 mmol) was dissolved in dry toluene (100 mL) and triethylamine (21.5 mL, 15.6 g, 2 eq) was added. This solution was added dropwise to the acid chloride slurry over 3 h, while maintaining the internal reaction temperature ≦10° C. The reaction mixture was warmed to ambient temperature and water (200 mL) was added. The resulting biphasic mixture was concentrated to approximately 300 mL under reduced pressure and water (250 mL) and isopropyl acetate (250 mL) were added. After mixing thoroughly, the layers were separated and the organic layer was washed with water (2×200 mL), saturated aqueous sodium bicarbonate solution (250 mL), saturated aqueous sodium chloride solution and dried over magnesium sulfate. The filtrate was concentrated to dryness under reduced pressure to afford a pale yellow solid (29.5 g). This solid was dissolved in a warmed mixture of cyclohexane (75 mL) and ethyl acetate (7.5 mL) and allowed to cool in an ice-water bath affording a white solid product. The solids were collected by filtration and the mother/wash liquors were concentrated to about 30 mL and a second crop was collected. The combined solids were vacuum dried to afford 28.9 g of 6, as a white solid, in 80% yield. The NMR was consistent with the structure.

### EXAMPLE 20

### Preparation of DA-5018 (1)

To a solution of 10 (20 g, 42.5 mmol) in ethyl acetate (500 mL) was dropwise added commercial 1 M HCl in diethyl ether solution (100 mL, 100 mmol, 2.4 eq). The mixture was stirred overnight at room temperature, by which time a white solid DA-5018 hydrochloride had crystallized. The solids were collected by filtration, washed with fresh ether and air dried to a constant weight to afford 13.3 g of DA-5018 hydrochloride. A mixture of 10 g of DA-5018 hydrochloride (24.6 mmol) in water (100 mL) was stirred until all the solids had dissolved. This solution was then cooled in a cold water bath and a solution of saturated aqueous sodium bicarbonate solution was added dropwise until the pH of the resulting stirred slurry was about 7. The slurry was stirred for 1 h and the solids were collected by filtration, washed with water and dried at 45° C., under reduced pressure, affording 8.3 g of DA-5018 1. The NMR was consistent with the spectra of DA-5018 1.

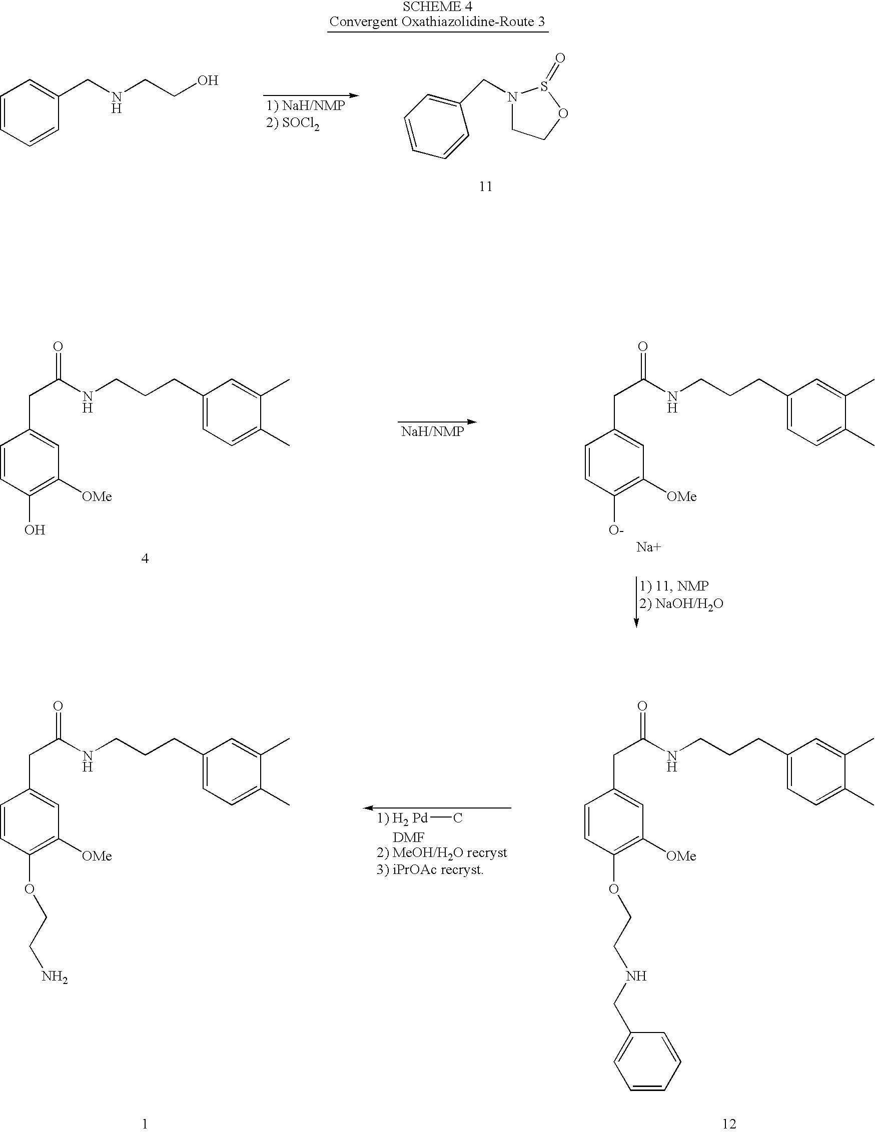

### EXAMPLE 21

### Preparation of 2-oxo-3-phenylmethyl-1,2,3-oxathiazolidine (11)

The title compound was prepared according to the procedure of Barker, et al. OPR&D, Vol. 3 (1999) p. 253, the entire contents of which are hereby incorporated by reference, utilizing N-benzylethanolamine (23.1 g, 21.7 mL, 153 nmol), sodium hydride (6.2 g of a 60% suspension in mineral oil, 153 mmol) and thionyl chloride (6.6 mL, 9.1 g, 76.5 mmol) in dry NMP (60 mL). This product was not isolated, but used directly in the next step.

### EXAMPLE 22

### Preparation of 4[2-(Phenylmethylamino)ethoxy)-3-methoxyphenyl]-N-[(3,4-dimethylphenyl)propyl]acetamide (12)

To a stirred, cooled suspension of sodium hydride (3.1 g of a 60% suspension in mineral oil, 76.5 mmol) in dry NMP (25 mL) was dropwise added a solution of 4 (25 g, 76.5 mmol) in dry NMP (25 mL). This solution was stirred at 10° C. for about 2 hours, until the hydrogen evolution ceased. The NMP solution of 11 was added over 15 min. The reaction mixture was heated to 70° C. for 18 h. After cooling, the reaction was quenched into an aqueous sodium hydroxide solution (6.2 g NaOH in 250 mL H₂O) and the resulting slurry was stirred for 2 h. The solids were collected by filtration, washed with water and vacuum dried at 60° C. to afford 25.3 g of crude 12. The dried product was suspended in a 10:1 mixture of cyclohexane and ethyl acetate (400 mL) and heated until the solids dissolved. Decolorizing carbon was added, stirred for 30 minutes and the warm mixture clarified. The filtrate was cooled in an ice bath for 2 hours and the resulting solid was collected by filtration. The solid was vacuum dried at 60° C. to afford 18.3 g of 12, in 52% yield, as an off white solid. The NMR was consistent with the structure.

### EXAMPLE 23

### Preparation of crude DA-5018 (1)

To a solution of 12 (10 g, 21.7 mmol) in DMF (150 mL) was added 10% palladium-on-carbon (1 g). The mixture was hydrogenated at 50 psi for 24 h and the solution was clarified through a pad of celite. The hydrogenation bottle and filter cake were washed with DMF (2×25 mL) and the combined filtrate was slowly added to stirred cold water (1 L). The resulting solids were collected by filtration and vacuum dried at 60° C. to afford 7.3 g of crude DA-5018 1 in 92% yield as a tan solid. The NMR was consistent with the NMR spectra of previous samples of crude DA-5018 1.

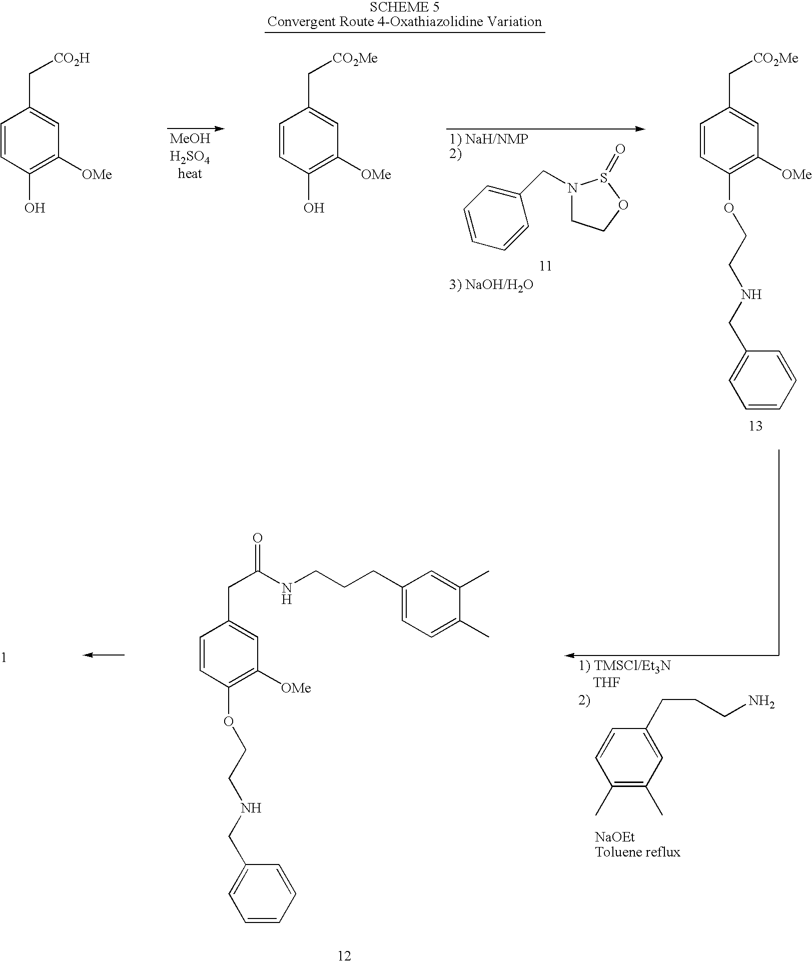

### EXAMPLE 24

### Preparation of Methyl 4-[2-((phenylmethylamine)ethoxy)-3-methoxy]phenylacetate (13)

To a stirred, cooled suspension of sodium hydride (3.1 g of a 60% suspension in mineral oil, 76.5 mmol) in dry NMP (25 mL) was dropwise added a solution of methyl homovanillate 6 (15 g, 76.5 mmol) in dry NMP (25 mL). The reaction mixture was stirred at 10° C. for 2 h until the hydrogen evolution ceased. A solution of 11 (15.1 g, 76.5 mmol) in dry NMP (75 mL) was added over 15 minutes. The reaction was heated to 70° C. for 24 h, and then cooled to room temperature. The reaction mixture was quenched into aqueous sodium hydroxide (6.2 g NaOH in 250 mL water) and the resulting slurry was stirred for 4 h. The solids were collected by filtration, washed with water (2×50 mL) and vacuum dried at 70° C. to afford 18.9 g of crude 13. The dried solid was dissolved in warm ethyl acetate (75 mL) and this solution was diluted with cyclohexane (300 mL) to the haze point. This suspension was cooled, with stirring, in an ice bath for 3 h and the solid was collected by filtration, dried under reduced pressure at 60° C. to afford 14.1 g of purified 13, in 56% yield, as a tan solid. The NMR spectrum was consistent with the structure.

### EXAMPLE 25

### Preparation of 4[2-(Phenylmethylamino)ethoxy)-3-methoxyphenyl]-N-[(3,4-dimethylphenyl)propyl]acetamide (12) (alternate procedure to Example 22)

To a stirred solution of 13 (14.1 g, 42.8 mmol) in dry THF (100 mL) was added triethylamine (5.85 g, 8.1 mL, 57.8 mmol). The solution was cooled to 0° C. and chlorotrimethylsilane (5.81 g, 6.8 mL, 53.5 mmol) was added dropwise over 15 minutes. The resulting solution was warmed to room temperature and stirred for 18 h. The resulting white slurry was clarified by vacuum filtration and the solids were washed with dry THF (2×50 mL). The combined filtrate and wash liquor was concentrated to dryness under reduced pressure and the gummy residue was dissolved in toluene (300 mL). To this solution was added amine 3 (7.7 g, 47.1 mmol) followed by the addition of solid sodium ethoxide (5.6 g, 85.6 mmol). The mixture was heated and the alcohol was distilled from the reaction mixture. After 24 h, the reaction was checked by TLC analysis and determined to be complete. The reaction mixture was cooled to room temperature and quenched into 1 N HCl solution (300 mL). The biphasic mixture was stirred for 2 h and the solid 12 was collected by filtration and washed with toluene (100 mL). The wet solid was suspended in water (100 mL) and the pH of the slurry was brought to ˜8 by the addition of saturated aqueous sodium bicarbonate solution. After stirring for 2 h, the solids were collected by filtration, washed with water and air dried. This solid was suspended in a 10:1 mixture of cyclohexane and ethyl acetate (200 mL) and heated until the solids dissolved. Decolorizing carbon was added, stirred for 30 minutes and the hot mixture clarified. The filtrate was cooled in an ice bath for 2 h and the resulting solid was collected by filtration. The solid was vacuum dried at 60° C. to afford 17.5 g of 12, in 88% yield, as an off white solid. The NMR was consistent with the structure and identical with the previously prepared material.

### Polymorphs and Hydrates of 2-[4-(2-aminoethoxy)-3-methoxyphenyl]-N-[3-(3,4-dimethylphenyl)propyl]acetamide (DA-5018)

The “polymorphs” described herein refer to pharmaceutical compounds, specifically DA-5018, having more than one crystalline form, wherein each crystalline form has different physical properties as a result of the order of the molecules in the crystal lattice. Polymorphism occurs when more than one way exists to satisfy the energy constraints imposed on molecules as they arrange into a solid made up of the lattices of the crystalline compound. The lattices of various polymorphs reveal differences in symmetry elements, spatial arrangements, and intermolecular binding. Each polymorph is a distinct thermodynamic entity since intermolecular forces contribute to the properties of a solid. Polymorphs exhibit a variety of chemical, physical, mechanical, electrical, thermodynamic, and biological properties from each other. Drugs existing in polymorphic systems, such as DA-5018, have differences in some or all of these properties: storage stability, compressibility, density, solubility, melting point, dissolution rate, chemical stability, physical stability, powder flowability, compaction, and particle morphology.

The “hydrates” described herein refer to pharmaceutical compounds, specifically DA-5018, having a specific physical form as a solid crystalline compound containing water molecules bound into, and forming an integral part of, the lattice of the crystal in a likely molar amount, possibly a sub-molar amount. The water molecules are combined in a definite ratio with the crystal. In this regard, solvates are further contemplated as examples of hydrates herein.

In preferred embodiments, the present subject matter relates to methods of identifying, obtaining, and purifying the various polymorphs and hydrates of DA-5018. These polymorphs and hydrates, namely Forms I-V, were identified as four distinct crystal forms and a solvate form. Certain physical characteristics of these polymorphs generated during crystallization studies, are as follows:
Form I: typically obtained from any isolation or purification method involving water (for instance crude DA-5018 precipitated from water, recrystallization from methanol/water or acetonitrile/water). This form displays a characteristic XRPD pattern and a DSC profile with endothermic transitions averaging 105 and 112° C. It has a propensity to absorb moisture and forms a stable dihydrate at a relative humidity of >60%, as shown by DVS studies. Less crystalline, or amorphous, samples can uptake moisture at lower Relative Humidity (approximately 30% RH). This form is a desired purity improvement, and has a good degree of recovery with methanol/water.
Form II: also called the “anhydrate form” is very crystalline, non-hygroscopic, and melts at higher temperature (114-115° C., single endothermic transition in the DSC scan). In fact, this form has the highest observed melt temperature of the five forms described herein. This form can be generated from Form I using a melt-recrystallization process. It can also be obtained in the same fashion from the dihydrate (Form III, see below), or from recrystallization of DA-5018 from ethyl acetate or isopropyl acetate.
Form III: also called the “dihydrate form” is obtained as described above from hydration of Form I. As such, this form is the dihydrate of Form I. This form is additionally believed to be a slight expansion of the water channels of Form I.
Form IV: obtained from recrystallization of DA-5018 from acetonitrile. This process has a low recovery.
Form V: obtained from recrystallization from isopropyl alcohol and methyl ethyl ketone (MEK).

Crystallization procedures were developed that afford chemical purity improvement of DA-5018 to >98%. Determinations of purity were made based on standard chromatographic methodologies. Further analysis of compounds generated during this study indicates that the polymorphic or hydrate form obtained can be controlled and consistently produced.

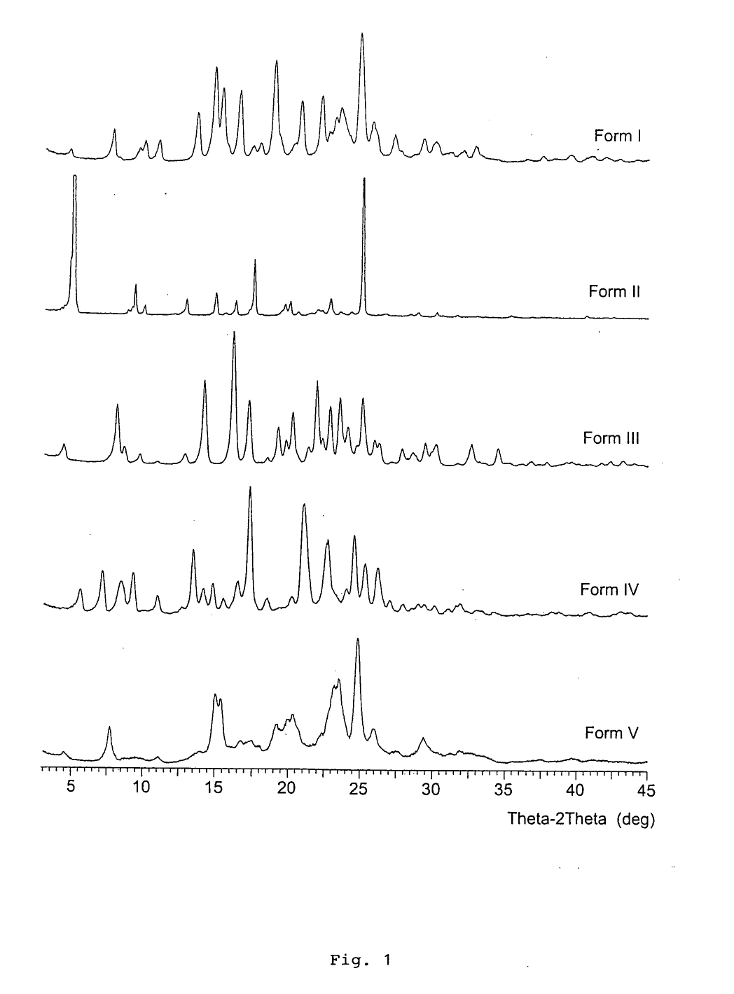

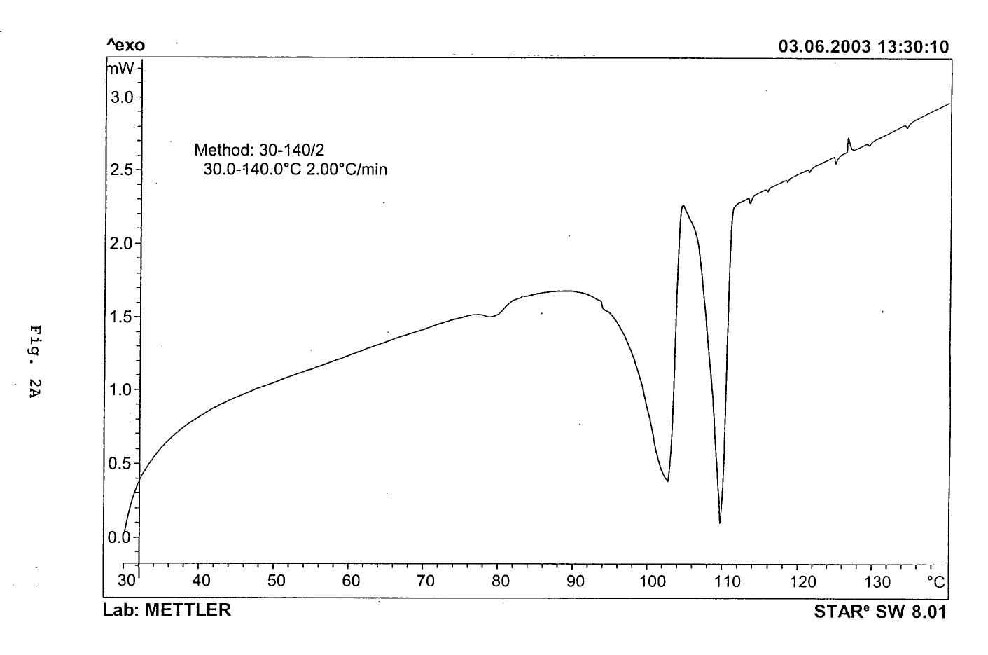

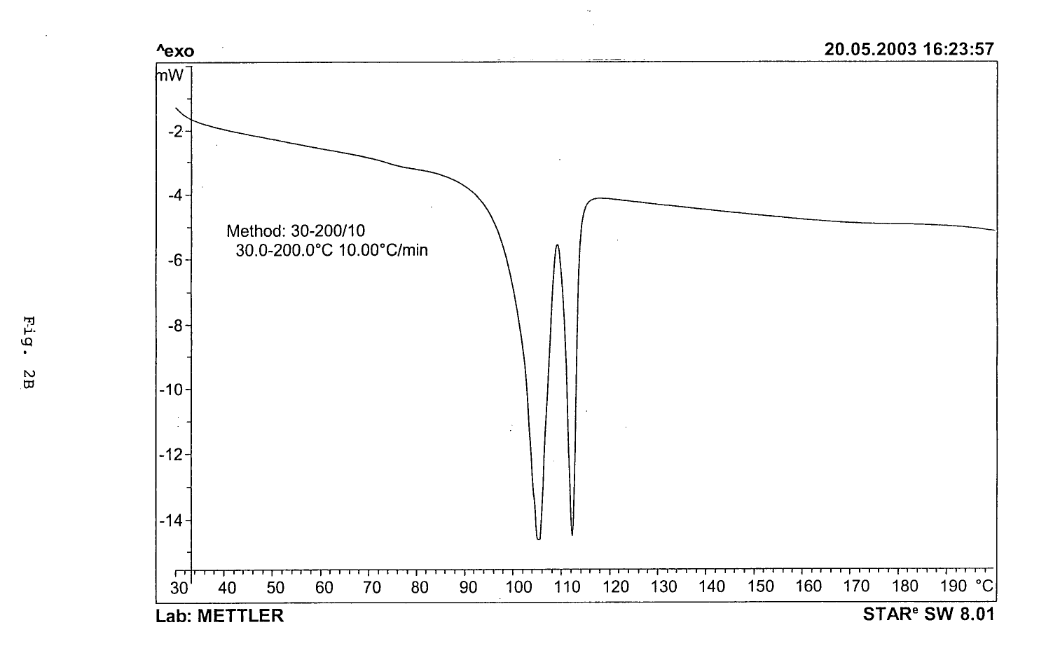

XRPD patterns presented in Table 1 and FIG. 1 demonstrate the differences of crystal lattices between the different forms. DSC data was determined to not be as selective as XRPD in differentiating forms. As presented in Table 2, endothermic transitions were observed to vary with solvent content and did not have significant temperature separation between forms. Form I was identified to be produced consistently from crystallization solvents which contained residual amounts of water. The material however was also observed to convert to Form II under thermal conditions (10 minutes of isothermal heating near melting point, 105° C.) or to a dihydrate (Form III) when exposed to moisture above 70% RH. Form II was identified to be consistently produced by exposing Form I or IV to thermal stress and melt respectively. Form III is expected to convert to Form II upon thermal stress but was not evaluated. Additionally, Form II was observed to be produced consistently from dried material using either ethyl acetate (EtOAc) or isopropyl acetate (IPAc) as a crystallization solvent. Form II was characterized as having the highest observed melt, absence of residual solvent, and no appreciable hygroscopicity at conditions up to 90% RH. Form IV was produced from acetonitrile (ACN) recrystallizations having a melting endotherm similar to Form II. Form V was observed to be generated from either isopropanol (IPA) or methyl ethyl ketone (MEK).

Crystallization methods developed demonstrated the capability to consistently produce both Form I and II with chemical purity above 98% wt. A full description of these crystallization methods is described in Examples 26 through 45. Form IV showed improved chemical purity but did not meet the pharmaceutical SPI requirements of >98% purity and recovery was low.

Representative X-ray powder diffraction (XRPD) patterns for the characteristic DA-5018 polymorphic forms are shown as a stack plot in FIG. 1. Characteristic 2-theta peaks for each of the forms were observed as presented in Table 1. Additional crystallization conditions and differential scanning calorimetry (DSC) thermal results are presented in Table 2. XRPD and DSC were observed to be definitive analytical techniques in differentiating crystalline forms and in identifying the polymorphic/hydrate unique forms observed in this study. Form II was observed to have the most intense peaks indicative of both crystallinity and preferred orientation with respect to the X-Ray beam. Form V was observed to have broad peaks indicative of amorphous material making determination of characteristic bands difficult.

TABLE 1
Characteristic XRPD 2-theta Positions for Forms Observed

| Form | XRPD 2-theta positions |
|---|---|
| I | 7.8, 11.0, 13.7, 14.9, 15.4, 16.6, |
| | 19.0, 20.8, 22.2, 25.0 |
| II | 5.0, 9.4, 12.9, 14.9, 16.3, 17.5, 22.8, |
| | 25.0 |
| III | 8.2, 14.2, 16.2, 20.2, 21.9, 22.9, |
| | 23.5, 25.1 |
| IV | 7.2, 8.5, 9.3, 13.5, 17.3, 21.1, 22.7, |
| | 24.6, 25.3, 26.2 |
| V | 7.8, 24.9 |

Representative DSC and thermal gravimetric analysis (TGA) results from unique forms are summarized in Table 2. TGA data helped distinguish physical characteristics of unique forms. Variations in DSC endotherms were observed between sample lots with similar XRPD patterns, which were attributed to residual solvent and impurities in the sample. XRPD patterns were used to identify the form being studied. Analysis of the XRPD pattern indicated that the dihydrate is a different crystal structure and is identified as Form III.

TABLE 2
Thermal Analysis of DA-5018 Polymorphs

| | | DSC | | |
|---|---|---|---|---|
| | | Onset | Peak | TGA |
| Form | Comment | (° C.) | (° C.) | (wt. %) |
| I | Methanol/100% H₂O | 103.1 | 106.1 | None |
| I | Acetonitrile/350% H₂O | 99.5 | 105.0 | 0.3 |
| | | | 111.6 | |
| I | Ethanol/150% H₂O | 84.5 | 104.2 | 0.9 |
| | | | 111.3 | |
| II | Charcoal, Methanol/140% H₂O | 113.3 | 115.0 | None |
| | recryst, dry, IPAc recryst | | | |
| II | EtOAc recrystallization | 107.9 | 111.0 | 1.5 |
| III | Exposure of DA-5018-54-1-2 | | 77.4 | 8.2 |
| | to 85% RH overnight | | 105.9 | |
| | | | 114.9 | |
| | | | 118.1 | |
| IV | Charcoal then Acetonitrile | 82.6 | 87.0 | 1.4 |
| | | | 109.4 | |
| V | IPA recrystallization | 79.7 | 91.7 | 3.3 |
| | | | 103.7 | |
| V | MEK recrystallization | | 78.5 | 4.5 |
| | | | 103.8 | |
| | | | 159.4 | |

Significant differences were observed for DSC endotherms between recrystallizations from Methanol/Water, Ethanol/Water, and Acetonitrile/Water despite having similar XRPD patterns. Additionally, the weight percent standard (DA-5018) exhibited numerous endothermic transitions as presented in Table 3.

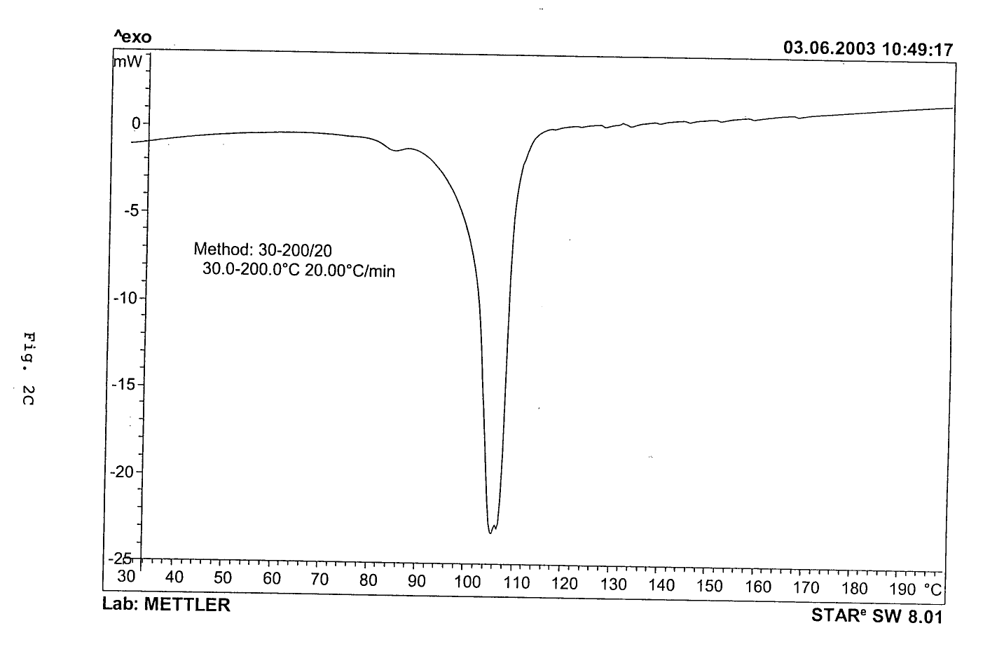

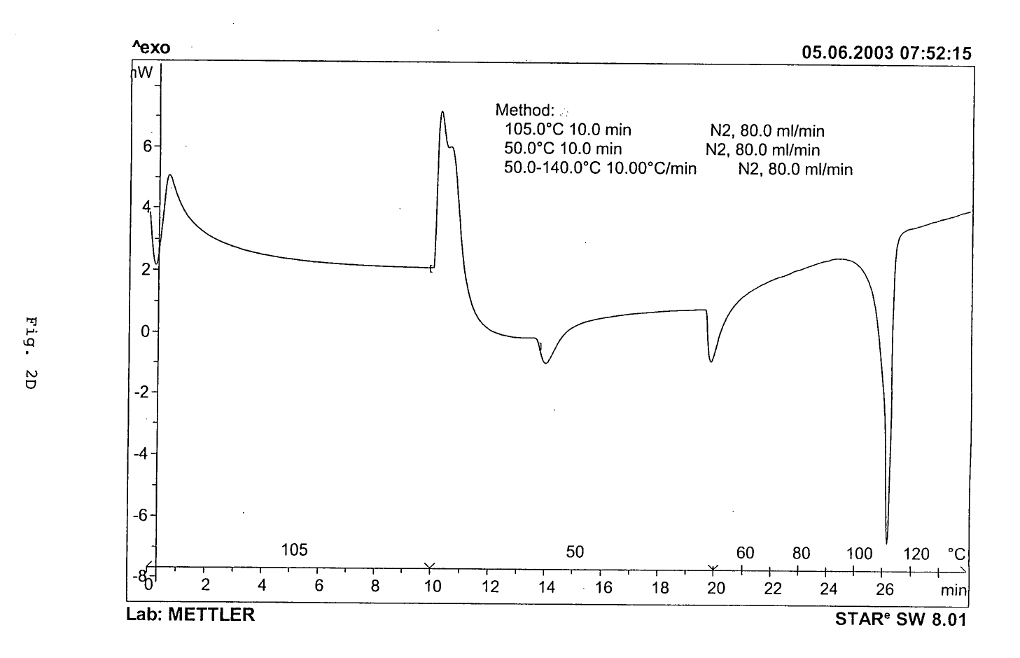

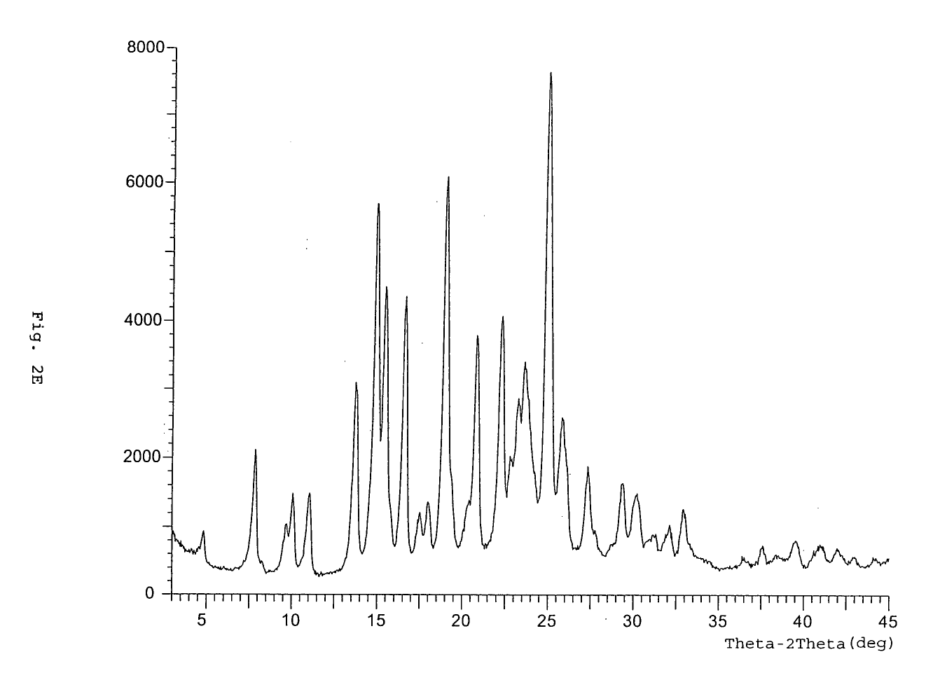

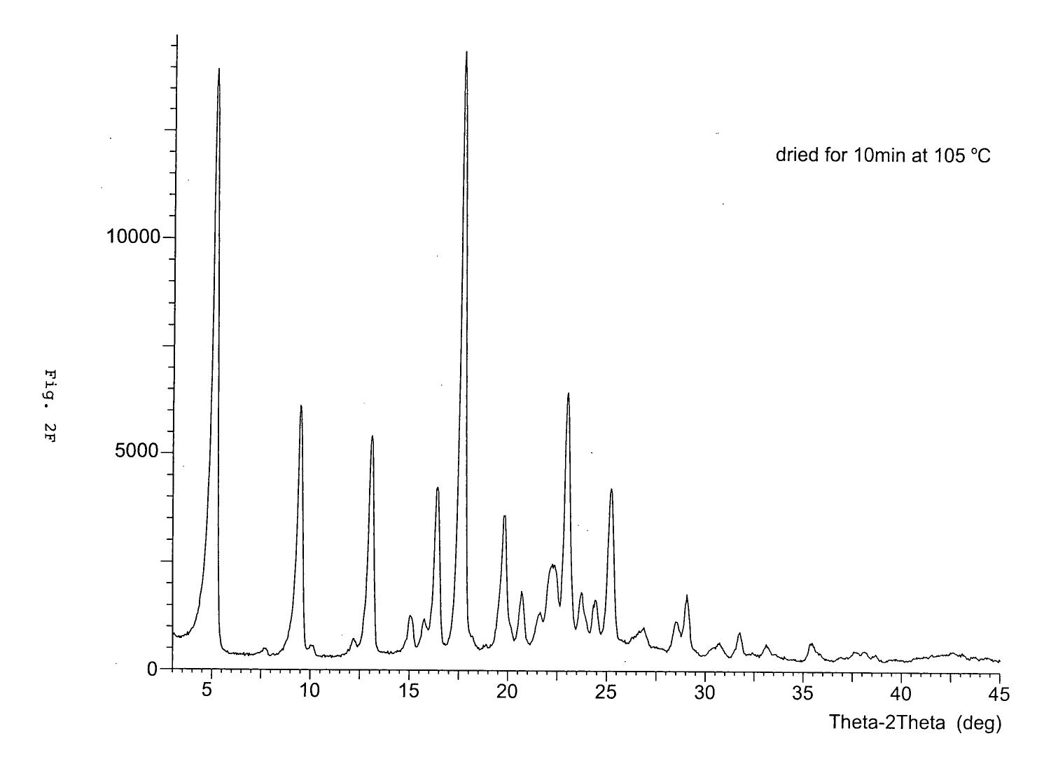

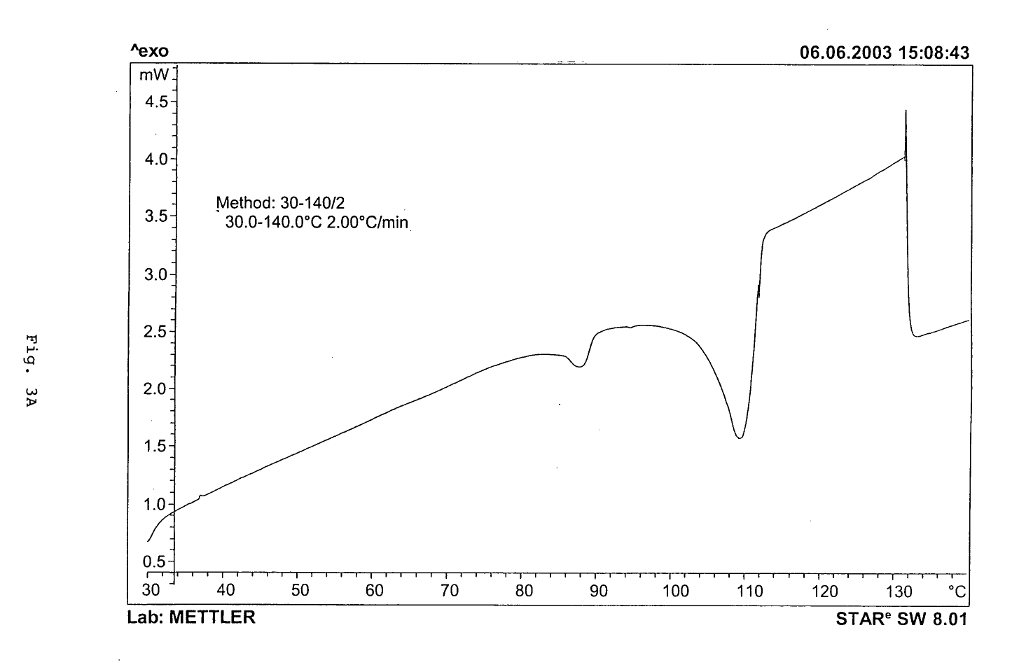

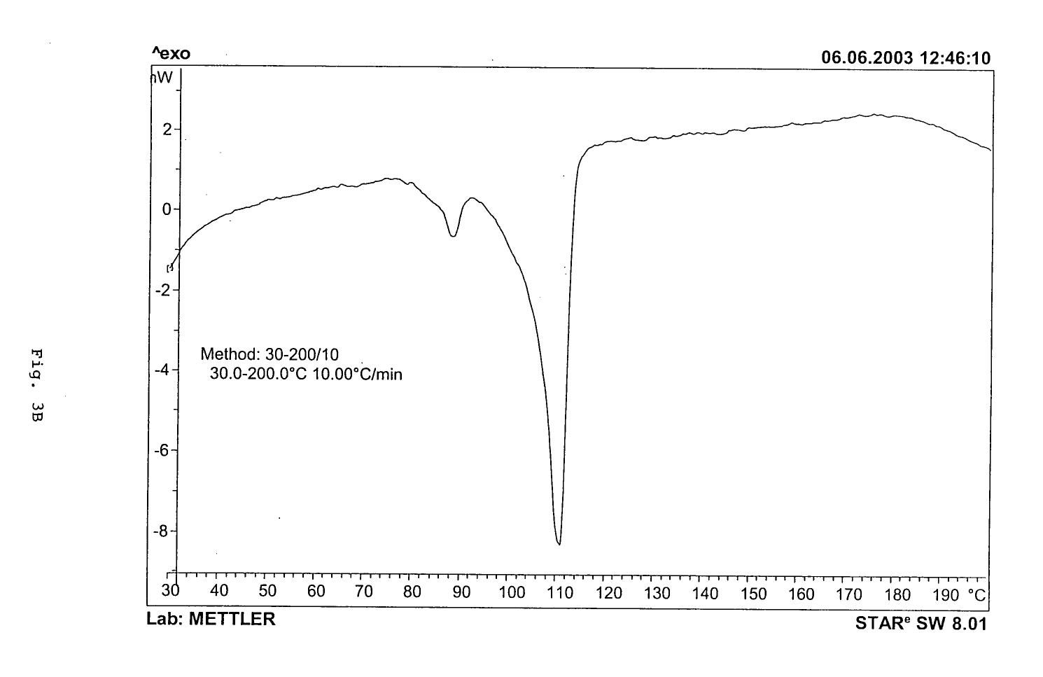

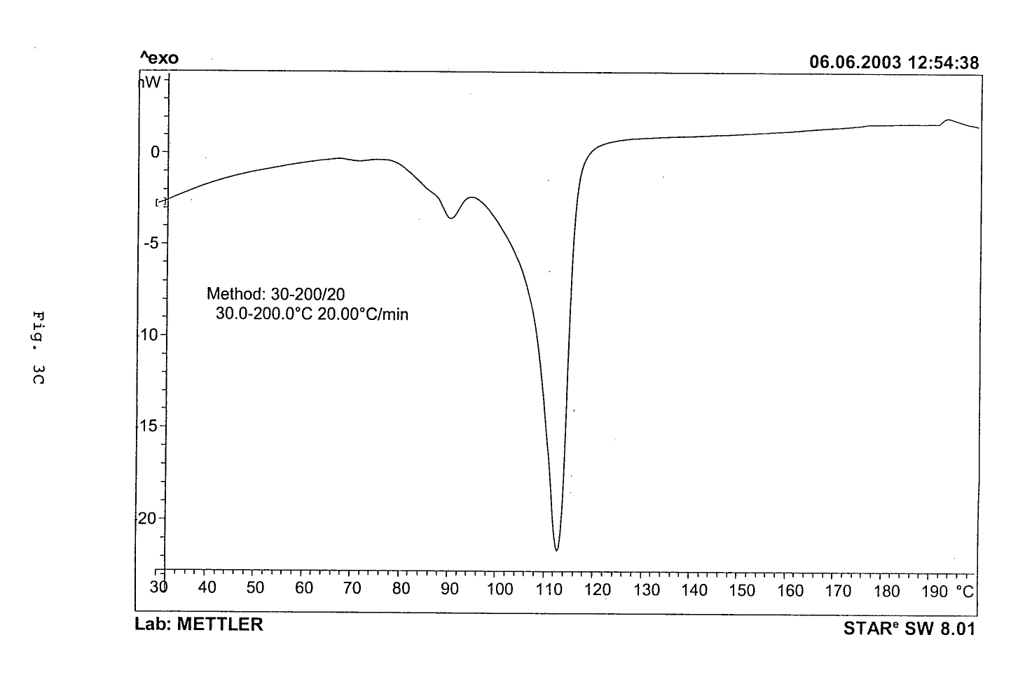

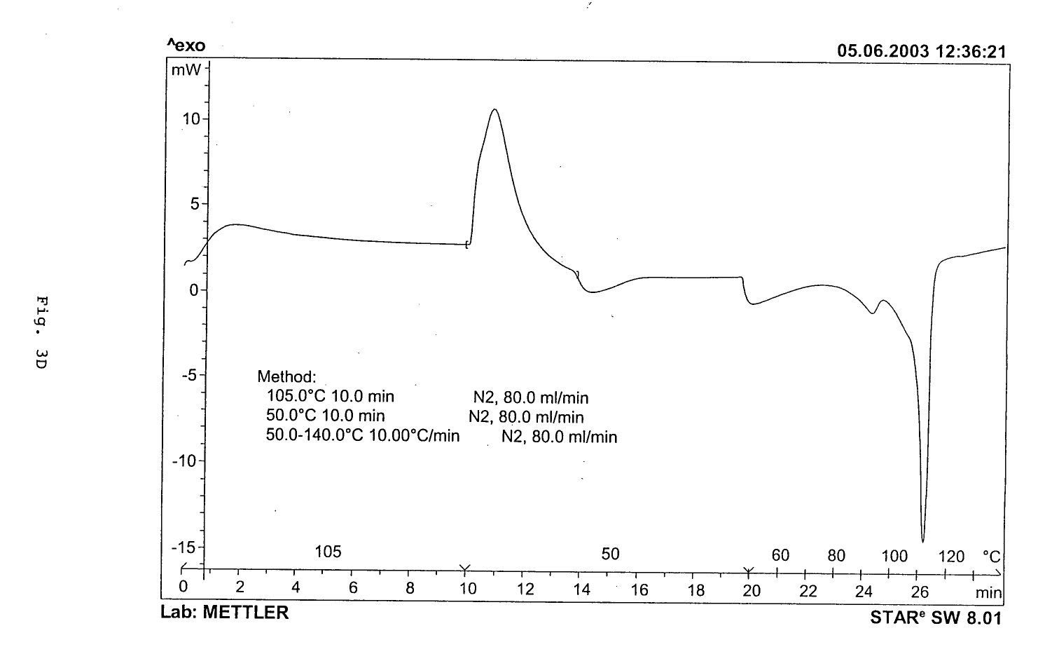

To further evaluate these observations, DSC thermograms were obtained for select compounds at various ramping rates to evaluate interconversion of forms, as shown in FIGS. 2(a)-2(c) and 3(a)-3(c). FIGS. 2(a)-2(c) show the conversion of Form I to Form II. The conversion is most readily observed in FIG. 2(a), as conversion was more difficult to observe at the faster ramp rates. Similarly, FIGS. 3(a)-3(c) show the conversion of Form IV to Form II. Additionally, isothermal heating experiments were performed with evaluation by DSC and XRPD to confirm any change of form, as shown in FIGS. 2(d)-2(f) and 3(d)-(f) and Table 3. FIGS. 2(e) and 3(e) show the original XRPD for Forms I and IV, respectively, while FIGS. 2(f) and 3(f) show the XRPD of Form II, formed after thermal conversion.

TABLE 3
Summary of Thermal Stress Results from DSC and XRPD
Experiments of DA-5018 Polymorphs

| | | DSC | | |
|---|---|---|---|---|
| Comment | DSC Exp. | Onset (° C.) | Peak (° C.) | Form |
| Methanol/350% | 2° C./min | 77.4 | 79.4 | I |
| H₂O | | | 102.6 | |
| | | | 109.6 | |
| | 10° C./min | 99.5 | 105.0 | |
| | | | 111.6 | |
| | 20° C./min | 80.9 | 84.0 | |
| | | 100.5 | 104.8 | |
| | H—C—H | 108.4 | 111.3 | II |
| Wt. % Std. | 10° C./min | 70.8 | 75.6 | I |
| | | | 99.3 | |
| | | | 106.2 | |
| | | | 111.0 | |
| | H—C—H | 113.7 | 115.3 | II |
| Acetonitrile | 2° C./min | 85.9 | 87.8 | IV |
| | | 105.0 | 109.1 | |
| | 10° C./min | | 88.3 | |
| | | | 110.5 | |
| | 20° C./min | 88.3 | 90.2 | |
| | | | 111.8 | |
| | H—C—H | 108.3 | 111.3 | IV |

H—C—H: Heat-cool-heat experiment where the compound is isothermally heated at 105° C. for 10 minutes cooled to 50° C. for 10 minutes then reheated at 10° C./min to 150° C.

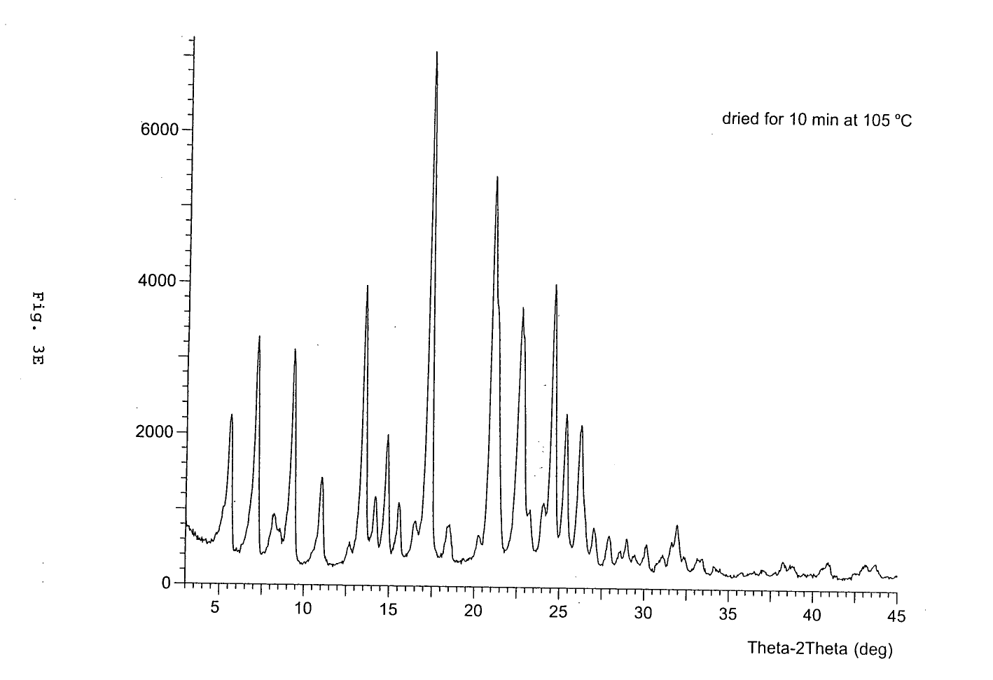

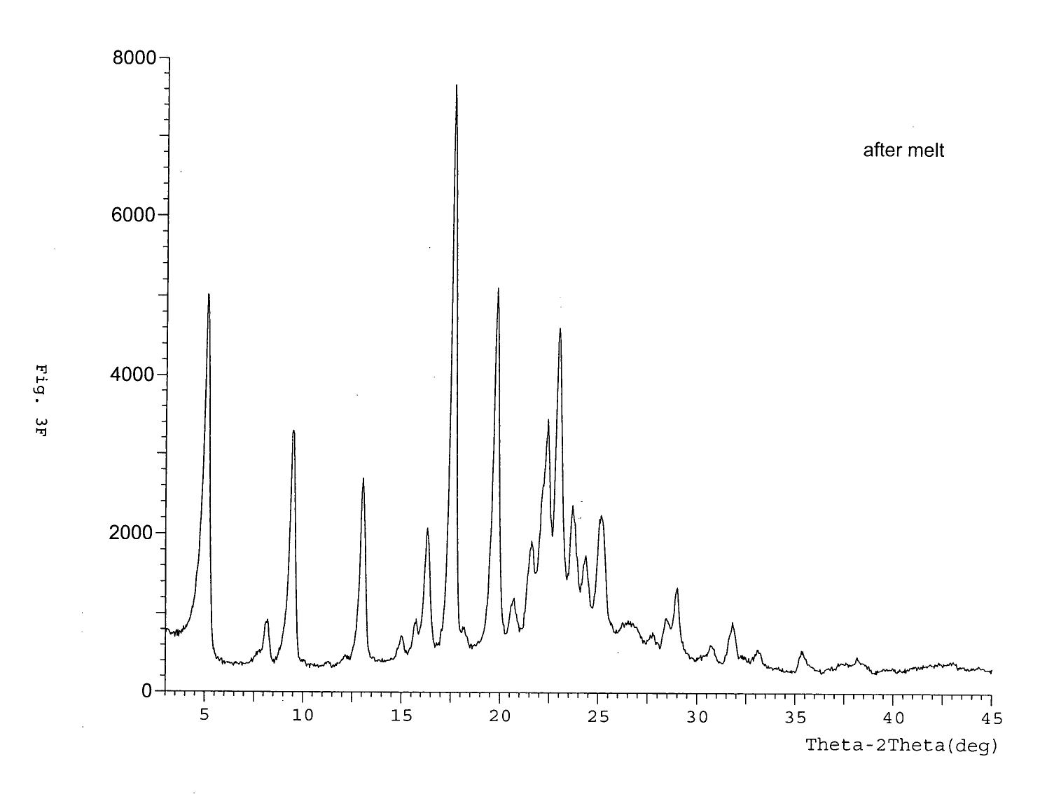

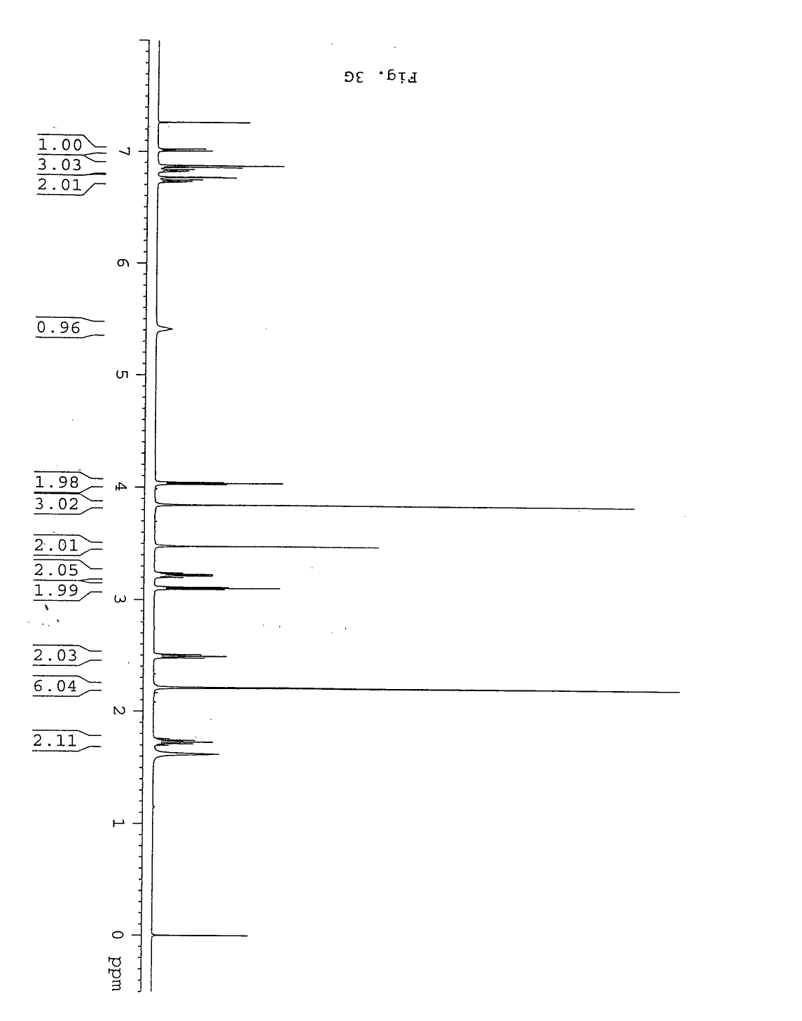

Hygroscopicity studies were conducted by DVS on both the original Form I (FIG. 4) and the thermally converted Form II (FIG. 5) of DA-5018. The original material of Form I was observed to form a dihydrate (Form III) upon exposure to moisture greater than 70% relative humidity (RH). Upon desorption, the dihydrate was observed to be stable to 30% RH. In an attempt to further evaluate the physical form of the dihydrate, a second sample of DA-5018 was converted to the dihydrate by exposing the material to 85% RH overnight in the DVS instrument.

The DSC data presented in Tables 2 and 3, above, shows two endothermic transitions with the first due to loss of water and the second consistent with a melt transition of Form I. The TGA data shows loss of 8.2% which is consistent with the dihydrate as presented in the DVS data in FIG. 4. The thermally converted material (Form II) was observed to be non-hygroscopic as the material did not gain significant weight when allowed to reach an asymptote at 90% RH. Additionally, the material did not retain moisture upon desorption.

Accordingly, in a preferred embodiment, the polymorph of the pharmaceutical compounds presented herein, specifically DA-5018, is a substantially pure polymorph of form II. In a particularly preferred embodiment, the substantially pure polymorph of form II of DA-5018 is substantially devoid of polymorphic or hydrate forms I, III, IV, or V as determined on a % weight basis. In a most preferred embodiment, the substantially pure polymorph of form II of DA-5018 has less than about 5% by weight of polymorphic or hydrate forms I, III, IV, or V as determined on a % weight basis.

In a further preferred embodiment, the DA-5018 is in the form of a crystalline solid comprising at least 95% of polymorph II defined by X-ray powder diffraction (XRPD) pattern. In a particularly preferred embodiment, the substantially pure DA-5018 in the form of a crystalline solid comprising polymorph II has characteristic X-ray powder diffraction (XRPD) 2-theta positions at 5.0, 9.4, 12.9, 14.9, 16.3, 17.5, 22.8, and 25.0.

I. Polymorph Purification Process

As shown above, polymorph form II of DA-5018 is the most desirable polymorphic form based on its beneficial crystallinity, thermal stability, and recrystallization features. Accordingly, processes for purifying polymorph form II of DA-5018 were identified. Preferred embodiments in this regard relate to a crystallization procedure which increased purity of DA-5018 while producing a consistent polymorphic form with acceptable recovery.

### EXAMPLE 26

### Purification of DA-5018 (1) as Polymorph forms I and II

Crude DA-5018 1 [7.0 g, 18.9 mmol) was suspended in methanol (140 mL, 20 vol) and stirred at ambient temperature until dissolution was complete. Activated carbon (2 g, Darco G-60) was added and the resulting suspension was stirred at ambient temperature for 40 min. The suspension was then filtered and washed with methanol (2×70 mL, 20 vol) to afford a pale yellow filtrate. The filtrate was concentrated under reduced pressure (40° C., 25 inches Hg vacuum) to about 50 mL volume. This methanol solution was diluted with water (140 mL) and the precipitated solids were isolated by vacuum filtration and washed with water (70 mL). The solid was then dried under reduced pressure at ambient temperature for 1 h and at 60° C. for 7 h to give 6.4 g of 1 (90% recovery). This material was suspended in methanol/water (1:1.4 v/v, 130 mL, 20 vol) and heated until dissolution was complete (48° C.). The batch was then seeded with polymorph form I seed crystals [2 wt %, 120 mg]. The temperature was decreased from 48 to 40 ° C. at 2°/hour, held at 40° C. for 4 h and then cooled to ambient temperature. The crystallized solid was isolated by vacuum filtration, washed with methanol/water (1:1.4 v/v, 30 mL), and then dried under reduced pressure at 65° C. for 7 hours to afford 5.9 g of 1, in 92% recovery, as a white solid as polymorph form I, as proven by DSC showing a peak at about 104 to about 112° C., as shown in Table 2. This solid was suspended in isopropyl acetate (60 mL, 10 vol) and heated to 78° C. Complete dissolution occurred at 72-73° C. The solution was cooled to ambient temperature with slow stirring at a rate of 9°/hour. The resulting solids were collected by vacuum filtration and washed with cold isopropyl acetate (2×30 mL). After vacuum drying the sample at 65° C. for 3 h, 5.6 g of DA-5018 1 polymorph Form II was obtained in 95% recovery. The NMR and IR spectra were consistent with the structure and the DSC showed a single melt endotherm of 115-117° C.

### EXAMPLE 27

### Crystallization of Polymorphs—Evaluation of Relative Solubility of DA-5018 in Common Solvents

In order to show which solvents or solvent/antisolvent combinations are appropriate for crystallization studies, the solubility of DA-5018 in twelve solvents was qualitatively evaluated on a 500 mg-scale. In these experiments, DA-5018 was suspended in 5 volumes of the appropriate solvent and stirred at ambient temperature, then incrementally heated until a complete solubilization was achieved.

TABLE 4
DA-5018 Solubility Trend

Acetone >> Methanol > THF > Ethanol, 1-Propanol > 2-
Propanol> MEK > MIBK > CH₃CN, EtOAc, IPAc >> MTBE >>> Water

Table 4 shows that DA-5018 free base was observed to be most soluble in acetone at ambient temperature, where a complete solubilization was achieved in 5 vol. Solubilization in alcoholic solvents required a moderate heating (at least approximately 28-30° C. in methanol, 35-40° C. in ethanol and 1-propanol, and 45-50° C. in 2-propanol).

Complete solubilization in tetrahydrofuran (THF) required heating to 35-40° C. DA-5018 was sparingly soluble in methyl ethyl ketone (MEK), methyl isobutyl ketone (MIBK), ethyl acetate (EtOAc), isopropyl acetate (IPAc), and acetonitrile and required heating to 70-80° C. to achieve a complete solubilization, whereas limited solubility was observed in methyl tert-butyl ether (MTBE), even at reflux.

After cooling to ambient temperature, the solids were filtered, dried, and analyzed by HPLC to determine whether any purity enhancement had been achieved or not. Results obtained in these screening experiments are outlined in Table 5.

TABLE 5
Crystallization Screening

| | | (%) Purity | |
|---|---|---|---|
| Solvent | Recovery (%) | (AUC) | Comments |
| — | — | 94.6 | DA-5018 Crude |
| | | | Production Sample |
| Methanol | — | — | No crystallization |
| Ethanol | — | — | No crystallization |
| 1-Propanol | — | — | No crystallization |
| 2-Propanol | 57 | 98.7 | Tan, pasty solid, slow |
| | | | filtration |
| Acetone | — | — | No crystallization |
| MEK | 15 | 97.1 | Light brown solid, slow |
| | | | filtration |
| MIBK | — | — | Very little |
| | | | precipitation |
| EtOAc | 71 | 94.0 | Light brown, somewhat |
| | | | granular solid |
| IPAc | 80 | 94.6 | Light brown, somewhat |
| | | | granular solid |
| Acetonitrile | 32 | 98.5 | Tan solid, slow |
| | | | filtration |
| THF | — | — | No crystallization |
| MTBE | 93 | 97.0 | Tan solid, somewhat |
| | | | slow filtration¹ |

¹Since very little solubilization was achieved in MTBE, this run was essentially a re-slurry.

The initial solvent screen showed that DA-5018 was freely soluble in methanol at 50° C. Additionally, on cooling to ambient temperature, the DA-5018 remained in solution. It is known that water is an anti-solvent for DA-5018 so it is expected that mixtures of water and methanol will provide a method of crystallization. This expectation also holds for other water miscible solvents such as ethanol or acetonitrile. Thus a series of screening experiments was carried out to evaluate aqueous solvent systems, particularly methanol/water, ethanol/water and acetonitrile/water.

### EXAMPLE 28

### Crystallization of Polymorphs—Evaluation of Relative Solubility of DA-5018

### Screening Experiments in Methanol/Water

The screening experiments were run by suspending 0.5 g of DA-5018 in 5 vol of methanol and dissolving the solids at 50° C. The appropriate quantity of water was then added and the mixtures were held at 50° C. for 30 minutes. The resulting solutions were then slowly cooled to ambient temperature with gentle stirring. After approximately 18 hours, each reaction mixture was diluted with 10 vol of the appropriate methanol/water mixture, filtered, and the solids were washed with an additional 10 vol of solvent. Each collected solid was dried under vacuum at 40° C., and analyzed by HPLC. The filtrates were also collected and analyzed by HPLC.

As shown in Table 6, crystallization from the methanol/water solution was achieved only when at least half an equivalent volume of water was added to the initial methanol solution. Further, the mass recovery was found to be proportional to the amount of water used as antisolvent. Also, a modest purity enhancement was achieved in all cases.

The screening experiments were repeated and extended to include 0.75 to 3.0 volume equivalents of water. In those instances where 1.25 and 1.50 volume equivalents of water were used, complete solubilization of the solids was achieved only by heating the initial slurry to 60° C., while partial solubilization was observed when >1.50 volume equivalents of water were used.

TABLE 6
Recrystallization Screening in Methanol/H₂O

| % H₂O | Recovery | (%) Purity | |
|---|---|---|---|
| Added | (%) | (AUC) | Comments |
| 5 | — | — | No crystallization |
| 10 | — | — | No crystallization |
| 25 | — | — | No crystallization |
| 50 | 31.2% | 97.0% | Crystals formed, pasty solid |
| | | | isolated, slow filtration |
| 75 | 48.6% | 96.7% | Crystals formed, pasty solid |
| | | | isolated, slow filtration |
| 100 | 77.0% | 97.2% | Crystals formed, small |
| | | | crystalline tan solid, faster |
| | | | filtration, good lead conditions |

As demonstrated in Table 7, all the solids obtained in these screening experiments were characterized by a single endothermic DSC transition and displayed the same XRPD pattern.

TABLE 7
Methanol/Water Studies

| | | | DSC (° C.) | | TGA |
|---|---|---|---|---|---|
| % H₂O | Recovery (%) | Purity (% AUC) | Onset | Peak | (% Wt Loss) |
| 75 | 48% | 96.7% | 104.3 | 106.5 | 0.89% |
| 100 | 77% | 97.2% | 103.1 | 106.1 | 0.00% |
| 110 | 74% | 97.8% | 102.7 | 105.5 | 0.72% |
| 125 | 63% | 98.7% | 103.3 | 105.8 | 0.90% |

### EXAMPLE 29

### Crystallization of Polymorphs—Evaluation of Relative Solubility of DA-5018

### Screening Experiments in Ethanol/Water

The recrystallization of DA-5018 from ethanol/water was also conducted. In these experiments, DA-5018 (0.5 g) was taken up in ethanol (5 vol) at 80° C., then the appropriate amount of water was added to the hot solution. The resultant mixture was then allowed to slowly cool to ambient temperature and held for eight h. Results from these experiments are summarized in Table 8, below.

TABLE 8
Recrystallization Screening in Ethanol/Water

| | | (%) | |
|---|---|---|---|
| % H₂O | Recovery (%) | Purity (AUC) | Comments |
| — | — | 94.6 | DA-5018 lot DPR-E-157 (1) |
| 20 | — | — | No crystallization |
| 40 | — | — | No crystallization |
| 80 | — | — | No crystallization |
| 100 | — | — | No crystallization |
| 120 | — | — | No crystallization |
| 150 | 35 | 95.7 | Light brown solid, slow |
| | | | filtration |

Crystallization was achieved only by adding at least 1.50 volume equivalents of water to the ethanol solution, thus indicating that DA-5018 was relatively more soluble in ethanol/water mixtures than in the corresponding methanol/water mixtures, where a recrystallization was achieved by adding 70-120% water with respect to methanol.

### EXAMPLE 30

### Crystallization of Polymorphs—Evaluation of Relative Solubility of DA-5018

### Screening Experiments in Acetonitrile/Water

Since DA-5018 is significantly less soluble in acetonitrile than in alcohols, it was initially dissolved in 10 vol of acetonitrile at 80° C., then the appropriate amount of water was added and the hot mixture was allowed to slowly cool to ambient temperature. As shown below in Table 9, no crystallization was observed when less than 2.5 volume equivalents of water were added to the initial acetonitrile solution.

TABLE 9
Recrystallization Screening in Acetonitrile/Water

| | Recovery | (%) Purity | |
|---|---|---|---|
| % H₂O | (%) | (AUC) | Comments |
| 10 | — | — | No crystallization |
| 20 | — | — | No crystallization |
| 30 | — | — | No crystallization |
| 40 | — | — | No crystallization |
| 50 | — | — | No crystallization |
| 100 | — | — | No crystallization |
| 200 | — | — | No crystallization |
| 250 | 25.0 | 97.0 | Clear solution at 80° C. |
| 300 | 31.0 | 97.6 | Clear solution at 80° C. |
| 350 | 37.5 | 97.3 | Clear solution at 80° C. |
| 400 | 32.0 | 95.5 | Partially dissolved at 80° C. |
| 500 | 15.0 | 97.7 | Partially dissolved at 80° C. |

In those instances where crystallization was achieved, large volumes of solvents were required and the mass recoveries where lower when compared to the methanol/water system. Thus, from a process throughput standpoint, the latter crystallization solvent system was more attractive and therefore used in optimization experiments.

It should be noted that during these screening experiments, the tan color present in some lots was not removed upon recrystallization from all the solvent evaluated. The tan color was readily removed upon treatment of methanol or ethanol solutions of this material with activated carbon to afford an off-white solid with 85-90% mass recovery.

Methanol/water was identified as the preferred solvent mixture based on mass recovery. Accordingly, it was necesary to develop a controlled crystallization protocol that would not only help achieve the desired purity but also would help control the physical properties of the purified material. One approach was to accurately evaluate the solubility of DA-5018 in the methanol/water solvent, generate pure seed crystals, and develop a crystallization protocol that would allow crystal growth rather than an uncontrolled, spontaneous crystallization.

### EXAMPLE 31

### Optimization of Recrystallization of DA-5018 from Methanol/Water

A standard procedure to determine the width of the metastable zone was used to determine the solubility curves of DA-5018 in methanol/water (1:1.25, v/v). In this procedure, DA-5018 (2.0 g) was suspended in 15 mL (7.5 vol) of methanol/water (1:1.25, v/v) and heated until all solids were dissolved (approximately 55° C.). The resulting solution was then very slowly cooled at a fixed rate of 0.2° C./min and the nucleation temperature recorded. The mixture was slowly reheated and the temperature of complete dissolution recorded. The solution was then iteratively diluted with a known volume of solvent and the nucleation as well as the dissolution points recorded at gradually lower concentrations to provide additional data points on the saturation curve as well as on the solubility curve. By definition, for a given concentration, the distance between the solubility curve and the saturation curve provides the metastable zone width (MSZW), in which crystal growth can be achieved upon careful seeding and controlled cooling, antisolvent addition, evaporation or pH adjustment, depending on the chosen crystallization conditions. Such a protocol is often crucial in maximizing crystal growth and curbing spontaneous, secondary nucleation that leads to uncontrolled crystallization.

As can be seen in Table 10 below, inconsistent results were observed when this procedure was implemented on DA-5018. The solubility of DA-5018 did not appear to increase as the amount of solvent was increased.

TABLE 10
Measurement of DA-5018 Solubility in
Methanol/Water (1:1.25, v/v)

| | Nucleation | Dissolution | |
|---|---|---|---|
| mL Solvent | Temperature (° C.) | Temperature (° C.) | Comments |
| 15 | 39.1 | 48.2 | Colorless solids |
| 20 | 34.6 | 47.8 | Colorless solids |
| 25 | 34.5 | 47.8 | Colorless solids |
| 30 | 34.2 | n.d.¹ | Colorless solids |

¹Not determined

Despite this anomaly, a rough estimate of the MSZW (10-13° C.) within the studied concentration range was achieved. Indeed, when the final solution was seeded with 0.5 wt % DA-5018 seeds (added as a slurry in 0.2 mL methanol/water, 1:1.25, v/v) at 47° C., crystallization proceeded as expected. The mixture was held at 47° C. for two h then cooled to 43° C. over two h. Further crystallization at 43° C. for 12 h followed by a hot filtration afforded colorless solids (1.14 g, 57% recovery, 98% AUC).

To evaluate whether the purity profile in seeded crystallizations changed significantly over time at different concentrations, three experiments were conducted using 10, 15, and 20 vol of methanol/water (1:1.25, v/v). In each case, the solids (0.43 g) were taken up in the appropriate amount of solvent and the slurries were heated to 60° C. then cooled to 47° C. and seeded with 2 wt % of DA-5018 (added as a slurry in 0.3 mL of the same solvent). Each batch was allowed to crystallize at 47° C. and sampled after 3.5 h and 6.0 h, respectively. The results are summarized in Table 11, below.

TABLE 11
HPLC Purity (% AUC) as a Function of
Concentration and Crystallization Time

| Volumes of Solvent | % AUC 3.5 h | % AUC 6.0 h |
|---|---|---|
| 10 | 97.9 | 98.1 |
| 15 | 97.1 | 97.9 |
| 20 | 97.6 | 97.7 |

The more supersaturated batch (first data point, above) crystallized faster than the other batches. However, this batch was observed to have a slightly higher purity than the others after 3.5 h. Apart from the second data point, above, where a noticeable increase in purity was observed between 3.5 and 6.0 h, no significant change in the purity profile over time was observed in the other two batches.

### EXAMPLE 32

### Re-Slurry of DA-5018 in Hot Water Followed by Recrystallization from Methanol/Water

While evaluating the recrystallization of DA-5018 from methanol/water, it was observed that higher proportions of water in the crystallization solvent helped improve purity of recovered DA-5018. When charcoal-treated DA-5018 was heated in 20 vol of water at 100° C. (external flask temperature) for 3 h then cooled to ambient temperature and filtered, a white solid was obtained that assayed at 98.5% pure DA-5018 by HPLC (AUC). The mass recovery for this experiment was 90% and the weight percent purity of this material had been increased to 97.7%. Recrystallization of this material from methanol/water (1:1.25, v/v) with seeding (0.5% wt seeds) afforded 84% recovery, with an improved wt % purity of 98.3%.

The only drawback to the hot water re-slurry procedure at temperatures exceeding 75° C. was that the emulsion obtained at high temperature upon partial dissolution and “oiling out” of the solids led to very fine solids upon cooling that filtered poorly. An alternative procedure that could avoid this “oiling out” is to initially dissolve the batch in a small amount of methanol at 50-60° C., precipitate the solids with excess water and re-slurry the batch at temperatures not exceeding 60° C.

Upon cooling, the resulting DA-5018-enriched solids were easier to filter.

### EXAMPLE 33

### Streamlined Carbon Treatment of DA-5018 Followed by Recrystallization from Methanol/Water

In order to further simplify the recrystallization procedure, a direct recrystallization of DA-5018 from filtrates derived from carbon treatment of DA-5018 was evaluated. In these experiments, the impact of seeding as well as the effect of slightly different cooling protocols on the purity and mass recovery were roughly evaluated. In all cases, crude DA-5018 was dissolved in 20 vol of methanol and treated with activated carbon (1 wt eq) at ambient temperature. Following filtration and washing with methanol (20 vol), the filtrate was concentrated by distillation under reduced pressure to remove 25-26 vol. To the residue was then added water (20 vol) while maintaining the temperature at 49-52° C. Crystallization was achieved upon slow cooling with or without seeding (see Table 12, below; all seeding carried out at 46-48° C.).

TABLE 12
Results from Single Isolation Procedures

| | | Purity¹ | |
|---|---|---|---|
| Wt % Seeds | Cooling Rate | (% AUC) | Recovery (%) |
| 0 | 42 to 20° C. for 2 h followed | 98.1 | 66 |
| | by 1 h at 20° C. | | |
| 0 | 52 to 30° C. for 7 h; 2 h at | 98.2 | 61² |
| | 30° C.; 30 to 20° C. for 1 h | | |
| | followed by 2 h at 20° C. | | |
| 1 | 48 to 30° C. for 6 h; 2 h at | 98.4 | 69³ |
| | 30° C.; 30 to 20° C. for 1 h | | |
| | followed by 2 h at 20° C. | | |
| 2 | 46 to 30° C. for 5 h; 2 h at | 98.7 | 70³ |
| | 30° C.; 30 to 20° C. for 1 h | | |
| | followed by 2 h at 20° C. | | |
| 5 | 45 to 35° C. for 5 h; 2 h at | 98.8 | 63 |
| | 35° C.; 35 to 20° C. for 1.5 h | | |
| | followed by 2 h at 20° C. | | |

¹HPLC analysis
²sample of the wet cake removed and submitted for physical characterization prior to drying
³yield corrected for amount of seeds.

As can be seen above, >98% purity (AUC) was achieved in all cases, whether the batch was seeded or not. While slightly better mass recoveries and purity were achieved in seeded crystallizations, the amount of seeds did not appear to have a dramatic impact on the purity (compare, for instance, the fourth and fifth data points, above). More importantly, wt % analysis of the material thus purified indicated a significant enhancement in purity (97-98%, wt/wt).

### EXAMPLE 34

### Recrystallization of Free Base from Isopropyl Acetate

Given the potential advantages associated with Form II, such as good flow properties and non-hygroscopicity, it was desirable to determine whether a recrystallization of DA-5018 initially purified via a methanol/water process (Form I) could be converted to Form II upon recrystallization from either ethyl acetate or isopropyl acetate. Indeed, when the recrystallization was carried out in IPAc (5-15 volumes), the higher melt form (Form II) was obtained in all cases with >90% mass recovery. Furthermore, wt % analysis of these samples indicated that in all cases >98% (wt/wt) was achieved. This ease of conversion from Form I to Form II thus provides a significant processing flexibility to generate either form if needed.

This sequence was demonstrated on a 10 g-scale to provide 77% mass recovery upon charcoal treatment and methanol/water re-slurry, 90% recovery upon recrystallization from methanol/water (98.3% wt/wt) and 91% recovery upon recrystallization from IPAc (98.5% wt/wt). This corresponds to an overall 63% recovery from crude lot.

### EXAMPLE 35

### Recrystallization of Form IV From Acetonitrile

DA-5018 (0.4 g) was suspended in acetonitrile (2.5 mL) and heated to 80° C. The resulting slightly turbid solution was allowed to slowly cool to ambient temperature and stand overnight. The white, crystalline solids that precipitated out were collected by filtration, washed with acetonitrile (2×1 mL) and dried at 45° C. under vacuum for 7 hours to afford 0.31 g of Form IV (76% recovery).

### EXAMPLE 36

### Recrystallization of Form V From Isopropyl Acetate

DA-5018 (0.5 g) was charged in a reaction tube, followed by IPAc (2.5 mL). The resulting slurry was stirred at ambient temperature, and only a partial solubilization was observed. The slurry was further incrementally heated until all solids were observed to dissolve (˜50° C.). The solution was then allowed to cool to ambient temperature and stand overnight. The solids were filtered and dried at 35° C. under vacuum for 15 hours to afford 0.29 g of Form V (57% recovery).

### EXAMPLE 37

### Recrystallization of Form V from Methyl Ethyl Ketone

DA-5018 (0.5 g) was charged in a reaction tube, followed by MEK (2.5 mL). The resulting slurry was stirred at ambient temperature, and only a partial solubilization was observed. The slurry was further incrementally heated until all solids were observed to dissolve (˜80° C.). The solution was then allowed to cool to ambient temperature and stand overnight. The solids were filtered and dried at 35° C. under vacuum for 15 hours to afford 0.08 g of Form V (15% recovery).

### EXAMPLE 38

### Alternative Purification of DA-5018 via HCl Salt Formation

Attempts were made to incorporate the HCl salt formation directly at the end of the hydrogenation reaction. A sample of crude DA-5018 was dissolved in 13 vol of 10% NMP/THF and 0.6 eq of methanesulfonic acid were added. This solution was diluted with 17 vol of water. THF was distilled from the methanesulfonate, then concentrated HCl was added. A solid precipitated out of solution and was isolated by filtration (approximately 80% mass recovery, 97.6% AUC by HPLC). Recrystallization from isopropyl acetate/ethanol (2:1, v/v, 30 vol) afforded 65% mass recovery from the crude HCl salt (52% recovery from free base), with 99.6% HPLC purity (AUC). Regeneration of the free base by precipitation from an aqueous methanol solution afforded DA-5018 as a white solid (99.7% AUC).

Another experiment using 3.5 g of crude was performed to attempt to improve the recovery. In this case, NMP was omitted. It was determined that THF is required to complete the ion exchange. If the crude lot is treated with an aqueous solution of 1.6 eq of methanesulfonic acid, the material does not dissolve. The addition of 3 vol of THF completes the dissolution. Distillation of the THF/water azeotrope at atmospheric pressure resulted in an aqueous homogenous solution of the methanesulfonic acid salt of crude DA-5018. Concentrated hydrochloric acid was added (5 eq) and after 1-2 minutes, a white solid precipitated. The solid was isolated by filtration and recrystallized from IPAc/Ethanol to give 3.5 g of a white solid (98.8% AUC). Regeneration of the free base afforded a white solid which analyzed at 98.5% pure by HPLC (AUC). The solid was placed in a vacuum oven at 45° C. to remove residual water. The mass of this sample was 2.4 g (67% overall recovery). Weight percent HPLC analysis indicated this material to be 97.2% in DA-5018.

II. Polymorph Purification Process from a Process Stream

As a point of comparison, the results of the polymorph purification processes obtained above were compared to a process for purifying polymorphs of DA-5018 through the use of a process stream.

### EXAMPLE 39

### Comparative Study of DA-5018 Purification from a Process Stream

A batch of DA-5018 was prepared from 30 g of the penultimate nitrile using the standard reduction conditions. Upon completion of the reaction, the crude reaction mixture was diluted with water (10 vol) and the catalyst was removed by filtration. The catalyst and the celite pad were washed with additional water to bring the total water added to 17 vol. The reaction mixture was split into separate portions that were worked-up under different conditions.

### EXAMPLE 40

### DA-5018 Isolation and Purification via Charcoal Treatment and Recrystallization from Methanol/Water

The first reaction portion was cooled to approximately 5° C. and treated with aqueous 2 M NaOH. DA-5018 precipitated as an off-white solid. The solid was collected and dried under vacuum at 45° C. to afford 6.8 g. Based on the initial volume of the reaction solution it can be estimated 6.8 g corresponds to an 87% yield. HPLC analysis indicated this material was 96.3% DA-5018 (AUC). This solid was then dissolved in methanol (20 vol), treated with 6.8 g of activated carbon and stirred for 30 minutes at ambient temperature. The slurry was then filtered and washed with an additional portion of methanol (20 vol). The methanol solution was then distilled at ambient pressure while mechanically stirred until 25 vol of methanol had been removed. The internal temperature of the resulting solution was 63° C. Water was then added (20 vol) and the heat source was removed. When the temperature of the reaction had reached approximately 49° C., crystallization occurred. The mixture was cooled to ambient temperature over the next two hours. The slurry was filtered to afford a flaky white solid, which was dried under vacuum at 45° C. It is important to note that this was a very facile filtration. The DA-5018 isolated in this manner was 97.3% (wt %) pure and the estimated yield was 62%.

### EXAMPLE 41

### Isolation and Purification of DA-5018 From a Process Stream via HCl Salt Formation

The second reaction portion was distilled to remove the residual THF. The solution was cooled to ambient temperature prior to the addition of 5 eq of aqueous hydrochloric acid. A white precipitate formed and was isolated by filtration. It is important to note that this was a very difficult filtration and required approximately one hour to complete. The isolated solid was then recrystallized from IPAc/ethanol (2:1) to afford 4.2 g (estimated 49% yield) of a white solid which was 98.9% pure by HPLC (AUC) analysis. This solid was then suspended in methanol (10 vol) and treated with 1.1 eq of 2 M NaOH. Upon the addition of water (20 vol) a white solid precipitated which was collected and dried under vacuum at 45° C. This process afforded 3.2 g (84% recovery from HCR salt) which was 98.4% pure by HPLC (wt %) analysis. An additional 0.8 g of HCl salt was isolated as a second crop which increased the yield of HCl salt to 59%. This material was further recrystallized from IPAc to provide the Form II polymorph. DSC analysis was consistent with Form II and the HPLC purity was further enhanced to 99.4% (wt %) (see Table 13).

TABLE 13
Results of Split Reaction

| | HPLC Purity | | |
|---|---|---|---|
| Sample Description | AUC | Wt % | Estimated Yield |
| Crude reaction mixture | 79.7% | — | — |
| Reaction mixture after IPAc | 96.0% | — | — |
| extractions | | | |
| Direct precipitation via pH | 96.3% | — | 87% |
| change | | | |
| Following charcoal and | 98.0% | 97.3% | 62% |
| recrystallization | | | |
| DA-5018 HCl salt | 99.2% | — | 59% |
| Isolated free base from HCl | >99.2% | 98.4% | 50% |
| salt | | | |
| Isolated free base from HCl | 100% | 99.4% | 45% |
| salt, recrystallized from | | | |
| IPAc | | | |

Reliable procedures that provide DA-5018 with >98% (wt/wt) purity and in consistent polymorphic forms have been developed. A charcoal treatment of a crude batch of DA-5018 followed by hot water re-slurry and recrystallization from methanol/water affords 65-70% mass recovery with >98% purity by weight (Form I). Recrystallization of this material from IPAc converts Form I to the higher melt, anhydrate form (Form II), with further enhancement in chemical purity, as indicated by wt % analysis. Purification of DA-5018 via the corresponding HCl salt affords an effective way to produce material with the highest purity.

IV. Additional Purification Processes

Accordingly, alternative purification procedures have been proposed as a further point of comparison. All purification procedures described herein are intended to be contemplated within the scope of the present subject matter.

### EXAMPLE 42

### Hot Water Re-Slurry Followed by Recrystallization of DA-5018 from Methanol/Water

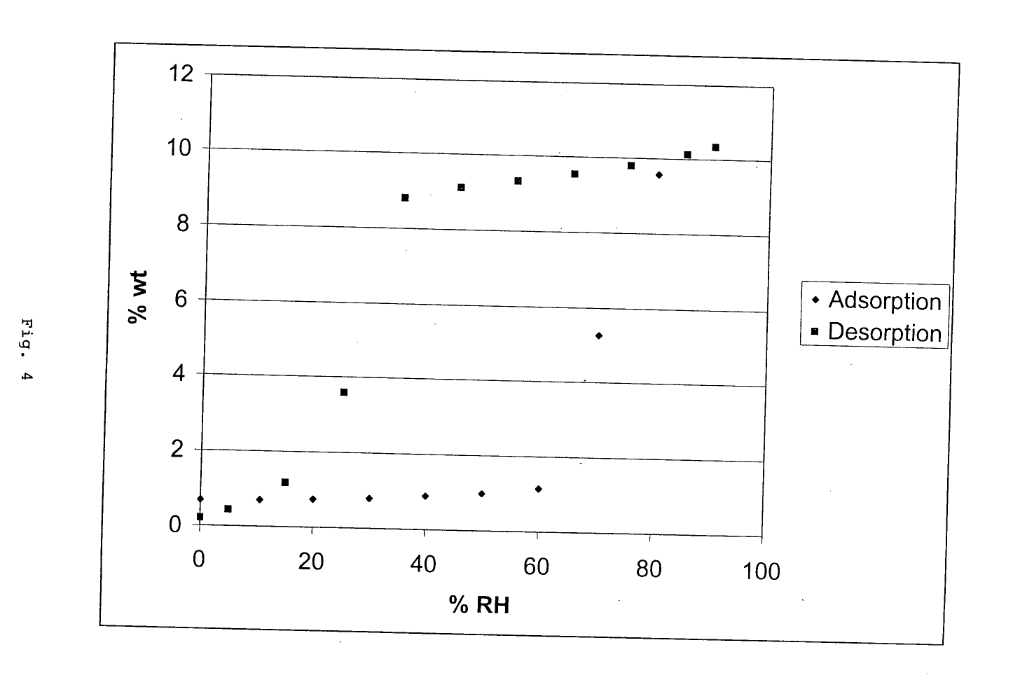

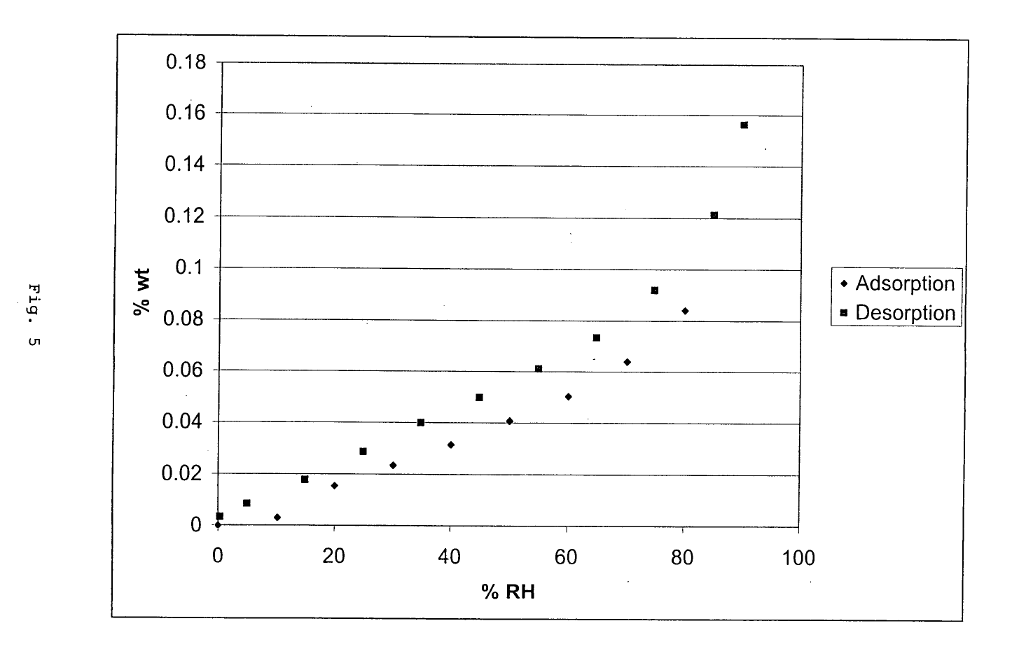

A slurry of DA-5018 (0.50 g, 88% wt % purity) in water (20 mL, 40 vol) was warmed in a sand bath at an external temperature of 100° C. After heating for 3 h, the slurry was cooled to ambient temperature. The solid was isolated by vacuum filtration and dried in a vacuum oven at 45° C. to afford 0.44 g of a white solid. HPLC analysis of this material indicated the purity to be 97.7% on a weight percent basis (see FIG. 6).

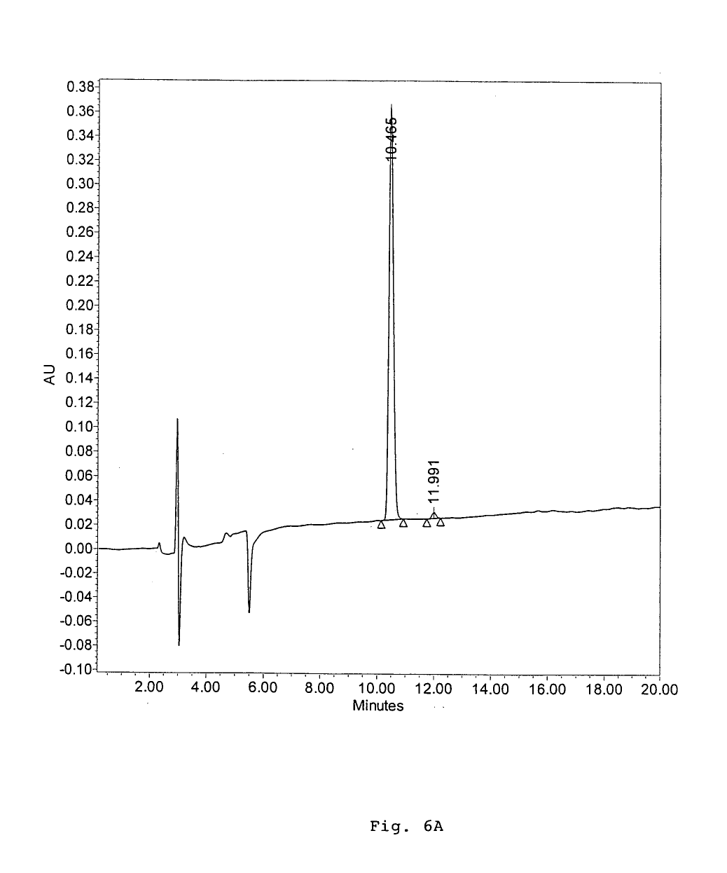

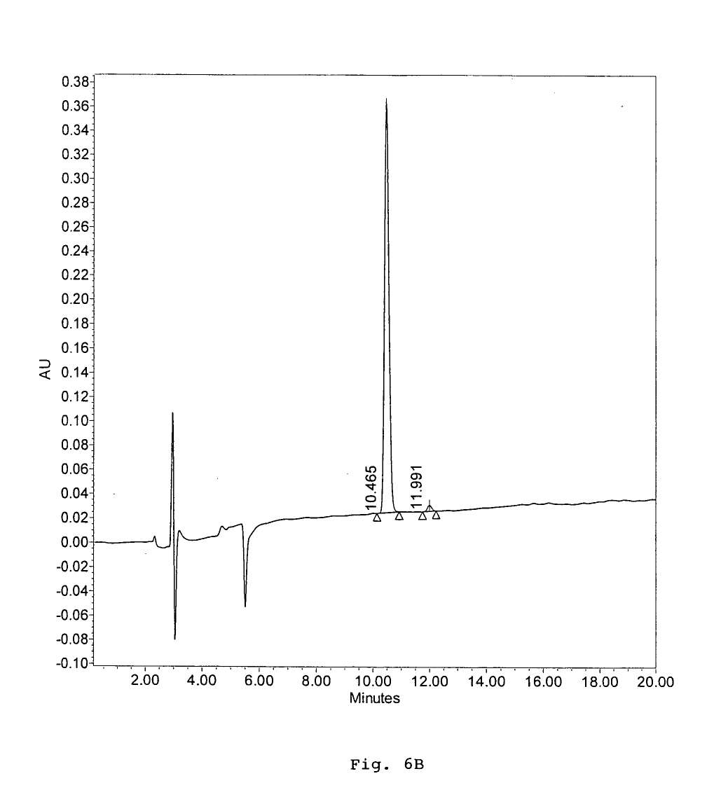

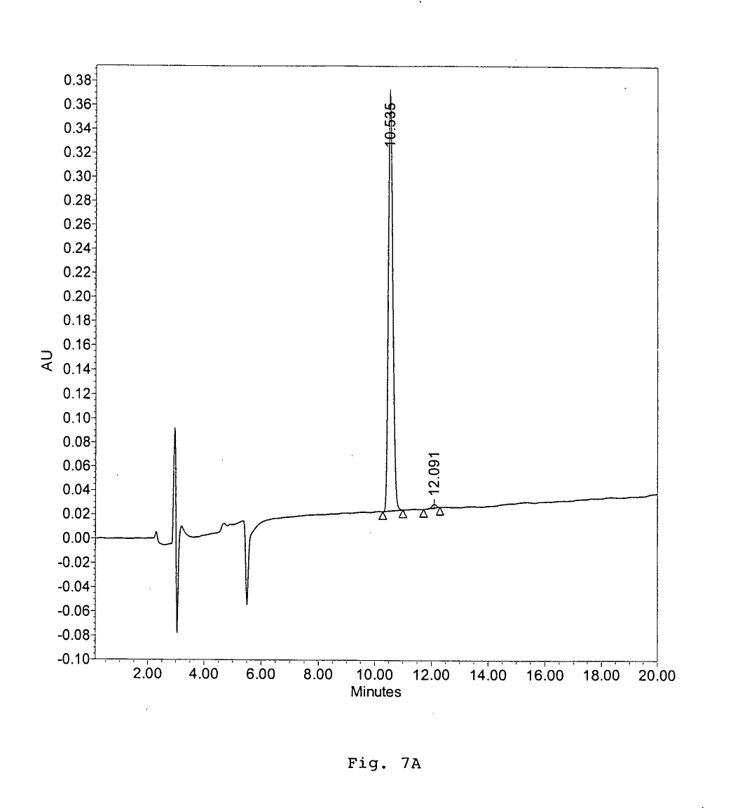

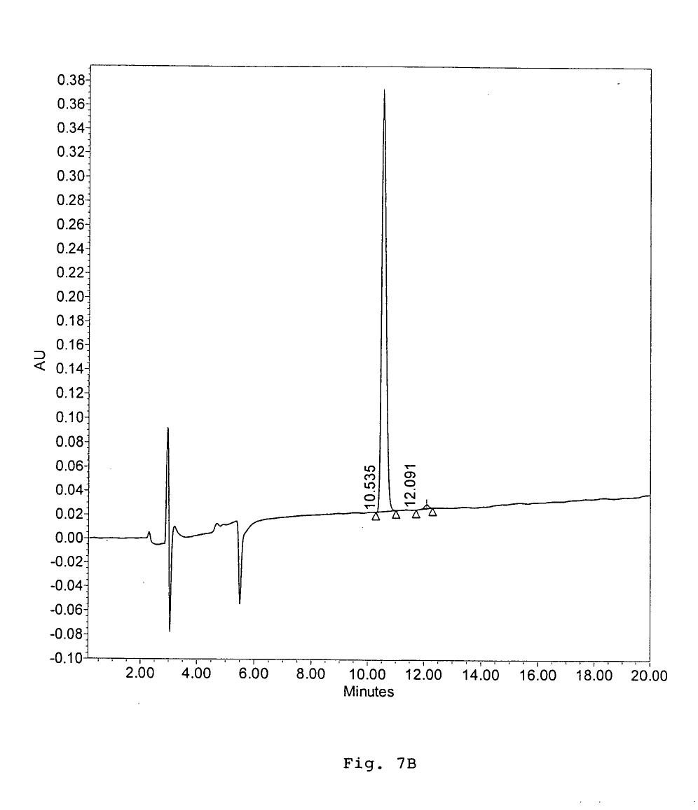

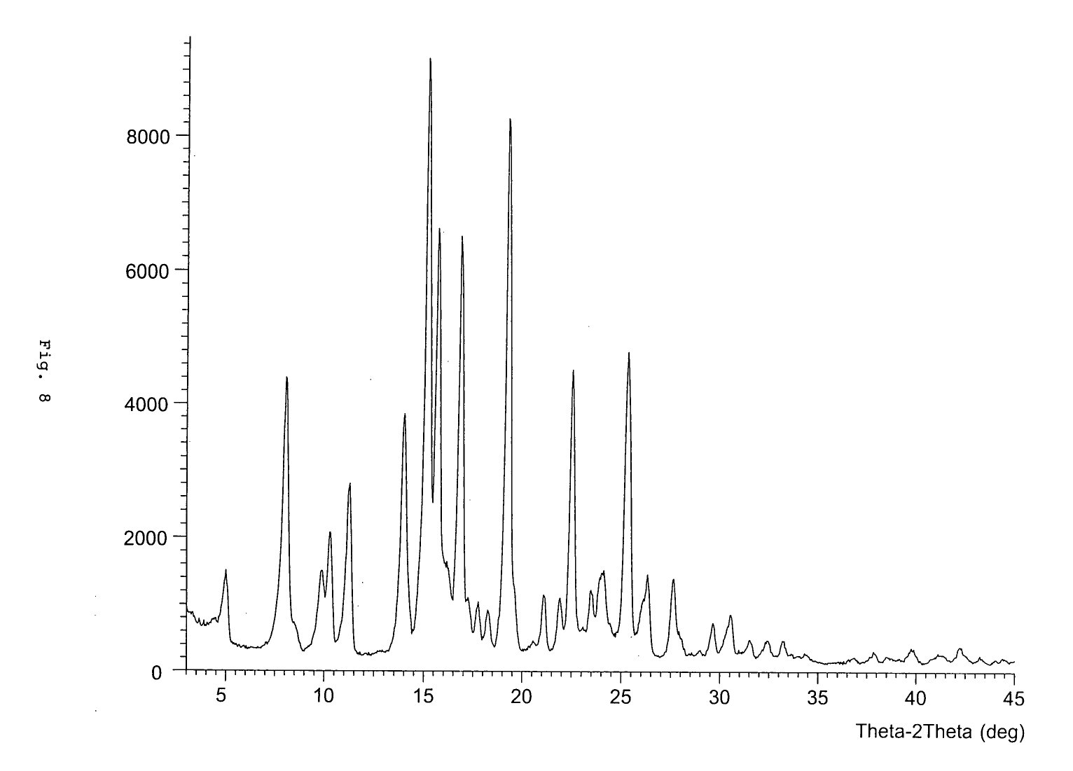

DA-5018 (0.39 g of the above batch) was suspended in methanol/water (1:1.25, v/v, 5.9 mL) in a round-bottom flask equipped with a magnetic stirring bar, a thermocouple, and a reflux condenser and heated to 60° C. to give an almost colorless solution. The batch was cooled to 47° C. and seeded with 2 mg of seed crystals added as a slurry in methanol/water (1:1.25, v/v, 0.1 mL). The batch was then set to cool to 30° C. at a rate of 3° C./h (approximately 5.5 h cooling time) and held at 30° C. for an additional 7 h. The warm slurry was filtered, partially dried at ambient temperature under reduced pressure for 30 min, and further dried to constant weight under high vacuum (5 Torr) at 50° C. for 22 h to afford a white crystalline solid in 84% yield (0.33 g). Weight % analysis of this material indicated that it was 98.3% pure (see FIGS. 7-8).

### EXAMPLE 43

### Decolorization of DA-5018 with Activated Carbon Followed by Recrystallization of DA-5018 from Methanol/Water

DA-5018 (3.5 g) was dissolved in methanol (70 mL) at ambient temperature and treated with activated carbon (Darco G-60, 3.5 g) for 30 min. The suspension was filtered to give a very pale yellow solution and washed with methanol (70 mL) and the combined filtrate concentrated under reduced pressure on a rotary evaporator (40° C., 25 inches Hg) to remove 95 mL of methanol. The residual solution (49 mL) was charged into a 250 mL, round-bottom flask equipped with an overhead mechanical stirrer, a thermocouple, and a reflux condenser and heated to 52° C. using a heating mantle.

Deionized water (70 mL) was added in small portions while maintaining the solution temperature between 49 and 52° C. The batch was cooled to 47° C. and seeded with 5 wt % seed crystals (175 mg slurry in methanol/water, 1:1.4, v/v, 4.5 mL) and cooled to 35° C. at a rate of 2° C./min, held at 35° C. for 2 h and allowed to cool to ambient temperature. The resulting white slurry was filtered, the wet cake washed with methanol/water (1:1.4 v/v, 18 mL), dried at ambient temperature under reduced pressure for 4 h, and further dried under high vacuum (5 Torr) at 65-67° C. for 2.5 d to afford DA-5018 as a white crystalline solid with 63% mass recovery (2.4 g; yield corrected for amount of seed crystals).

### EXAMPLE 44

### Recrystallization of DA-5018 as Form II

DA-5018 (1.5 g) was suspended in IPAc (22.5 mL, 15 vol) and the mixture was heated to 78° C. Complete dissolution was achieved at 68° C. Stirring of the solution was stopped to minimize breakage of the solids and the solution was cooled to 21° C. at a rate of 9° C./min. The colorless, elongated solids that crystallized were isolated by vacuum filtration and washed with IPAc (2×6 mL). Residual solvent was removed at ambient temperature under reduced pressure for 15 min and further dried to constant weight under high vacuum (5 Torr) at 65° C for 4 h to afford colorless solids in 90% recovery (1.4 g). Weight % analysis of this material indicated that it was 98.5% pure.

### EXAMPLE 45

### Purification of DA-5018 on 10-g Scale

Crude DA-5018 (10.0 g) was suspended in methanol (200 mL, 20 vol) and stirred at ambient temperature until dissolution was complete. Activated carbon (10 g, Darco G-60) was added and the resulting suspension was stirred at ambient temperature for 40 min. The suspension was then filtered and washed with an additional portion of methanol (200 mL, 20 vol) to afford a pale yellow filtrate. The filtrate was concentrated under reduced pressure (40° C., 25 inches Hg) to remove 350 mL of methanol. The solution was then mechanically stirred during the addition of water (200 mL, 20 vol) as solids precipitated. The suspension was heated to 65° C., held at that temperature for 30 min and then cooled to ambient temperature. The precipitated solids were isolated by vacuum filtration and washed with water (50 mL). The solid was then dried at ambient temperature for 1 h and at 60° C. for 7 h to give 7.67 g (77% recovery). This material was suspended in methanol/water (1:1.4 v/v, 153 mL, 20 vol) and heated until dissolution was complete (48° C.). The batch was then seeded (2 wt % , 0.153 g). The temperature was decreased from 48-40° C. at 2°/hour, held at 40° C. for 4 h and then cooled to ambient temperature. The crystallized solid was isolated by vacuum filtration, washed with methanol/water (1:1.4 v/v, 30 mL), and then dried at 65° C. for 7 h under vacuum to give 90% recovery of white solid (6.9 g). Weight % analysis of this material indicated that it was 98.3% pure. The solid was suspended in IPAc (68 mL, 10 vol) and heated to 78° C. Complete dissolution occurred at 72-73° C. The solution was cooled to ambient temperature with slow stirring at a rate of 9° C./hour. The resulting solids were collected by vacuum filtration and washed with IPAc (2×27 mL). After drying the sample at 65 ° C. under vacuum (5 Torr) for 3 h, 90% recovery (62% overall recovery) of DA-5018 was obtained (6.15 g). Weight % analysis of this material indicated that it was 98.5% pure. The melting point was measured at 115-117° C. by DSC analysis, indicating Form II.

### Salts, Bases, Amides, Solvates, Hydrates

The compounds made by the processes herein may additionally be produced, and made available in, the form of their “pharmaceutically acceptable free bases, salts, amides, or solvates”. As used herein, this phrase refers to free bases, salts, amides, or solvates of subject compound(s) which possesses the same pharmacological activity as the subject compound(s) and which are neither biologically nor otherwise undesirable. A salt, amide, or solvate can be formed with, for example, organic or inorganic acids.

Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, arginine, lysine, and so forth naturally and synthetically derived amino acids.

Non-limiting examples of base salts, amides, or solvates include ammonium salts; alkali metal salts, such as sodium and potassium salts; and alkaline earth metal salts, such as calcium and magnesium salts. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; halides, such as benzyl and phenethyl bromides; and others. Water or oil-soluble or dispersible products are thereby obtained.

### Methods of Treatment

The compounds prepared by the processes described herein, and compositions containing the same, are preferably administered to a patient in therapeutically effective amounts to treat a patient who is suffering from a disease or disorder.

In particular, capsaicinoid compounds as provided herein may be used to treat a variety of skin diseases including skin diseases diagnosed by a medical professional, such as a dermatologist. See in this regard the Manual of Skin Diseases, 6^{th} edition by Gordon Sauer, MD, 1991, J. B. Lippincott Company, Philadelphia, Pa., the disclosure of which is hereby incorporated by reference in its entirety, for a non-exclusive listing of such skin diseases.

Skin diseases involving the epidermis and dermis are of particular interest. General skin diseases treatable herein include, but are not limited to, neuralgias, inflammatory disorders, pruritis, hyperproliferative skin diseases, diseases involving skin metabolism, infections, excretions, improvement in the skin appearance and health, and combinations thereof.

More specifically, the diseases treatable herein include, but are not limited to, post herpetic neuralgia, pruritis, pruritis associated with atopic dermatitis, acne, rosacea, atopic dermatitis, psoriasis, eczema, seborrheic dermatitis, pyodermas, neurodermatitis, intertrigo, pruritis, tinea infections, verrucum, warts, viral infections, herpes simplex infections, impetigo, and combinations thereof. These skin disorders may exhibit an observable symptom selected from the group consisting of inflammation, erythema, swelling, pain, pruritis, cell hyperproliferation, telangiectasia, pyoderma, hyperpigmentation, bacterial fungal or viral infection, skin lesions, redness, pustules, cysts, nodules, papules, hypertrophy of the sebaceous glands, and combinations thereof.

In another preferred embodiment, the present methods of treatment result in an improvement of the patient's condition, reduction of symptoms, an improvement in the patient's appearance, or combinations thereof.

### Dosages

Appropriate dosage levels of any of the active ingredients presented herein, e.g. the capsaicinoids, are well known to those of ordinary skill in the art. Dosage levels on the order of about 0.001 mg to about 5,000 mg per kilogram body weight of the active ingredient compounds or compositions thereof are useful in the treatment of the above diseases, disorders, and conditions. Typically, this effective amount of the present active ingredients will generally comprise from about 0.1 mg to about 1,000 mg per kilogram of patient body weight per day. The amount of active ingredient that may be combined with the carrier materials to produce a single dosage form will vary depending upon the disease and the patient treated and the particular mode of administration. Typically, in vitro dosage-effect results provide useful guidance on the proper doses for patient administration. Studies in animal models are also helpful. The considerations for determining the proper dose levels are well known in the art.

Moreover, it will be understood that this dosage of active therapeutic agents can be administered in a single or multiple dosage units to provide the desired therapeutic effect.

The present compounds and/or compositions may be given in a single or multiple doses daily. In a preferred embodiment, the present compounds and/or compositions are given from one to three times daily. Starting with a low dose twice daily and slowly working up to higher doses if needed is a preferred strategy. The amount of active ingredients that may be combined with the carrier materials to produce a single dosage form will vary depending upon the host treated, the nature of the disease, disorder, or condition, and the nature of the active ingredients.

It is understood, however, that a specific dose level for any particular patient will depend upon a variety of factors well known in the art, including the activity of the specific compound employed; the age, body weight, general health, sex and diet of the patient; the time of administration; the rate of excretion; drug combination; the severity of the particular disorder being treated; and the form of administration. One of ordinary skill in the art would appreciate the variability of such factors and would be able to establish specific dose levels using no more than routine experimentation.

The optimal pharmaceutical formulations will be determined by one skilled in the art depending upon considerations such as the particular drug or drug combination and the desired dosage. See, for example, Remington's Pharmaceutical Sciences, 18th Ed., Mack Publishing Co., Easton, Pa 18042 (1990) and Harry's Cosmeticology, 8th Ed., Chemical Publishing Co., Inc., New York, N.Y. 10016 (2000), the entire disclosures of which are hereby incorporated by reference. Such formulations may influence the physical state, stability, rate of in vivo release, and rate of in vivo clearance of the therapeutic agents.

### Pharmaceutical Carriers

The phrase “pharmaceutically acceptable carrier” as used herein refers to any inactive ingredient present in one of the herein described compositions in an amount effective to enhance the stability, effectiveness, or otherwise of said composition. Non-limiting examples of such pharmaceutically acceptable carriers include diluents, excipients, suspending agents, lubricating agents, adjuvants, vehicles, delivery systems, emulsifiers, disintegrants, absorbants, adsorbents, preservatives, surfactants, colorants, flavorants, emollients, buffers, pH modifiers, thickeners, water softening agents, humectants, fragrances, stabilizers, conditioning agents, chelating agents, sweeteners, propellants, anticaking agents, viscosity increasing agents, solubilizers, plasticizers, penetration enhancing agents, glidants, film forming agents, fillers, coating agents, binders, antioxidants, stiffening agents, wetting agents, or any mixture of these components.

The carriers useful herein further include one or more compatible solid or liquid filler, diluents, or encapsulating materials which are suitable for human or animal administration.

The biocompatible carriers, as used herein, are the components that do not cause any interactions which substantially reduce the efficacy of the pharmaceutical composition in an ordinary user environment. Possible pharmaceutical carriers must be of sufficiently low toxicity to make them suitable for administration to the subject of treatment.

Some examples of substances which can serve as a carrier herein are sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa buffer (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, tabletting agents, stabilizers, antioxidants, and preservatives may also be present.

Any non-toxic, inert, and effective carrier may be used to formulate the compositions contemplated herein. Pharmaceutically acceptable carriers, excipients, and diluents in this regard are well known to those of skill in the art, such as those described in The Merck Index, 13th Ed., Budavari et al., Eds., Merck & Co., Inc., Rahway, N.J. (2001); the CTFA (Cosmetic, Toiletry, and Fragrance Association) International Cosmetic Ingredient Dictionary and Handbook, 10th Ed. (2004); and the “Inactive Ingredient Guide”, U.S. Food and Drug Administration (FDA) Center for Drug Evaluation and Research (CDER) Office of Management, January 1996, the contents of which are hereby incorporated by reference in their entirety. Examples of preferred pharmaceutically acceptable excipients, carriers and diluents useful in the present compositions include distilled water, physiological saline, Ringer's solution, dextrose solution, Hank's solution, and DMSO.

These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990) and Remington's Pharmaceutical Sciences, 18th Ed., Mack Publishing Co., Easton, Pa. (1990), both of which are incorporated by reference herein in their entirety.

The carrier may comprise, in total, from about 0.1% to about 99.99999% by weight of the pharmaceutical compositions presented herein, mainly from about 50% to about 99.9999%.

The topical compositions contemplated herein, may take the form of a gel, cream, lotion, suspension, emulsion, aerosol, ointment, foam, shampoo, tablet, capsule, mixtures thereof, or any other pharmaceutical dosage form commonly known in the art. Other pharmaceutical and cosmetic treatment compositions known to those skilled in the art, including liquids and balms, are additionally contemplated as falling within the scope of the present subject matter. Further, the present subject matter contemplates applying any of these compositions with an applicator. Non-limiting examples of useful applicators include a pledget, a pad, and combinations thereof. Additionally, the present subject matter further contemplates that any of these topical compositions are provided in a package of less than 5 g topical composition as a unit of use.

Emulsions, such as oil-in-water or water-in-oil systems, as well as a base (vehicle or carrier) for the topical formulation is selected to provide effectiveness of the active ingredient and/or avoid allergic and irritating reactions (e.g., contact dermatitis) caused by ingredients of the base or by the active ingredients.

Creams useful herein may also be semisolid emulsions of oil and water. They are easily applied and vanish when rubbed into the skin.

Lotions useful herein include suspensions of powdered material in a water or alcohol base (e.g., calamine), as well as water-based emulsions (e.g., some corticosteroids). Convenient to apply, lotions are also cool and help to dry acute inflammatory and exudative lesions.

Suitable lotions or creams containing the active compound may be suspended or dissolved in, for example, a mixture of one or more of the following: mineral oil, sorbitan monostearate, polysorbate 60 (polyoxyethylene 20 sorbitan monostearate), cetyl ester wax, cetearyl alcohol, 2-octyldodecanol, benzyl alcohol, and water.

Ointments which are useful herein are oleaginous and contain little if any water; feel greasy but are generally well tolerated; and are best used to lubricate, especially if applied over hydrated skin. These ointments are preferred for lesions with thick crusts, lichenification, or heaped-up scales and may be less irritating than cream formulations for some eroded or open lesions (e.g., stasis ulcers). Drugs in ointments are often more potent than in creams.

The compounds can be formulated into suitable ointments containing the compounds suspended or dissolved in, for example, mixtures with one or more of the following: mineral oil, liquid petrolatum, white petrolatum, propylene glycol, polyoxyethylene polyoxypropylene compound, emulsifying wax and water.

In severe cases, occlusive therapy may be useful herein. Covering the treated area with a nonporous occlusive dressing can increase the absorption and effectiveness of the compounds described herein. Usually, a polyethylene film (plastic household wrap) is applied overnight over cream or ointment, since a cream or ointment is usually less irritating than lotion in occlusive therapy. Plastic tapes may be impregnated with drug and are especially convenient for treating isolated or recalcitrant lesions; children and (less often) adults may experience pituitary and adrenal suppression after prolonged occlusive therapy over large areas.

Suitable gelling agents which may be useful in the present compositions include aqueous gelling agents, such as neutral, anionic, and cationic polymers, and mixtures thereof. Exemplary polymers which may be useful in the instant compositions include carboxy vinyl polymers, such as carboxypolymethylene. A preferred gelling agent is Carbopol® brand polymer such as is available from Noveon Inc., Cleveland, Ohio. Carbopol® polymers are high molecular weight, crosslinked, acrylic acid-based polymers. Carbopol® homopolymers are polymers of acrylic acid crosslinked with allyl sucrose or allylpentaerythritol. Carbopol® copolymers are polymers of acrylic acid, modified by long chain (C10-C₃₀) alkyl acrylates, and crosslinked with allyl-pentaerythritol.

Other suitable gelling agents include cellulosic polymers, such as gum arabic, gum tragacanth, locust bean gum, guar gum, xanthan gum, cellulose gum, methylcellulose, hydroxyethylcellulose, hydroxypropylcellulose, and hydroxypropylmethylcellulose.

### Additional Active Ingredients

The subject matter described herein further contemplates administering an additional active ingredient, other than those above described, readily known to those of skill in the art as useful in the treatment of any of the diseases, disorders, or conditions herein described. These additional active ingredients are administered topically or orally either concomitantly or sequentially with the above described compounds and/or compositions. Accordingly, the additional active ingredient is administered with the compound and/or composition either in adjunctive or co-therapy. That is, the additional active ingredient can either be administered as a component of the composition or as part of a second, separate composition. This second, separate composition can be either an oral or a topical composition.

Exemplary additional active ingredients include, but are not limited to, macrolide antibiotics, bactericidal drugs, bacteriostatic drugs, cleansing agents, absorbents, anti-infective agents, anti-inflammatory agents, astringents (drying agents that precipitate protein and shrink and contract the skin), pain killers, muscle relaxants, emollients (skin softeners), moisturizers, keratolytics (agents that soften, loosen, and facilitate exfoliation of the squamous cells of the epidermis), retinoids, salts thereof, and mixtures thereof.

### Routes of Administration

The pharmaceutical carriers herein are determined by the administration route. The present compounds and/or compositions may be administered parenterally by injection, orally and topically.

When administered topically, especially when the conditions addressed for treatment involve areas or organs readily accessible by topical application, including disorders of the eye, the skin, or the lower intestinal tract, suitable topical formulations are readily prepared for each of these areas.

For topical application to the skin, the compounds can be formulated in a suitable ointment containing the compound suspended or dissolved in, for example, a mixture with one or more of the following: mineral oil, liquid petrolatum, white petrolatum, propylene glycol, polyoxyethylene polyoxypropylene compound, emulsifying wax, and water. Alternatively, the compounds can be formulated in a suitable lotion or cream containing the active compound suspended or dissolved in, for example, a mixture of one or more of the following: mineral oil, sorbitan monostearate, polysorbate 60, cetyl esters wax, cetearyl alcohol, 2-octyldodecanol, benzyl alcohol, and water.

Topical application for the lower intestinal tract can be effected in a rectal suppository formulation (see above) or in a suitable enema formulation.

The term “parenteral” as used herein includes subcutaneous, intravenous, intramuscular, intraperitoneal, intrathecal, intraventricular, intrasternal, and intracranial injection or infusion techniques.

The present compounds and/or compositions may be administered in the form of sterile injectable preparations, for example, as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents, for example, as solutions in 1,3-butanediol. Among the acceptable vehicles and solvents that may be employed are water, Ringer's solution, and isotonic sodium chloride solution. In addition, sterile, fixed oils are conventionally employed as solvents or suspending mediums. For this purpose, any bland fixed oil may be employed including synthetic mono- or di-glycerides. Fatty acids such as oleic acid and its glyceride derivatives, including olive oil and castor oil, especially in their polyoxyethylated versions, are useful in the preparation of injectables. These oil solutions or suspensions may also contain long-chain alcohol diluents or dispersants.

For oral administration, the compounds described herein may be provided in any suitable dosage form known in the art. For example, the compositions may be incorporated into tablets, powders, granules, beads, chewable lozenges, capsules, gel caps, liquids, aqueous suspensions or solutions, or similar dosage forms, using conventional equipment and techniques known in the art. Tablet dosage forms are preferred. Tablets may contain carriers such as lactose and corn starch, and/or lubricating agents such as magnesium stearate. Capsules may contain diluents including lactose and dried corn starch. Aqueous suspensions may contain emulsifying and suspending agents combined with the active ingredient.

When preparing dosage forms incorporating the present compositions, the compounds may also be blended with conventional excipients such as binders, including gelatin, pregelatinized starch, and the like; lubricants, such as hydrogenated vegetable oil, stearic acid, and the like; diluents, such as lactose, mannose, and sucrose; disintegrants, such as carboxymethylcellulose and sodium starch glycolate; suspending agents, such as povidone, polyvinyl alcohol, and the like; absorbents, such as silicon dioxide; preservatives, such as methylparaben, propylparaben, and sodium benzoate; surfactants, such as sodium lauryl sulfate, polysorbate 80, and the like; colorants such as F.D.& C. dyes and lakes; flavorants; and sweeteners.

The present compounds may alternatively be administered by inhalation spray, rectally, nasally, buccally, vaginally, or via an implanted reservoir in dosage formulations containing conventional non-toxic pharmaceutically-acceptable carriers, adjuvants, and vehicles.

When administered by inhalation spray, these compositions may use metered dose inhalers and other pump or squeeze type sprays known to a person of ordinary skill in the art.

When administered rectally in the form of suppositories, these compositions can be prepared by mixing the drug with a suitable non-irritating excipient which is solid at room temperature, but liquid at rectal temperature and, therefore, will melt in the rectum to release the drug. Such materials include but are not limited to cocoa butter, beeswax, and polyethylene glycols.

The instant compositions and methods also may utilize controlled release technology. Thus, for example, the present compounds may be incorporated into a hydrophobic polymer matrix for controlled release over a period of days. Such controlled release films are well known to the art. Particularly preferred are transdermal delivery systems, such as transdermal patches and the like. Other examples of polymers commonly employed for this purpose that may be used herein include nondegradable ethylene-vinyl acetate copolymer and degradable lactic acid-glycolic acid copolymers which may be used externally or internally. Certain hydrogels such as poly(hydroxyethylmethacrylate) or poly(vinylalcohol) also may be useful, but for shorter release cycles then the other polymer releases systems, such as those mentioned above.

Other routes of administration known in the pharmaceutical art are also herein.

### EXAMPLE 46

### DA-5018 cream 0.3% w/w

A cream is prepared using conventional methods and formulated as follows:

| | |
|---|---|
| DA-5018 | 0.30 |
| Cetostearyl alcohol | 8.00 |
| Cetomacrogol 1000, BP | 3.00 |
| Polysorbate 80, EP | 2.00 |
| Isopropyl Myristate, EP | 15.00 |
| Carbomer, EP | 0.50 |
| Potassium dihydrogen phosphate | 0.41 |
| Sodium Hydroxide, EP | 0.28 |
| Glycerol, EP | 4.00 |
| Benzyl Alcohol, EP | 1.50 |
| Purified Water q.s. ca | 65.01 |
| | 100.00x |

### EXAMPLE 47

### DA-5018 cream 0.3% w/w

A cream is prepared using conventional methods and formulated as follows:

| | |
|---|---|
| DA-5018 | 0.300 |
| Cetostearyl alcohol | 8.00x |
| Propylene Glycol, EP | 6.00x |
| Cyclomethicone, USP/NF | 0.100 |
| Isopropyl Myristate, EP | 15.0xx |
| Diethyl glycol monoethyl ether, EP | 3.00x |
| Cetomacrogol 1000, BP | 3.00x |
| Polysorbate 80, EP | 2.00x |
| Carbomer, EP | 0.500 |
| Potassium phosphate, | 0.410 |
| monobasic anhydrous | |
| Sodium Hydroxide, EP | 0.280 |
| Methylparaben, EP | 0.180 |
| Propylparaben, EP | 0.020 |
| Purified Water q.s. ca | 61.21x |
| | 100.00x |

### EXAMPLE 48

### DA-5018 cream 0.3% w/w

A cream is prepared using conventional methods and formulated as follows:

| | |
|---|---|
| DA-5018 | 0.300 |
| Benzyl alcohol | 8.00x |
| Propylene Glycol, EP | 6.00x |
| Cyclomethicone, USP/NF | 0.100 |
| Isopropyl Myristate, EP | 15.0xx |
| Diethyl glycol monoethyl ether, EP | 3.00x |
| Cetomacrogol 1000, BP | 3.00x |
| Polysorbate 80, EP | 2.00x |
| Carbomer, EP | 0.500 |
| Potassium phosphate, | 0.410 |
| monobasic anhydrous | |
| Sodium Hydroxide, EP | 0.280 |
| Methylparaben, EP | 0.180 |
| Propylparaben, EP | 0.020 |
| Purified Water q.s ca | 61.21x |
| | 100.00x |

### EXAMPLE 49

### Tablet

Tablets are prepared by using conventional methods, e.g., mixing and direct compression, and formulated as follows:

| Ingredients | mg per tablet |
|---|---|
| DA-5018 | 10 |
| Compressible sugar (Di-pac) | 400 |
| Sodium starch glycolate | 35 |
| Silica Gel (Syloid 244) | 15 |

One tablet is administered orally to a patient (male; age: 42; weight: 67 kg) in need of analgesia two times daily to successfully provide the effect of general analgesia.

### EXAMPLE 50

### Capsule

Capsules for oral administration are prepared by combining the following ingredients:

| Ingredients | Amount | |
|---|---|---|
| DA-5018 | 20 | mg |
| Sesame oil | 100 | mL |

DA-5018 is dissolved in sesame oil with the aid of sonication and was packaged in soft gelatin capsules using the common methods known in the art. Two of the resulting capsules, each containing 27 mg of the composition, are administered to a 63 Kg male (age: 35) in need of treatment, producing the effects of analgesia and reducing inflammation.

### EXAMPLE 51

### Syrup

Syrup for oral administration is prepared by combining the following ingredients:

| Ingedients | Amount | |
|---|---|---|
| DA-5018 | 250 | g |
| Benzoic Acid Solution | 20 | mL |
| Compound Tartrative Solution | 10 | mL |
| Water for preparations | 20 | mL |
| Lemon syrup | 200 | mL |
| Syrup | to 1000 | mL |

The above ingredients are mixed to produce a syrup which is packaged under a sterile condition in 6 oz. bottles. One teaspoon of this formulation is administrated to a 70 kg male adult (age: 27), reducing inflammation and producing analgesia.

### EXAMPLE 52

### Injectable

Injectable compositions are prepared as follows:

| | |
|---|---|
| Ingredients | Amount |

Composition 1:

| | |
|---|---|
| DA-5018 | 0.01% |
| Aqueous Acetic Acid (1.30%) | 95.45% |
| Dextrose | 4.54% |

Composition 2:

| | |
|---|---|
| DA-5018 | 0.05% |
| Aqueous Sodium Acetate (1.18%) | 85.95% |
| Aqueous Acetic Acid (2.0%) | 10.00% |
| Benzyl alcohol | 4.04% |

The injection of 0.5 mL of Composition 2 prior to oral surgery for a third molar extraction of a female adult (weight: 52 kg; age: 29) successfully provided local anesthesia during the surgery.

### EXAMPLE 53

### Topical

A composition for topical administration is prepared by combining the following ingredients:

| Ingredients | Amount | |
|---|---|---|
| DA-5018 | 4 | g |
| Glycerol | 12 | mL |
| Purified water | 200 | mL |

DA-5018 is dissolved in a solution containing the other ingredients. Application of 0.4 mL of the resulting liquid to a 80 cm² portion of the forearm of a 60 kg male adult produced local analgesia which lasted for two days. Little or no skin irritation would be expected to be observed.

### Tests of Activity

Physiological activities of the compounds which can be prepared by the processes herein would be expected to be capable of measurement by employing methods well known in the art.

### EXAMPLE 54

### Writhing Test

1) Animals for Testing

The KTC-ICR mice derived from Charles River Breeding Laboratory in the United States and provided by Experimental Animal Laboratory of Korea Research Institute of Chemical Technology should preferably be used as test animals. The mice subjected to the testing of the end products can have a body weight of 10 to 25 g. They should be tested after having been adjusted to the testing environment for a week. Food and water should be given freely; and illumination maintained on a 12-hour cycle.

2) Testing Method

Experiments should be performed in two ways: that is, the acetic acid induced writhing test and the phenyl-1,4-benzoquinone (PBQ) induced writhing test.

Solutions for the acetic acid induced writhing test can be prepared by dissolving one of the end products in a saline solution containing 1% by weight of Tween 80 to have a concentration of 5 mg/mL and diluting it serially with the saline solution. The test solutions can be administered orally in a dose of 0.3 mL per 30 g of body weight, using the 5 ICR mice for each test. 60 minutes later, 0.9% acetic acid solution can be administered intraperitoneally in a dose of 0.1 mL per 30 g of body weight. 3 minutes thereafter, the number of writhings generated during a period of 10 minutes due to the administration of acetic acid should be measured. For comparison purposes, initially, saline solution alone can be administered orally to the control group. 60 minutes thereafter, 0.9% acetic acid solution can be administered intraperitoneally to the control group.

Alternatively, solutions for the PBQ induced writhing test can be prepared by dissolving one of the products synthesized in a mixture of Tween 80, alcohol and distilled water (1:5:94); and administered orally to the 5 ICR mice for each test in a dose of 0.3 mL per 30 g of body weight. 60 minutes later, 0.2% PBQ solution can be administered intraperitoneally in a dose of 0.1 mL per 30 g of body weight of the test animals. 5 minutes thereafter, at the temperature of 40° C., the number of writhings occurring during a period of 5 minutes due to the PBQ solution administered should be measured. For comparison, only the mixture of Tween 80, alcohol, and distilled water should be administered orally to the control group; and, after 60 minutes, the PBQ solution should be administered intraperitoneally to the control group in the same manner as mentioned above.

3) Measurement of Analgesic Effect

The number of writhings suffered by the test group can be compared with that of the control group; and the analgesic effect can be measured in terms of the percentage of inhibition of writhing (I.W.):
I.W. (%)=(A−B)/A×100

wherein

A is the number of writhings suffered by the control group; and

B is the number of writhings suffered by the test group.

The amount of a test compound shown to be required in reducing the frequency of writhings to the 50% level of that generated by the control group, i.e., B=0.5 A or I.W.=50%, is designated as ED₅₀; therefore, a lower value of ED₅₀ would represent a higher analgesic effect of the tested compound. It would be contemplated that capsaicinoid compounds produced herein would have the same effect, e.g. analgesia, as the identical compound made according to a prior but less desirable production method.

### EXAMPLE 55

### Behavior Analysis

In order to monitor a harmful side-effect or toxicity of the compounds, various behavioral changes in the test animals can be observed. After the test and the control solutions are administered to the animals, such symptoms as sedation, ptosis, dyspnoea, vasolidation, convulsion, salivation, and urination can be observed and the level of such changes can be represented by a numbering system; that is, the normal value of the last three behaviors (i.e., urination, convulsion, and salivation) is 0; and that of the others (i.e., sedation, ptosis, dyspnoea, and vasolidation) is 4. The higher the number, the greater the side effects.

### EXAMPLE 56

### Randal-Selitto Test

Randall-Selitto Test was carried out by following the method described in Arch. Int. Pharmacodyn., Vol. 11 (1957) p. 409, the contents of which are hereby incorporated by reference in their entirety.

Male albino rats (120-170 g) of the Charles River Sprague-Dawley strain are used. Inflammation is produced by the injection of 0.1 mL of a 20% suspension of Brewer's yeast into the plantar surface of the rat's hind foot. Thresholds are determined using a modified apparatus described in Winter and Flataker (J. Pharm. Exp. Ther., Vol. 148 (1965) p. 373), the contents of which are hereby incorporated by reference in their entirety.

The pain threshold is measured as the pressure in mm Hg required to induce the desired response (a sharp audible squeak and/or struggle) when the pressure is applied to the foot. Air pressure from an air line is admitted through a needle valve to a 20 mL glass syringe and to a pressure gauge connected by a T-tube. The syringe is mounted with the plunger downward to which is connected a short bullet-shaped Teflon peg. The pressure is applied to the foot of the rat at the rate of 10 mmHg per second. Drug is given 2 hours after the yeast injection. Two hours after the drug administration, threshold response is determined. The results are compared with the results obtained from the yeast-treated, and saline control group.

The analgesic activity was determined in terms of the percentage of inhibition of response:
Inhibition (%)=(TTG−TCG)/TCG×100,
where TTG is the Threshold of Treated Group, and TCG is the Threshold of the Control Group.

DA-5018, administered two hours after yeast injection and one hour before the test at a dose of 5 mg/kg perorally, caused an inhibition of yeast induced hyperalgesia.

| | | | Number | |
|---|---|---|---|---|
| Compound | Dose (mg/Kg) | | of rats | Inhibition (%) |
| Aspirin¹ | 100 | (s. c.) | 10 | 80.1 |
| Ketoprofen² | 10 | (p. o.) | 8 | 62.1 |
| Morphine³ | 3 | (s. c.) | 8 | 391.7 |
| DA-5018⁴ | 5 | (p. o.) | 5 | 247.4 |
| DA-5018-HCl⁴ | 1.5 | (s. c.) | 8 | 248.3 |

¹Bayer, U.S. Pat. No. 3,235,583
²RhonePoulanc, U.S. Pat. No. 3,641,127
³U.S. Pat. No. 2,740,787
⁴U.S. Pat. No. 5,242,944

### EXAMPLE 57

### Tail Flick Test

The tail flick assay of D'Amour and Smith (J. Pharm. Exp. Ther., Vol. 72 (1941) p. 74), the contents of which are hereby incorporated by reference in their entirety, was modified for use with mice. Radiant heat was applied using a beam of high-intensity light focused on a tail spot. The response time, defined as the interval between the onset of the stimulus and the tail flick, was measured electronically (to the nearest 0.1 second). The beam intensity was set at a level giving a mean control reaction time of 3.8±0.4 seconds. Animals that did not flick their tails within 15 seconds were removed and assigned a 15-second response latency.

The inhibition rates (analgesic effects) of the present compounds can be compared to standard compounds. The percentage of inhibition was determined by the following equation:
Inhibition (%)=(RTG−RCG)/RCG×100,
where RTG is the Reaction Time of Treated Group, and RCG is the Reaction Time of the Control Group.

Morphine used as reference was active in this test; but nonsteroidal anti-inflammatory drugs were ineffective. The results reported in U.S. Pat. No. 5,242,944, the entire contents of which are hereby incorporated by reference, show that DA-5018 is more effective than morphine and suggest that DA-5018 behaves as a central analgesic.

| Compound | Dose (mg/Kg) | Inhibition (%) |
|---|---|---|
| Aspirin (p. o.) | 100 | 0 |
| Piroxicam (p. o.)¹ | 100 | 0 |
| Capsaicin | 25 | 90 |
| Morphine-HCl (s. c.) | 2.0 | 57 |
| | 5.0 | 100 |
| NE-21610 (p. o.)² | 200 | 50 |
| Example 1 (s. c.) | 0.5 | 50 |
| | 1.0 | 70 |
| DA-5018 (p. o.) | 1.25 | 10 |
| | 2.5 | 60 |
| | 5.0 | 80 |
| | 7.5 | 90 |
| DA-5018-l-tartarate | 1.25 | 50 |
| (p. o.) | 2.5 | 90 |

¹Pfizer, U.S. Pat. No. 3,591,584
²P & G, U.S. Pat. No. 5,045,565

### EXAMPLE 58

### Hot-Plate Test

Mice were placed on an aluminum plate maintained at 55×0.5° C. by a thermo-regulator (Harvard). A glass cylinder, 15 cm in height and 15 cm in diameter, served to confine the mice to the heated plate. Blowing of the fore paws was used as the end-point for determination of response latency (measured to the nearest 0.1 second). Animals which failed to react within 30 seconds were removed and assigned a 30-second response latency.

The inhibition rates of DA-5018 (p. o.) and morphine (s. c.) in the hot plate test were determined by the same equation as used in Tail-flick Test, which are shown above in Example 57. The results reported in U.S. Pat. No. 5,242,944 show that DA-5018 (p. o.) is as effective as morphine and also suggest that DA-5018 behaves as a central analgesic.

| Compound | Dose (mg/Kg) | Inhibition (%) |
|---|---|---|
| Morphine-HCl (s. c.) | 2.0 | 27.5 |
| | 5.0 | 49.0 |
| | 10.0 | 98.0 |
| DA-5018 (p. o.) | 2.5 | 28.6 |
| | 5.0 | 48.9 |
| | 10.0 | 97.0 |

### EXAMPLE 59

### Tail-Pinch Test in Rats with Hyperalgesia Induced by Freund's Adjuvant

Rats (Sprague-Dawley) weighing 120 g to 170 g were used. Desiccated Mycobacterium butyricum (Difco Laboratories, Detroit, Mich.) was ground in a mortar, suspended in liquid paraffin, sterilized in an autoclave, and injected (0.5 mg in 0.1 mL, s.c.) in the distal region of the tail through a 1-inch 21-gauge needle.

Animals so treated exhibited hypersensitivity to the pressure placed on the tail within a few hours of the injection and were for analgesic testing 18 to 24 hours after injection. The hypersensitivity of the tail was examined as follows: the animal was held comfortably in one hand and gentle pressure was applied with the fingers of the opposite hand to the injected area. This gentle squeeze or “tail pinch” elicited a “squeak” from the animal. Five such stimuli were given at 4-second intervals. If the animal emitted no more than one squeak in five trials, it was recorded as having analgesia and given a rating of 1. If there was more than one squeak, the rating was given the value of 0.

The analgesic activity was determined by the following equation:
Analgesic activity=(Total rating/tested animal number)×100

DA-5018, administered two hours before tail-pinch testing perorally, caused a dose related inhibition of adjuvant induced hyperalgesia, as reported in U.S. Pat. No. 5,242,944 and as shown below.

| | Dose | Number | Analgesic |
|---|---|---|---|
| Compound | (mg/Kg) | of rats | activity (%) |
| Naproxen¹ | 5 | 7 | 28.6 |
| DA-5018² | 5 | 7 | 45.9 |
| | 10 | 6 | 66.7 |

¹Syntex, U.S. Pat. No. 3,637,767
²U.S. Pat. No. 5,242,944

### EXAMPLE 60

### Anti-Inflammatory Test

Rats (Sprague-Dawley, female) weighing 100 to 120 g were used. Twenty minutes after the test drug was administered (s. c.), carrageenan was injected (0.1 mL of 1% solution, s. c.) in the plantar surface of the right hand paw. The volume of the edema was measured with a volumeter (Rehma Volumeter 2060) 3 hours later.

The percentage of inhibition was determined by the following equation:
Inhibition (%)=(VTF−VCF)/VCF×100
where VTF is the Volume of the (carrageenan) Treated Foot, and VCF is the Volume of the Control Foot.

The amount of a test compound which is required in obtaining 50% inhibition is designated as ED₅₀.

The inhibitory effects of DA-5018 were significantly superior to that of Aspirin or Naproxen, as reported in U.S. Pat. No. 5,242,944.

### EXAMPLE 61

### Local Anti-Inflammatory Test

Rats (Sprague-Dawley, female) weighing 100 to 120 g were used. Twenty minutes after the test drug was administered transdermally (another application 7 hours later for double dose experiment), carrageenan was injected (0.1 mL of 1% solution, s. c.) into the plantar surface of the right hind paw. The volume of the edema was measured with a volumeter either 1 hour later for single dose experiment or 24 hours later for double dose experiment; and the percentage of inhibition was be measured by the same equation as used in Anti-inflammatory Test.

DA-5018, administered transdermally, would be expected to cause a dose related inhibition of carrageenan induced hyperalgesia, as reported in U.S. Pat. No. 5,242,944.

### EXAMPLE 62

### Test of Toxicity

In addition, the acute toxicity test for the compounds may be carried out at LD₅₀ by per os administering the test compound in varied amounts in a stepwise manner into 5 ICR male mice and 5 ICR female mice (5 weeks old), which are observed for 14 days. The LD₅₀ values of the compounds would then be calculated in mg/Kg for the male mice and female mice).

### EXAMPLE 63

### Activity Screening—VR1 Agonism

Since the vanilloid receptor VR1 is a cation permeable ion channel present on nociceptors and has been cloned from rat and human, it can be used as an additional screening method to determine the activity of vanilloid receptor ligands prepared according to the present subject matter.

Using standard techniques, VR1 transfected cells, such as CHO cells, are used to determine the agonist activity of VR1 ligands as compared to a standard VR1 ligand such as capsaicin. Binding affinities, represented as Ki, can be assessed by the specific ability of ligands/compounds to compete with [3H]RTX in the VR1-cell system. Resiniferatoxin (RTX) is also, like capsaicin, a known VR1 ligand. Tritiated RTX studies and competitive Ki binding activities can be carried out according to Szallasi et al. and others, as is known in the art.

The present subject matter being thus described, it will be obvious that the same may be modified or varied in many ways. Such modifications and variations are not to be regarded as a departure from the spirit and scope of the present subject matter, and all such modifications and variations are intended to be included within the scope of the following claims.

We claim:

1. A polymorph or a hydrate of DA-5018.

2. The polymorph or hydrate of claim 1, which comprises chemical, physical, mechanical, electrical, thermodynamic, and biological characteristics selected from the range consisting of storage stability, compressibility, density, dissolution rate, solubility, melting point, chemical stability, physical stability, powder flowability, compaction, and particle morphology.

3. A substantially pure polymorph of Form II of DA-5018.

4. The substantially pure polymorph of Form II of DA-5018 of claim 3, which is substantially devoid of polymorphic or hydrate Forms I, III, IV, or V as determined on a % weight basis.

5. The substantially pure polymorph of Form II of DA-5018 of claim 4, which has less than about 5% by weight of polymorphic or hydrate Forms I, III, IV, or V as determined on a % weight basis.

6. The substantially pure polymorph of claim 3, wherein the polymorph Form II of DA-5018 has at least 95% purity as defined by X-ray powder diffraction.

7. The substantially pure polymorph of claim 3, wherein the polymorph Form II of DA-5018 has characteristic X-ray powder diffraction (XRPD) 2-theta positions at about 5.0, 9.4, 12.9, 14.9, 16.3, 17.5, 22.8, and 25.0.

8. The polymorph or hydrate of DA-5018 of claim 1, wherein said polymorph or hydrate is selected from the group consisting of a substantially pure polymorph of Form I of DA-5018, a substantially pure dihydrate of Form III of DA-5018, a substantially pure polymorph of Form IV of DA-5018, and a substantially pure polymorph of Form V of DA-5018.

9. The substantially pure polymorph of Form I of DA-5018 of claim 8, which is substantially devoid of polymorphic or hydrate Forms II, III, IV, or V as determined on a % weight basis.

10. The substantially pure polymorph of claim 8, wherein the polymorph Form I of DA-5018 has at least 95% purity as defined by X-ray powder diffraction.

11. The substantially pure polymorph of claim 8, wherein the polymorph Form I of DA-5018 has characteristic X-ray powder diffraction (XRPD) 2-theta positions at about 7.8, 11.0, 13.7, 14.9, 15.4, 16.6, 19.0, 20.8, 22.2, and 25.0.

12. The substantially pure dihydrate of Form III of DA-5018 of claim 8, which is substantially devoid of polymorphic Forms I, II, IV, or V as determined on a % weight basis.

13. The substantially pure dihydrate of claim 8, wherein the dihydrate Form III of DA-5018 has at least 95% purity as defined by X-ray powder diffraction.

14. The substantially pure dihydrate of claim 8, wherein the dihydrate Form III of DA-5018 has characteristic X-ray powder diffraction (XRPD) 2-theta positions at about 8.2, 14.2, 16.2, 20.2, 21.9, 22.9, 23.5, and 25.1.

15. The substantially pure polymorph of Form IV of DA-5018 of claim 8, which is substantially devoid of polymorphic or hydrate Forms I, II, III, or V as determined on a % weight basis.

16. The substantially pure polymorph of claim 8, wherein the polymorph Form IV of DA-5018 has at least 95% purity as defined by X-ray powder diffraction.

17. The substantially pure polymorph of claim 8, wherein the polymorph Form IV of DA-5018 has characteristic X-ray powder diffraction (XRPD) 2-theta positions at about 7.2, 8.5, 9.3, 13.5, 17.3, 21.1, 22.7, 24.6, 25.3, and 26.2.

18. The substantially pure polymorph of Form V of DA-5018 of claim 8, which is substantially devoid of polymorphic or hydrate Forms I, II, III, or IV as determined on a % weight basis.

19. The substantially pure polymorph of claim 8, wherein the polymorph Form V of DA-5018 has at least 95% purity as defined by X-ray powder diffraction.

20. The substantially pure polymorph of claim 8, wherein the polymorph Form V of DA-5018 has characteristic X-ray powder diffraction (XRPD) 2-theta positions at about 7.8 and 24.9.

21. A pharmaceutical composition, which comprises:
a polymorph or hydrate of claim 1; and
a pharmaceutically acceptable carrier.

22. A method of treating a skin disorder which comprises administering to a patient in need thereof an effective amount of the pharmaceutical composition of claim 21.

23. The method of claim 22, wherein the skin disorder is selected from the group consisting of neuralgias, inflammatory disorders, pruritis, hyperproliferative skin diseases, diseases involving skin metabolism, infections, excretions, improvement in the skin appearance and health, and combinations thereof.

24. The method of claim 23, wherein the skin disorder is selected from the group consisting of post herpetic neuralgia, pruritis, pruritis associated with atopic dermatitis, acne, rosacea, atopic dermatitis, psoriasis, eczema, seborrheic dermatitis, pyodermas, neurodermatitis, intertrigo, pruritis, tinea infections, verrucum, warts, viral infections, herpes simplex infections, impetigo, and combinations thereof.

25. The method of claim 22, wherein said administering of said pharmaceutical composition results in an improvement of the patient's condition, reduction of symptoms, an improvement in the patient's appearance, or combinations thereof.

26. The method of claim 22, wherein said skin disorder exhibits an observable symptom selected from the group consisting of inflammation, erythema, swelling, pain, pruritis, cell hyperproliferation, telangiectasia, pyoderma, hyperpigmentation, bacterial fungal or viral infection, skin lesions, redness, pustules, cysts, nodules, papules, hypertrophy of the sebaceous glands, and combinations thereof.

27. A process for producing polymorph II of crystalline DA-5018, which comprises:
i) dissolving crude DA-5018 in an appropriate solvent to obtain a solution;
ii) filtering the solution of step i) to obtain a filtrate;
iii) treating the filtrate with activated carbon to obtain an activated carbon mixture;
iv) filtering the activated carbon mixture and obtaining a residue therefrom;
v) suspending the residue in an appropriate solvent or mixture of solvents to obtain a suspension;
vi) heating the suspension until a heated solution is obtained;
vii) allowing the heated solution to cool over time and a product to crystallize to form a second suspension;
viii) filtering the second suspension to obtain a filter-cake;
ix) washing the filter-cake; and
x) drying the filter-cake to obtain purified DA-5018 polymorph Form II.

28. The process of claim 27, wherein the solvent or mixture of solvents is selected from the group consisting of isopropyl acetate, ethyl acetate, methanol, ethanol, acetonitrile, water, and mixtures thereof.

29. A process for reducing a nitrile to obtain an amine compound, which comprises:
catalytically hydrogenating a nitrile compound in a dipolar aprotic organic solvent in the presence of a palladium/carbon catalyst and a strong anhydrous protic acid to obtain an amine compound.

30. The process of claim 29, wherein the process is carried out at a reaction temperature of from about −10° C. to about 25° C.

31. The process of claim 30, wherein the reaction temperature is from about 0° C. to about 10° C.

32. The process of claim 29, wherein the palladium/carbon catalyst has a concentration of from about 0.1% to about 20% palladium on carbon.

33. The process of claim 32, wherein the palladium/carbon catalyst has a concentration of about 5% palladium on carbon.

34. The process of claim 29, wherein the palladium/carbon catalyst is in suspension or a dispersion and has a catalyst loading of about 0.1% to about 50% by weight.

35. The process of claim 34, wherein the palladium/carbon catalyst has a catalyst loading of about 5% to about 20% by weight.

36. The process of claim 29, wherein the dipolar aprotic organic solvent is selected from the group consisting of DMA, HMPA, DMPU, THF, NMP, DMF, DMSO, sulfolane, and mixtures thereof.

37. The process of claim 36, wherein the dipolar aprotic organic solvent is from about 0.1% to about 30% NMP in THF.

38. The process of claim 37, wherein the dipolar aprotic organic solvent is about 10% NMP in THF.

39. The process of claim 29, wherein the strong anhydrous protic acid is selected from the group consisting of sulfuric acid, alkylsulfonic acids, arylsulfonic acids, phosphoric acids, alkylphosphoric acids, arylphosphoric acids, perfluoroalkylcarboxylic acids, pentafluoroalkylcarboxylic acids, hypophosphorous acids, and mixtures thereof.

40. The process of claim 39, wherein said strong anhydrous protic acid is selected from the group consisting of trifluoroacetic acid, methanesulfonic acid, sulfuric acid, and mixtures thereof.

41. The process of claim 39, where the strong anhydrous protic acid has a concentration of from about 0.1 molar eq. to about 10 molar eq.

42. The process of claim 40, wherein the strong anhydrous protic acid is methanesulfonic acid or sulfuric acid and the strong anhydrous protic acid has a concentration of about 1.6 molar eq.

43. The process of claim 29, wherein the reaction is under hydrogen pressure of from about 5 psig to about 300 psig.

44. The process of claim 43, wherein the hydrogen pressure is from about 10 psig to about 100 psig.

45. The process of claim 44, wherein the hydrogen pressure is about 50 psig.

46. The process of claim 44, wherein the hydrogen pressure is about 16 psig.

47. The process of claim 29, wherein the process affords from about 50% to about 99% pure amine product.

48. The process of claim 47, wherein the process affords from about 85% to about 99% pure amine product.

49. The process of claim 47, wherein the process affords an amine product with over about 85% purity.

50. An amine product prepared by the process of claim 29, wherein the amine product has a purity of about 50% to about 99% pure amine product and is selected from the group consisting of a pesticide, herbicide, propellant, polymer, reagent, fungicide, fumigant, plant growth regulator, insecticide, PEG-ylated compound, intermediates thereof, and mixtures thereof.

51. The product by process of claim 50, wherein the process has a yield of over about 50%.

52. The product by process of claim 50, wherein the process has a yield of over about 80%.

53. An amine product prepared by the process of claim 29, wherein the amine product has a purity of about 85% to about 99% pure amine product and is selected from the group consisting of a pharmaceutical, preservative, drug modifier, intermediates thereof, and mixtures thereof.

54. The product by process of claim 53, wherein the process has a yield of over about 50%.

55. The product by process of claim 53, wherein the process has a yield of over about 80%.

56. A process for reducing a nitrile to obtain an amine compound, which comprises:
catalytically hydrogenating a nitrile compound in a dipolar aprotic organic solvent in the presence of a palladium/carbon catalyst and a strong anhydrous protic acid; and
obtaining an amine compound;
wherein the dipolar aprotic organic solvent is selected from the group consisting of THF, NMP, DMF, DMSO, sulfolane, and mixtures thereof,
wherein the strong anhydrous protic acid has a concentration of from about 0.1 molar eq. to about 10 molar eq. and is selected from the group consisting of trifluoroacetic acid, sulfuric acid, alkylsulfonic acid, arylsulfonic acid, phosphoric acid, alkylphosphoric acid, arylphosphoric acid, and mixtures thereof,
wherein the process is carried out at a reaction temperature of from about 0° C. to about 10° C.,
wherein the palladium/carbon catalyst has a concentration of from about 0.1% to about 20% palladium on carbon, and
wherein the process is carried out at a hydrogen pressure of from about 10 psig to about 100 psig.

57. The process of claim 56, wherein the dipolar aprotic organic solvent is from about 0.1% to about 30% NMP in THF.

58. A product by the process of claim 56, wherein the product has a purity of about 85% to about 99% pure amine product.

59. A product by the process of claim 56, wherein the process has a yield of over about 85%.

60. A process of preparing an amine compound, which comprises: 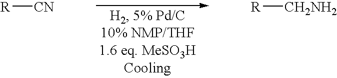
wherein NMP/THF is anhydrous, R—CN is a nitrile-containing compound subjected to reduction to provide the amine end product R—CH₂NH₂, and R is an organic compound.

61. A product by the process of claim 60, wherein the product has a purity of about 85% to about 99% pure amine product.

62. A product by the process of claim 60, wherein the process has a yield of over about 85%.

63. A process for preparation of an amine product, which comprises:
catalytically hydrogenating a nitrile compound of Formula Ia: 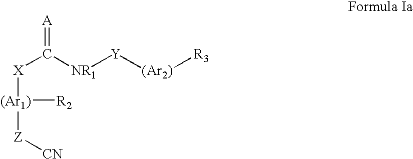
in a dipolar aprotic organic solvent in the presence of a palladium/carbon catalyst and a strong anhydrous protic acid; and
obtaining the amine product, wherein
X is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;
Y is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;
Z is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;
A is oxygen or a sulfur wherein the sulfur is optionally substituted with 2 or 4 hydrogen, oxy, alkyl, alkyloxy, or alkylamino radicals;
R₁ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;
Ar₁ is a heterocycle, aryl, or heteroaryl radical wherein Ar₁ is substituted in one to five places with R₂;
R₂ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;
Ar₂ is a heterocycle, aryl, or heteroaryl radical wherein Ar₂ is substituted in one to five places with R₃;
R₃ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;
R₄ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo;
R₅ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo; and
wherein said heterocycle is a radical of a monocyclic or bicyclic saturated heterocyclic ring system having 5-8 ring members per ring, wherein 1-3 ring members are oxygen, sulfur or nitrogen heteroatoms, which is optionally partially unsaturated or benzo-fused and optionally substituted by 1-2 oxo or thioxo radicals; said aryl is a phenyl or naphthyl radical; and said heteroaryl is a radical of a monocyclic or bicyclic aromatic heterocyclic ring system having 5-6 ring members per ring, wherein 1-3 ring members are oxygen, sulfur or nitrogen heteroatoms, which is optionally benzo-fused or saturated C₃-C₄-carbocyclic-fused.

64. The process of claim 63, wherein:
X is a C₁₋₁₀ alkyl or C₂₋₁₀ alkenylene radical;
Y is a C₁₋₂₀ alkyl or C₂₋₁₀ alkenylene radical;
Z is a C₁₋₂₀ alkyl, C₁₋₂₀ alkyloxy, C₂₋₂₀ alkenylene, or C₂₋₂₀ alkenoxy radical;
A is oxygen or sulfur;
R₁ is hydrogen, C₁₋₂₀ alkyl, or C₂₋₂₀ alkenylene;
Ar₁ is a C₃₋₂₀ carbocyclic ring or C₃₋₂₀ heterocyclic ring having one or more heteroatoms selected from O, N, or S, wherein Ar₁ is substituted in one to five places with R₂;
R₂ is hydrogen, C₁₋₂₀ alkyl, C₂₋₂₀ alkenylene, C₁₋₂₀ alkyloxy, C₂₋₂₀ alkenoxy, C₁₋₂₀ thioalkyl, or C₂₋₂₀ thioalkenylene;
Ar₂ is a C₃₋₂₀ carbocyclic ring or C₃₋₂₀ heterocyclic ring having one or more heteroatoms selected from O, N, or S, wherein Ar₂ is substituted in one to five places with R₃; and
R₃ is hydrogen, C₁₋₂₀ alkyl, C₂₋₂₀ alkenylene, C₁₋₂₀ alkyloxy, C₂₋₂₀ alkenoxy, C₁₋₂₀ thioalkyl, or C₂₋₂₀ thioalkenylene.

65. The process of claim 63, wherein the process is carried out at a reaction temperature of from about −10° C. to about 25° C.

66. The process of claim 65, wherein the reaction temperature is from about 0° C. to about 10° C.

67. The process of claim 63, wherein the palladium/carbon catalyst has a concentration of from about 0.1% to about 20% palladium on carbon.

68. The process of claim 67, wherein the concentration of the palladium/carbon catalyst is about 5% palladium on carbon.

69. The process of claim 63, wherein the palladium/carbon catalyst has a catalyst loading of about 0.1% to about 50% by weight.

70. The process of claim 69, wherein the concentration of palladium/carbon catalyst has a catalyst loading of about 5% to about 20% by weight.

71. The process of claim 63, wherein the dipolar aprotic organic solvent is selected from the group consisting of THF, NMP, DMF, DMSO, sulfolane, and mixtures thereof.

72. The process of claim 71, wherein the dipolar aprotic organic solvent is from about 0.1% to about 30% NMP in THF.

73. The process of claim 72, wherein the dipolar aprotic organic solvent is about 10% NMP in THF.

74. The process of claim 63, wherein the strong anhydrous protic acid is selected from the group consisting of trifluoroacetic acid, sulfuric acid, alkylsulfonic acid, arylsulfonic acid, phosphoric acid, alkylphosphoric acid, arylphosphoric acid and mixtures thereof.

75. The process of claim 74, wherein the strong anhydrous protic acid is methanesulfonic acid or sulfuric acid.

76. The process of claim 74, where the strong anhydrous protic acid has a concentration of from about 0.1 molar eq. to about 10 molar eq.

77. The process of claim 75, wherein the strong anhydrous protic acid is methanesulfonic acid or sulfuric acid and has a concentration of about 1.6 molar eq.

78. The process of claim 63, wherein the process is carried out at a hydrogen pressure of from about 5 psig to about 300 psig.

79. The process of claim 78, wherein the hydrogen pressure is from about 10 psig to about 100 psig.

80. The process of claim 79, wherein the hydrogen pressure is about 50 psig.

81. The process of claim 79, wherein the hydrogen pressure is about 16 psig.

82. The process of claim 63, wherein the process affords from about 50% to about 99% pure amine product.

83. The process of claim 82, wherein the process affords from about 85% to about 99% pure amine product.

84. The process of claim 82, wherein the process affords an amine product with over about 85% purity.

85. The process of claim 63, wherein the nitrile compound has the following formula:

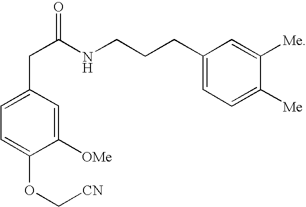

86. An amine product prepared by the process of claim 63, which has a purity of about 85% to about 99% pure.

87. The product by process of claim 86, wherein the process has a yield of over about 50%.

88. The product by process of claim 86, wherein the process has a yield of over about 80%.

89. The process of claim 63, wherein the amine product has the following formula:

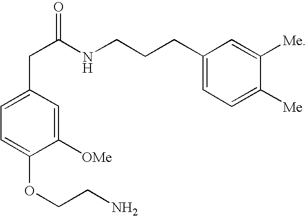

90. A process for preparation of DA-5018, which comprises:
catalytically hydrogenating a nitrile compound of the formula:

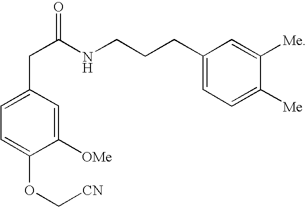

91. DA-5018 prepared by the process of claim 90, which is at least about 85% pure.

92. DA-5018 of claim 91, which is at least about 90% pure.

93. DA-5018 of claim 91, which is at least about 95% pure.

94. A process for preparing an amine compound, which comprises:
catalytically hydrogenating a nitrile compound of the formula: 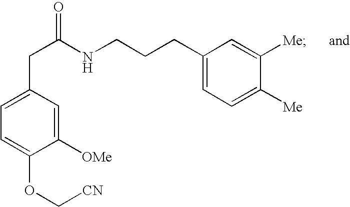
obtaining an amine product of the formula:

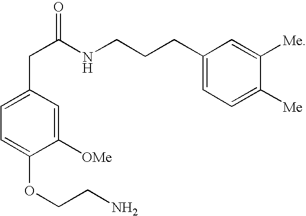

95. A compound which is useful in the manufacture of capsaicinoids, which comprises:

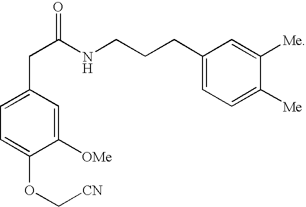

96. A process for preparation of an amine product, which comprises:
deprotecting a compound of Formula II: 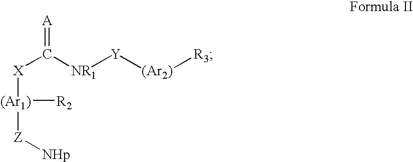
and obtaining the amine product, wherein
X is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;
Y is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;
Z is a bond, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;
A is oxygen or sulfur wherein the sulfur is optionally substituted with 2 or 4 hydrogen, oxy, alkyl, alkyloxy, or alkylamino radicals;
R₁ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;
Ar₁ is a heterocycle, aryl, or heteroaryl radical wherein Ar₁ is substituted in one to five places with R₂;
R₂ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;
Ar₂ is a heterocycle, aryl, or heteroaryl radical wherein Ar₂ is substituted in one to five places with R₃;
R₃ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo, wherein 1-3 carbons of the alkyl, alkenylene, or alkynylene are optionally replaced with O, NR₄, or SR₅;
R₄ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo;
R₅ is a hydrogen radical, or an alkyl, alkenylene, or alkynylene radical, each of which is straight or branched and is optionally substituted by 1-3 radicals of alkoxy, alkenoxy, hydroxy, amino, alkylamino, dialkylamino, alkanoylamino, alkoxycarbonylamino, alkylsulfonylamino, nitro, nitrile, azido, thio, alkylthio, alkylsulfinyl, sulfonyl, heterocycle, aryl, heteroaryl, or halo; and
p is a protecting group;
wherein said heterocycle is a radical of a monocyclic or bicyclic saturated heterocyclic ring system having 5-8 ring members per ring, wherein 1-3 ring members are oxygen, sulfur or nitrogen heteroatoms, which is optionally partially unsaturated or benzo-fused and optionally substituted by 1-2 oxo or thioxo radicals; said aryl is a phenyl or naphthyl radical; and said heteroaryl is a radical of a monocyclic or bicyclic aromatic heterocyclic ring system having 5-6 ring members per ring, wherein 1-3 ring members are oxygen, sulfur or nitrogen heteroatoms, which is optionally benzo-fused or saturated C₃-C₄-carbocyclic-fused.

97. The process of claim 96, wherein
X is a C₁₋₁₀ alkyl or C₂₋₁₀ alkenylene radical;
Y is a C₁₋₂₀ alkyl or C₂₋₁₀ alkenylene radical;
Z is a C₁₋₂₀ alkyl, C₁₋₂₀ alkyloxy, C₂₋₂₀ alkenylene, or C₂₋₂₀ alkenyleneoxy radical;
A is oxygen or sulfur;
R₁ is hydrogen, C₁₋₂₀ alkyl, or C₂₋₂₀ alkenylene;
Ar₁ is a C₃₋₂₀ carbocyclic ring or C₃₋₂₀ hetercyclic ring having one or more heteroatoms selected from O, N, or S, wherein Ar₁ is substituted in one to five places with R₂;
R₂ is hydrogen, C₁₋₂₀ alkyl, C₂₋₂₀ alkenylene, C₁₋₂₀ alkyloxy, C₂₋₂₀ alkenyleneoxy, C₁₋₂₀ thioalkyl, or C₂₋₂₀ thioalkenylene;
Ar₂ is a C₃₋₂₀ carbocyclic ring or C₃₋₂₀ hetercyclic ring having one or more heteroatoms selected from O, N, or S, wherein Ar₂ is substituted in one to five places with R₃;
R₃ is hydrogen, C₁₋₂₀ alkyl, C₂₋₂₀ alkenylene, C₁₋₂₀ alkyloxy, C₂₋₂₀ alkenyleneoxy, C₁₋₂₀ thioalkyl, or C₂₋₂₀ thioalkenylene; and
p is t-butyloxycarbonyl (t-Boc).

98. A process for preparation of DA-5018, which comprises:
1) deprotecting a compound of Formula III: 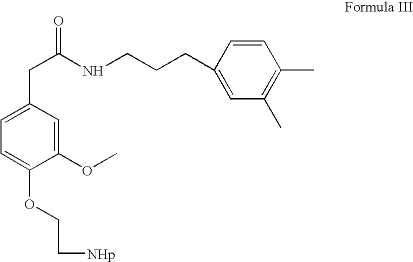
wherein p is a protecting group; and
2) obtaining DA-5018.

99. DA-5018 prepared by the process of claim 98, which is at least about 85% pure.

100. DA-5018 of claim 99, which is at least about 90% pure.

101. DA-5018 of claim 99, which is at least about 95% pure.

102. A compound of Formula IV which is useful in the manufacture of capsaicinoids, which comprises: 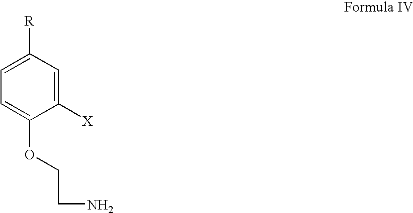
wherein
R is C₁₋₆ alkyl or C₂₋₆ alkenylene substituted with COOH or CONH₂, and
X is C₁₋₁₀ alkoxy, C₂₋₁₀ alkenoyl, or C₂₋₁₀ alkenoxy,
provided that R is not C₁—COOH when X is methoxy.

103. A compound which is useful in the manufacture of capsaicinoids, which comprises: 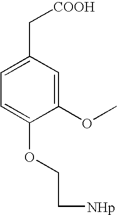
wherein p is a protecting group.

104. A compound which is useful in the manufacture of capsaicinoids, which comprises: 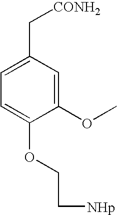
wherein p is a protecting group.

105. A compound which is useful in the manufacture of capsaicinoids, which comprises: 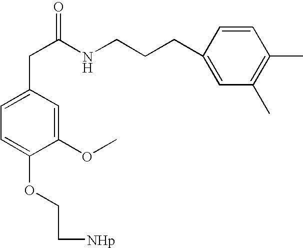
wherein p is a protecting group.

106. The compound of claim 109, wherein p is t-Butyloxycarbonyl (t-Boc).

107. A process for preparing an amine product, which comprises:
deprotecting Compound A: 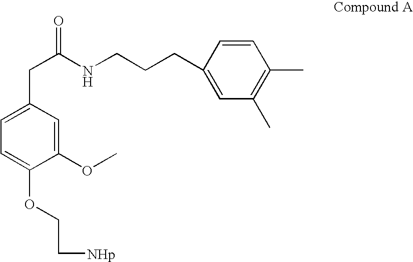
wherein p is a protecting group; and
obtaining the amine product, Compound B:

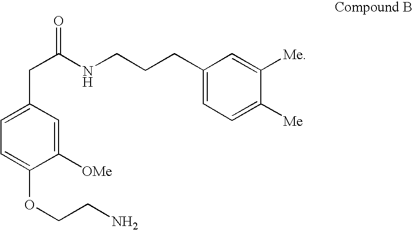

108. An amine product prepared according to the process of claim 107, which is at least about 85% pure.

109. The amine product of claim 108, which is at least about 90% pure.

110. The amine product of claim 108, which is at least about 95% pure.

111. A pharmaceutical composition, which comprises:
a product prepared by the process of claim 29; and
a pharmaceutically acceptable carrier.

112. A method of treating a skin disorder which comprises administering to a patient in need thereof an effective amount of the pharmaceutical composition of claim 111.

113. The method of claim 112, wherein the skin disorder is selected from the group consisting of neuralgias, inflammatory disorders, pruritis, hyperproliferative skin diseases, diseases involving skin metabolism, infections, excretions, improvement in the skin appearance and health, and combinations thereof.

114. The method of claim 113, wherein the skin disorder is selected from the group consisting of post herpetic neuralgia, pruritis, pruritis associated with atopic dermatitis, acne, rosacea, atopic dermatitis, psoriasis, eczema, seborrheic dermatitis, pyodermas, neurodermatitis, intertrigo, pruritis, tinea infections, verrucum, warts, viral infections, herpes simplex infections, impetigo, and combinations thereof.

115. The method of claim 112, wherein said administering of said pharmaceutical composition results in an improvement of the patient's condition, reduction of symptoms, an improvement in the patient's appearance, or combinations thereof.

116. The method of claim 112, wherein said skin disorder exhibits an observable symptom selected from the group consisting of inflammation, erythema, swelling, pain, pruritis, cell hyperproliferation, telangiectasia, pyoderma, hyperpigmentation, bacterial fungal or viral infection, skin lesions, redness, pustules, cysts, nodules, papules, hypertrophy of the sebaceous glands, and combinations thereof.
